(19) United States
(12) Patent Application Publication
(10) Pub. No.: US 2024/0246905 A1
(43) Pub. Date: Jul. 25, 2024

(54) VINYL ISOCYANIDE COMPOUNDS AS ANTIBACTERIAL AGENTS

(71) Applicant: University of Leicester, England (GB)

(72) Inventors: Steven D. Bull, Somerset (GB); Liam J. Stephens, Somerset (GB)

(73) Assignee: University of Leicester, England (GB)

(21) Appl. No.: 18/290,241

(22) PCT Filed: May 10, 2022
(86) PCT No.: PCT/GB2022/051181

§ 371 Date: Nov. 10, 2023

(30) Foreign Application Priority Data
May 13, 2021 (GB) 2106873.9

(51) Int. Cl. C07C 291/10; A61K 31/277; A61K 31/4409; A61K 31/505; A61P 31/04; C07D 213/57; C07D 239/26

(52) U.S. Cl. CPC C07C 291/10; A61K 31/277; A61K 31/4409; A61K 31/505; A61P 31/04; C07D 213/57; C07D 239/26; C07C 2601/16

(57) ABSTRACT

Compounds of formula (I) or formula (II) are disclosed:

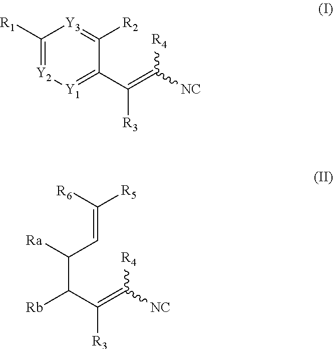

Wherein Y₁, Y₂ and Y₃ are independently selected from C—R₁ or N. Such compounds find use as antibiotics and antifungals.

### FIELD OF THE INVENTION

The present invention relates to compounds that find use in treating infectious disease, more particularly as antibiotics and antifungals, to methods of producing such compounds, and to reagents for use in such methods.

### BACKGROUND

The emergence of antimicrobial-resistant (AMR) bacteria represents a serious threat to human health. There is currently a lack of new antibiotics being developed, in particular those that can target gram positive bacterial biofilms.⁽²⁾ There is in addition an urgent need for new drugs to treat multidrug resistance in bacteria and fungi.

WO-A-2008/124836 discloses methods and compounds for controlling virulence in bacteria, methods of identifying further compounds for controlling virulence in bacteria, and methods, compounds, and compositions for treating subjects with bacterial infections to reduce virulence of bacteria in said subjects. EP-A-0 440 887 discloses processes for the preparation of Erbstatin and Erbstatin analogs. WO-A-2021/145729 discloses a pharmaceutical composition for the prevention or treatment of cancer, inflammatory disease or metabolic disease.

Microbial biofilms are the community of microbial cells immersed and protected within an extracellular polymeric matrix and are difficult to disrupt. Biofilms can form on biotic and abiotic surfaces ranging from heart valves, to implanted medical devices such as catheters and prosthesis. It is estimated that 80% of internal bacterial infections are associated with biofilms.⁽⁴⁾ Biofilm associated infections may be refractory to antibiotic concentrations of up to 1000-fold higher than planktonic minimum inhibition concentration (MIC).⁽⁵⁾ There is therefore a need for antibiotics that are useful in reducing, preventing and/or eradicating bacterial biofilms.

There is therefore a need to provide antibiotics and antifungals that address problems with the prior art and are able to prevent or treat bacterial and other infections and, in particular, are effective against biofilms.

It is an aim of the present invention to address this need.

### SUMMARY

In a first aspect, there is accordingly provided a compound of formula (I) or formula (II):

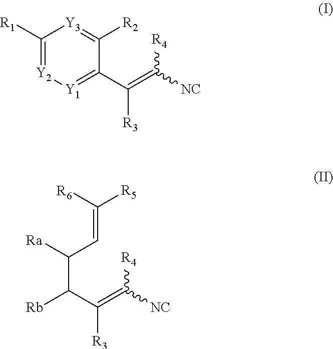

or a salt, solvate (or a diastereomer) or tautomer thereof, wherein:
Y₁, Y₂ and Y₃ are independently selected from C—R₁ or N;
each R₁ is independently selected from H, C₁ to C₆ alkyl, OH, OR, NHCOR, NHSO₂R, CONHR, CONHSO₂R, substituted or unsubstituted aryl, substituted or unsubstituted heteroaryl or R₇; and each R is independently selected from H, or C₁ to C₆ alkyl;
R₂ is selected from substituted or unsubstituted aryl, substituted or unsubstituted heteroaryl, or R₇;
R₃ and R₄ are independently selected from H, or C₁ to C₆ alkyl;
R₇ is a group of formula:

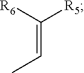

Rₐ and R_{b} are independently selected from H, or C₁ to C₆ alkyl; or Rₐ and R_{b} together with the atoms to which they are attached, form a substituted or unsubstituted 5 or 6 membered ring;
R₅ is selected from substituted or unsubstituted aryl or heteroaryl; and
R₆ is selected from H, or C₁ to C₆ alkyl.

The substituted or unsubstituted 5 or 6 membered ring may comprise a substituted or unsubstituted cyclyl or heterocyclyl ring, suitably a C₅₋₂₀ cyclyl or C₅₋₁₀ heterocyclyl.

The substituted or unsubstituted 5 or 6 membered ring may comprise a substituted or unsubstituted aryl or heteroaryl ring or a ring forming one ring of a fused ring structure, suitably wherein the substituted or unsubstituted 5 or 6 membered ring comprises a substituted or unsubstituted C₅₋₂₀ aryl or C₅₋₁₀ heteroaryl.

The substituted or unsubstituted 5 or 6 membered ring may be selected from pyrrolidine, pyrrole, pyridine, furan, thiophene, oxazole, isoxazole, isoxazine, oxadiazole (e.g. 1-oxa-2,3-diazolyl, 1-oxa-2,4-diazolyl, 1-oxa-2,5-diazolyl, 1-oxa-3,4-diazolyl), oxatriazole, thiazole, isothiazole, imidazole, pyrazole, pyridazine, pyrimidine, pyrazine, triazole (e.g. 1,2,4-triazole), triazine (e.g. 1,2,4-triazine), tetrazole, azaindole (e.g. 5-azaindole or 7-azaindole), azaindazole (e.g. 7-azaindolazole), azabenzimidazole (e.g. 5-azabenzimidazole), benzofuran, isobenzofuran, indole, quinoline, quinazoline, isoindole, indolizine, isoindoline, benzothiofuran, benzoxazole, benzisoxazole, benzothiazole, benzimidazole, indazole, benzodioxole, benzofurazan, benzothiadiazole, benzotriazole, purine (e.g., adenine, guanine), pyrrolo[1,2-a]pyrazine, pyrazolo[1,5-a]pyridine, 1H-pyrazolo[3,4-d]pyrimidine, pyrazolo[1,5-b]pyridazine, and pteridine.

The compound may be of formula (III):

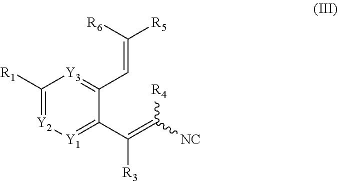

wherein R₁ is selected from H, C₁ to C₆ alkyl, OH, OR, NHCOR, NHSO₂R, CONHR, CONHSO₂R or substituted or unsubstituted aryl or substituted or unsubstituted heteroaryl;
Y₁, Y₂ and Y₃ are independently selected from C—R or N; and
each R is independently selected from H, or C₁ to C₆ alkyl.

The compound may be of formula (III):

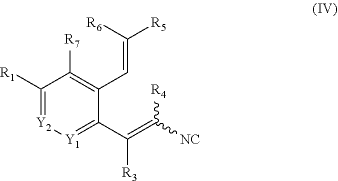

wherein R₆ and R₇ are independently selected from H, C₁ to C₆ alkyl, OH, or OR; or R₆ and R₇ together with the atoms to which they are attached form a substituted or unsubstituted 6 membered ring.

The compound may be of formula (V):

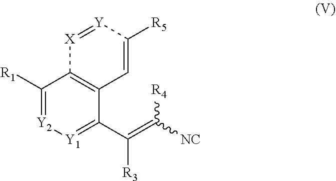

wherein the dotted lines to X and Y each independently indicate the optional presence of a bond; and X and Y are each independently selected from C(R)ₙ or N(R)ₘ; wherein n is 1 or 2 and m is 0 or 1 depending on the optional presence of a bond; with the proviso that at least one of the dotted lines to X and Y indicate the presence of a bond.

The compound may be of formula (VI):

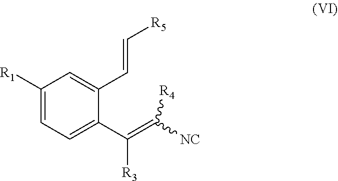

Suitably, R₅ may be substituted or unsubstituted aryl, pyridyl, pyrazyl, pyridazyl or pyrimidyl.

Suitably, R₃ and R₄ may each be H.

Suitably, each of Y₁, Y₂ and Y₃ may be each C—R₁.

Suitably, at least one R₁ is selected from OH, OR, NHCOR, NHSO₂R, CONHR, CONHSO₂R, wherein each R is independently selected from H, or C₁ to C₆ alkyl. More suitably, each of Y₁, Y₂ and Y₃ may each be C-Ria, wherein each Ria is independently selected from H, C₁ to C₆ alkyl, substituted or unsubstituted aryl, substituted or unsubstituted heteroaryl or R₇.

Suitably, the compound may be selected from compounds of formulae:

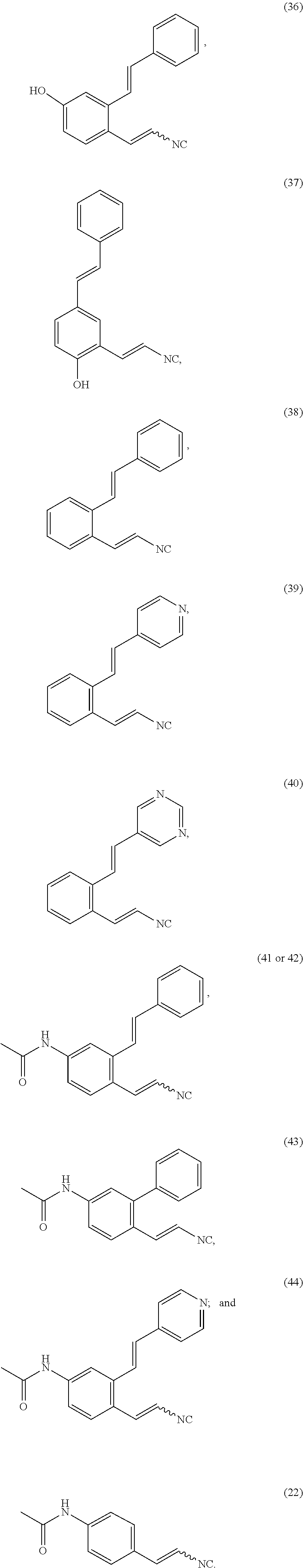

In a second aspect, there is provided a method of producing a vinyl isocyanide compound of the first aspect, the method comprising:
a) providing a phosphonate of formula (X):

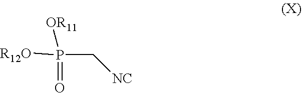

wherein R₁₁ and R₁₂ are independently selected from C₃ to C₅ alkyl, optionally independently selected from isopropyl, isobutyl, and t-butyl.
b) reacting the phosphonate with a carbonyl compound in the presence of base.

The carbonyl compound may be a compound of formula (XI) or (XII):

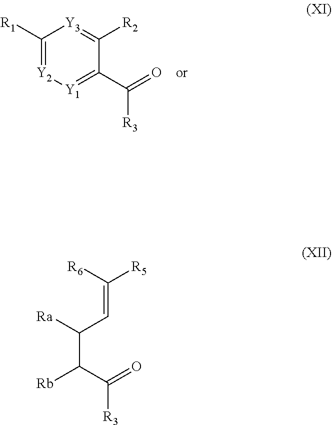

wherein, Y₁, Y₂ and Y₃ are independently selected from C—R₁ or N;
each R₁ is independently selected from H, C₁ to C₆ alkyl, OH, OR, NHCOR, NHSO₂R, CONHR, CONHSO₂R, substituted or unsubstituted aryl, substituted or unsubstituted heteroaryl or R₇; and each R is independently selected from H, or C₁ to C₆ alkyl;
R₂ is selected from substituted or unsubstituted aryl, substituted or unsubstituted heteroaryl, or R₇;
R₃ is selected from H, or C₁ to C₆ alkyl;
R₇ is a group of formula:

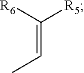

Rₐ and R_{b} are independently selected from H, or C₁ to C₆ alkyl; or Rₐ and R_{b} together with the atoms to which they are attached, form a substituted or unsubstituted 5 or 6 membered ring;
R₅ is selected from substituted or unsubstituted aryl or heteroaryl; and
R₆ is selected from H, or C₁ to C₆ alkyl.

The base may comprise a non-nucleophilic base, optionally a Li base, optionally a base selected from lithium bis(trimethylsilyl)amide (LHMDS), lithium tetramethylpiperidide (LiTMP), and lithium diisopropylamide (LDA).

Suitably, R₃ may be H.

Suitably, the phosphonate may be reacted with a carbonyl in the presence of base and THE as solvent.

In a third aspect, there is provided a reagent for use in the method of the third aspect, the reagent comprising a compound of formula (X):

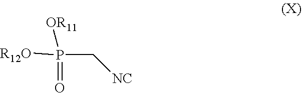

wherein R₁₁ and R₁₂ are independently selected from C₃ to C₅ alkyl, optionally independently selected from isopropyl, isobutyl, and t-butyl.

Compounds of formula (I) or (II) and salts and solvates thereof may be used as a medicament, especially for use in the treatment of an infectious disease, more particularly, for use in the treatment of a bacterial, fungal or protozoal disease. Where the compound is for treatment of a bacterial disease, the disease may be a disease caused by gram negative bacteria, or gram positive bacteria.

In a fourth aspect, there is accordingly provided a pharmaceutical composition comprising a compound of formula (I) or (II) and salts and solvates thereof and a pharmaceutically acceptable excipient, carrier or diluent.

Further particular and preferred aspects are set out in the accompanying independent and dependent claims. Features of the dependent claims may be combined with features of the independent claims as appropriate, and in combinations other than those explicitly set out in the claims, as supported by the description.

### Definitions

“Substituted”, when used in connection with a chemical substituent or moiety (e.g., an alkyl group), means that one or more hydrogen atoms of the substituent or moiety have been replaced with one or more non-hydrogen atoms or groups, provided that valence requirements are met and that a chemically stable compound results from the substitution.

“Optionally substituted” refers to a parent group which may be un-substituted or which may be substituted with one or more substituents. Suitably, unless otherwise specified, when optional substituents are present the optional substituted parent group comprises from one to three optional substituents. Where a group may be “optionally substituted with 1, 2 or 3 groups”, this means that the group may be substituted with 0, 1, 2 or 3 of the optional substituents. Suitably, the group is substituted with 1, 2 or 3 of the optional substituents. Where a group is “optionally substituted with one or two optional substituents”, this means that the group may be substituted with 0, 1 or 2 of the optional substituents. Suitably, the group may be optionally substituted with 0 or 1 optional substituents. In some aspects, suitably the group is not optionally substituted. In other aspects, suitably the group is substituted with 1 of the optional substituents.

Optional substituents may be selected from C₁₋₈ alkyl, C₂₋₇ alkenyl, C₂₋₇ alkynyl, C₁₋₁₂ alkoxy, C₅₋₂₀ aryl, C₃₋₁₀ cycloalkyl, C₃₋₁₀ cycloalkenyl, C₃₋₁₀ cycloalkynyl, C₃₋₂₀ heterocyclyl, C₃₋₂₀ heteroaryl, acetal, acyl, acylamido, acyloxy, amidino, amido, amino, aminocarbonyloxy, azido, carboxy, cyano, ether, formyl, guanidino, halo, hemiacetal, hemiketal, hydroxamic acid, hydroxyl, imidic acid, imino, ketal, nitro, nitroso, oxo, oxycarbonyl, oxycarboyloxy, sulfamino, sulfamyl, sulfate, sulfhydryl, sulfinamino, sulfinate, sulfino, sulfinyl, sulfinyloxy, sulfo, sulfonamido, sulfonamino, sulfonate, sulfonyl, sulfonyloxy, uredio groups. In some aspects, the optional substituents are 1, 2 or 3 optional substituents independently selected from OH, C₁₋₈ alkyl, OC₁₋₁₂ alkyl, and halogen. More suitably, the optional substituents are selected from OH, C₁₋₈ alkyl and OC₁₋₁₂ alkyl; more suitably, the optional substituents are selected from C₁₋₈ alkyl and OC₁₋₁₂ alkyl.

“Independently” or “Independently selected” is used in the context of statement that, for example, “each R₁₆, R₁₇ is independently H, C₁₋₈ alkyl, . . . ” and means that each instance of the functional group, e.g. R₁₆, is selected from the listed options independently of any other instance of R₁₆ or R₁₇ in the compound. Hence, for example, H may be selected for the first instance of R₁₆ in the compound; methyl may be selected for the next instance of R₁₆ in the compound; and ethyl may be selected for the first instance of R₁₇ in the compound.

C₁₋₈ alkyl: refers to straight chain and branched saturated hydrocarbon groups, generally having from 1 to 8 carbon atoms; suitably a C₁₋₇ alkyl; suitably a C₁₋₆ alkyl; suitably a C₁₋₅ alkyl; more suitably a C₁₋₄ alkyl; more suitably a C₁₋₃ alkyl. Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, n-heptyl, n-octyl and the like.

“Alkylene” refers to a divalent radical derived from an alkane which may be a straight chain or branched, as exemplified by —CH₂CH₂CH₂CH₂—. The alkylene may have the number of carbons as discussed above for alkyl groups.

“C₆₋₂₆ aralkyl” refers to an arylalkyl group having 6 to 26 carbon atoms and comprising an alkyl group substituted with an aryl group. Suitably the alkyl group is a C₁₋₆ alkyl group and the aryl group is phenyl. Examples of C₆₋₂₆ aralkyl include benzyl and phenethyl. In some cases the C₆₋₂₆ aralkyl group may be optionally substituted and an example of an optionally substituted C₆₋₂₆ aralkyl group is 4-methoxylbenzyl.

“C₅₋₂₀ Aryl”: refers to fully unsaturated monocyclic, bicyclic and polycyclic aromatic hydrocarbons having at least one aromatic ring and having a specified number of carbon atoms that comprise their ring members (e.g., C₅₋₂₀ aryl refers to an aryl group having from 5 to 20 carbon atoms as ring members). The aryl group may be attached to a parent group or to a substrate at any ring atom and may include one or more non-hydrogen substituents unless such attachment or substitution would violate valence requirements. Suitably, a C₅₋₂₀ aryl is selected from a C₆₋₁₄ aryl, or a C₆₋₁₂ aryl, more suitably, a C₆₋₁₀ aryl. Examples of aryl groups include phenyl.

“Arylene” refers to a divalent radical derived from an aryl group, e.g. —C₆H₄— which is the arylene derived from phenyl.

Halogen or halo: refers to a group selected from F, Cl, Br, and I. Suitably, the halogen or halo is F or Cl. In some aspects, suitably, the halogen is F. In other aspects, suitably the halogen is Cl.

“C₅₋₁₀ heteroaryl” or “5- to 10-membered heteroaryl”: refers to unsaturated monocyclic or bicyclic aromatic groups comprising from 5 to 10 ring atoms, whether carbon or heteroatoms, of which from 1 to 5 are ring heteroatoms. Suitably, any monocyclic heteroaryl ring has from 5 to 6 ring atoms and from 1 to 3 ring heteroatoms. Suitably each ring heteroatom is independently selected from nitrogen, oxygen, and sulfur. The bicyclic rings include fused ring systems and, in particular, include bicyclic groups in which a monocyclic heterocycle comprising 5 ring atoms is fused to a benzene ring. The heteroaryl group may be attached to a parent group or to a substrate at any ring atom and may include one or more non-hydrogen substituents unless such attachment or substitution would violate valence requirements or result in a chemically unstable compound.

Examples of monocyclic heteroaryl groups include, but are not limited to, those derived from:
N₁: pyrrole, pyridine;
O₁: furan;
S₁: thiophene;
N₁O₁: oxazole, isoxazole, isoxazine;
N₂O₁: oxadiazole (e.g. 1-oxa-2,3-diazolyl, 1-oxa-2,4-diazolyl, 1-oxa-2,5-diazolyl, 1-oxa-3,4-diazolyl);
N₃O₁: oxatriazole;
N₁S₁: thiazole, isothiazole;
N₂: imidazole, pyrazole, pyridazine, pyrimidine, pyrazine;
N₃: triazole, triazine; and,
N₄: tetrazole.

Examples of heteroaryl which comprise fused rings, include, but are not limited to, those derived from:
O₁: benzofuran, isobenzofuran;
N₁: indole, isoindole, indolizine, isoindoline;
S₁: benzothiofuran;
N₁O₁: benzoxazole, benzisoxazole;
N₁S₁: benzothiazole;
N₂: benzimidazole, indazole;
O₂: benzodioxole;
N₂O₁: benzofurazan;
N₂S₁: benzothiadiazole;
N₃: benzotriazole; and
N₄: purine (e.g., adenine, guanine), pteridine;

“Heteroarylene” refers to a divalent radical derived from a heteroaryl group (such as those described above) as exemplified by pyridinyl —[C₅H₃N]—. Heteroarylenes may be monocyclic, bicyclic, or tricyclic ring systems. Representative heteroarylenes, are not limited to, but may be selected from triazolylene, tetrazolylene, oxadiazolylene, pyridylene, furylene, benzofuranylene, thiophenylene, benzothiophenylene, quinolinylene, pyrrolylene, indolylene, oxazolylene, benzoxazolylene, imidazolylene, benzimidazolylene, thiazolylene, benzothiazolylene, isoxazolylene, pyrazolylene, isothiazolylene, pyridazinylene, pyrimidinylene, pyrazinylene, triazinylene, cinnolinylene, phthalazinylene, quinazolinylene, pyrimidylene, azepinylene, oxepinylene, and quinoxalinylene. Heteroarylenes are optionally substituted.

“C₆₋₁₆ heteroarylalkyl” refers to an alkyl group substituted with a heteroaryl group. Suitably the alkyl is a C₁₋₆ alkyl group and the heteroaryl group is C₅₋₁₀ heteroaryl as defined above. Examples of C₆₋₁₆ heteroarylalkyl groups include pyrrol-2-ylmethyl, pyrrol-3-ylmethyl, pyrrol-4-ylmethyl, pyrrol-3-ylethyl, pyrrol-4-ylethyl, imidazol-2-ylmethyl, imidazol-4-ylmethyl, imidazol-4-ylethyl, thiophen-3-ylmethyl, furan-3-ylmethyl, pyridin-2-ylmethyl, pyridin-2-ylethyl, thiazol-2-ylmethyl, thiazol-4-ylmethyl, thiazol-2-ylethyl, pyrimidin-2-ylpropyl, and the like.

“C₃₋₂₀ heterocyclyl”: refers to saturated or partially unsaturated monocyclic, bicyclic or polycyclic groups having ring atoms composed of 3 to 20 ring atoms, whether carbon atoms or heteroatoms, of which from 0 to 10 are ring heteroatoms. Suitably, each ring has from 3 to 8 ring atoms and from 1 to 4 ring heteroatoms (e.g., suitably C₃₋₅ heterocyclyl refers to a heterocyclyl group having 3 to 5 ring atoms and 1 to 4 heteroatoms as ring members). The ring heteroatoms are independently selected from nitrogen, oxygen, and sulphur.

As with bicyclic cycloalkyl groups, bicyclic heterocyclyl groups may include isolated rings, spiro rings, fused rings, and bridged rings. The heterocyclyl group may be attached to a parent group or to a substrate at any ring atom and may include one or more non-hydrogen substituents unless such attachment or substitution would violate valence requirements or result in a chemically unstable compound.

Examples of monocyclic heterocyclyl groups include, but are not limited to, those derived from:
N₁: aziridine, azetidine, pyrrolidine, pyrroline, 2H-pyrrole or 3H-pyrrole, piperidine, dihydropyridine, tetrahydropyridine, azepine;
O₁: oxirane, oxetane, tetrahydrofuran, dihydrofuran, tetrahydropyran, dihydropyran, pyran, oxepin;
S₁: thiirane, thietane, tetrahydrothiophene, tetrahydrothiopyran, thiepane;
O₂: dioxoiane, dioxane, and dioxepane;
O₃: trioxane;
N₂: imidazoiidine, pyrazolidine, imidazoline, pyrazoline, piperazine:
N₁O₁: tetrahydrooxazole, dihydrooxazole, tetrahydroisoxazole, dihydroisoxazole, morpholine, tetrahydrooxazine, dihydrooxazine, oxazine;
N₁S₁: thiazoline, thiazolidine, thiomorpholine;
N₂O₁: oxadiazine;
O₁S₁: oxathiole and oxathiane (thioxane); and
N₁O₁S₁: oxathiazine.

Examples of substituted monocyclic heterocyclyl groups include those derived from saccharides, in cyclic form, for example, furanoses, such as arabinofuranose, lyxofuranose, ribofuranose, and xylofuranse, and pyranoses, such as aliopyranose, altropyranose, glucopyranose, mannopyranose, gulopyranose, idopyranose, galactopyranose, and talopyranose.

“Drug”, “drug substance”, “active pharmaceutical ingredient”, and the like, refer to a compound (e.g., compounds of Formula (I) and compounds specifically named above) that may be used for treating a subject in need of treatment.

“Excipient” refers to any substance that may influence the bioavailability of a drug but is otherwise pharmacologically inactive.

The term “or pharmaceutically acceptable salts, solvates, tautomers, stereoisomers or mixtures thereof” means that pharmaceutically acceptable salt, solvate, tautomeric, stereoisomeric forms of the shown structure are also included. Mixtures thereof means that mixture of these forms may be present, for example, the compounds of the invention may include both a tautomeric form and a pharmaceutically acceptable salt.

“Pharmaceutically acceptable” substances refers to those substances which are within the scope of sound medical judgment suitable for use in contact with the tissues of subjects without undue toxicity, irritation, allergic response, and the like, commensurate with a reasonable benefit-to-risk ratio, and effective for their intended use.

“Pharmaceutical composition” refers to the combination of one or more drug substances and one or more excipients.

As used herein, “solvate” refers to a complex of variable stoichiometry formed by a solute and a solvent. Pharmaceutically acceptable solvates may be formed for crystalline compounds wherein solvent molecules are incorporated into the crystalline lattice during crystallization. The incorporated solvent molecules can be water molecules or non-aqueous molecules, such as but not limited to, ethanol, isopropanol, dimethyl sulfoxide, acetic acid, ethanolamine, and ethyl acetate molecules.

The term “subject” as used herein refers to a human or non-human mammal. Examples of non-human mammals include livestock animals such as sheep, horses, cows, pigs, goats, rabbits and deer; and companion animals such as cats, dogs, rodents, and horses.

“Therapeutically effective amount” of a drug refers to the quantity of the drug or composition that is effective in treating a subject and thus producing the desired therapeutic, ameliorative, inhibitory or preventative effect. The therapeutically effective amount may depend on the weight and age of the subject and the route of administration, among other things.

“Treating” refers to reversing, alleviating, inhibiting the progress of, or preventing a disorder, disease or condition to which such term applies, or to reversing, alleviating, inhibiting the progress of, or preventing one or more symptoms of such disorder, disease or condition.

“Treatment” refers to the act of “treating”, as defined immediately above.

As used herein the term “comprising” means “including at least in part of” and is meant to be inclusive or open ended. When interpreting each statement in this specification that includes the term “comprising”, features, elements and/or steps other than that or those prefaced by the term may also be present. Related terms such as “comprise” and “comprises” are to be interpreted in the same manner.

The term “consisting essentially of” limits the scope of a claim to the specified materials or steps “and those that do not materially affect the basic and novel characteristic(s)” of the claimed invention. When the phrase “consisting essentially of” appears in a clause of the body of a claim, rather than immediately following the preamble, it limits only the element set forth in that clause.

The term “consisting of” excludes any element, step, or ingredient not specified in the claim; “consisting of” defined as “closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole. It should be understood that while various embodiments in the specification are presented using “comprising” language, under various circumstances, a related embodiment is also described using “consisting essentially of” or “consisting of” language.

### BRIEF DESCRIPTION OF THE DRAWINGS

Embodiments of the present invention will now be described further, with reference to the accompanying drawings, in which:

FIG. 1: Average increase in mass of Manduca Sexta injected with 10 μL at 1 mg/mL by selected compounds according to the invention plus PBS (+) and 1% NaN₃ (−) controls. The mass of the Manduca were recorded 24 and 72 hours after inoculation (n=5).

FIG. 2: The prevention (left) and attenuation (right) of MRSA 252 biofilms by selected compounds according to the invention. Graphs shows (left to right) non-bacterial control; MRSA 252 without antimicrobial; compounds 2; 13; 14; 22; 36; 41.

FIG. 3: The prevention of high biofilm forming S. aureus strains at 50 μg/mL. AS 68 (a), AS 140 (b) and various concentrations of compounds using S. aureus AS 68 (c).

FIG. 4: Removal of mature AS 68 biofilms for by selected compounds according to the invention at 50 μg/mL (left); at various concentrations for compound 41 (right).

FIG. 5: Time-kill assays for compound 41 on late exponential phase MRSA 252.

FIG. 6: SEM images of MRSA 252 bacteria (a) and MRSA 252 cells after 8 hours addition of compound 41 (b and c).

FIG. 7: Changes in the bacterial cell membrane were demonstrated by fluorescent microscopy when MRSA 252 cells were treated with compound 41 (right). Control MRSA 252 cells with no antibiotic added (left).

FIG. 8: Percentage membrane potential of MRSA cells subjected to a range of compounds for 180 minutes.

FIG. 9: Mean percentage K⁺ ions remaining in MRSA cells subjected to a CTAB and compound 41 for 60 minutes.

FIG. 10: MIC values following the serial passaging of MRSA 252 in the presence of compound 36 and ofloxacin.

FIG. 11: NMR data for a) Compound 41 and b) compound 41 after 6 month's storage in organic solvent.

FIG. 12: NMR data for compound 39 in the presence of glutathione at various set intervals.

FIG. 13: NMR data for Glutathione in DMSO-D₆.

FIG. 14: NMR data for Compound 39 (30 minutes after glutathione addition).

FIG. 15: NMR data for Compound 39 (5 hours after glutathione addition).

FIG. 16: NMR data for Compound 39 (24 hours after glutathione addition).

FIG. 17: Plotted MIC data (Origin Lab) for compound 41 before and after cysteine addition.

FIG. 18: NMR data for compound 41 (a) before and (b) after 1M acetic acid exposure for 4 hours.

### DESCRIPTION OF THE EMBODIMENTS

The library of vinyl isocyanide compounds synthesized in this work were shown to possess good activity against planktonic Staphylococcus aureus. Further biological studies revealed their excellent anti-biofilm properties, with complete prevention of biofilm growth at sub-MIC concentrations (as low as 1 μg/mL, the lowest concentration tested). These compounds also demonstrate no systemic toxicity issues at concentrations exceeding 500 μg/mL using both Galleria mellonella and Manduca sexta systemic infection models. Despite the cell membrane being identified as the target site of action for these compounds, selected ‘lead compounds’ have shown a remarkable degree of selectivity, with no significant cytotoxicity against human embryonic kidney cells (HEK 293 cells), and no hemolysis detected at 32 p/mL (the highest concentration tested). Unlike typical organic based antibiotic compounds, further in vitro studies have shown that bacteria may face difficulties in conferring resistance to these novel vinyl isocyanide compounds. Following 18 cycles of serial passaging, no detectable resistance was identified further suggesting their high potential for use as new antibiotics.

A new diisopropyl isocyanomethylphosphonate reagent has been shown to undergo highly diastereoselective Horner-Wadsworth-Emmons (HWE) reactions with cyclic and acyclic aldehydes to afford vinyl isocyanides with excellent (E)-selectivities. A series of experimental studies have been carried out to explain how the isopropyl phosphonate reagent affords significantly higher levels of (E)-diastereocontrol than its corresponding ethyl derivative. Stereoselectivity in these HWE reactions is determined in the first irreversible addition step with the absence/presence of non-classical alkoxide-C—H interactions in the transition state of the isopropyl HWE reagent responsible for its greater (E)-selectivity. This new isopropyl HWE reagent has been used as a key reaction for the first 7-step synthesis of Byelyankacin and its aglycone which are vinyl isocyanide natural products produced by Gram-negative bacterial species that live in the guts of entomopathogenic nematodes that prey on insect larvae in the soil. Access to this vinyl cyanide enabled us to demonstrate that Byelyankacin is a multipotent natural product, acting both as an inhibitor of insect phenol-oxidases to produce melanin as part of an insect's immune response, and as an antibiotic to prevent competing Gram-positive bacteria from consuming the insect cadaver. Byelyankacin and its aglycone have been shown to demonstrate good antibiotic activity against clinically relevant methicillin resistant Staphylococcus aureus strains, thus confirming the potential of vinyl isocyanides as antibiotics for the treatment of clinically relevant bacterial infections.

Synthesis and testing of 4-phenol vinyl isocyanide (2) demonstrated its moderate activity against S. aureus planktonic bacteria (MRSA 252: 90 μg/mL and MSSA 476: 100 μg/mL). Subsequently, it was decided to conduct a small structure-activity relationship (SAR) study on this particular compound (2), in order to identify the moieties that are essential for its biological activity. The synthesis, SAR study and the biological performance of the library of compounds synthesized are detailed herein.

To investigate whether these compounds displayed a broad range of antibiotic activity, all compounds synthesized were tested against three clinically relevant Gram-positive pathogen S. aureus strains (MRSA 252, MSSA 476 and MSSA 15981) and two Gram-negative species: Pseudomonas aeruginosa PAO1 and Escherichia co/i DH5a using the semi-quantitative disc diffusion assay at a set compound concentration of 500 μg/mL (Table 3). The results showed that the vast majority of these compounds were specifically active against S. aureus with large zones of inhibition observed. In general, only small or no zones of inhibition were observed for the compounds against both Gram negative species. As such, the minimum inhibition concentration (MICs) of biologically active compounds were further assessed using the broth dilution method against the S. aureus strains; MRSA 252 and MSSA 476 (Table 1). The results of both assays enabled key structural elements and functional groups required for the compounds to retain their antibiotic activity to be identified.

### Structure Activity Relationship (SAR)

### Isocyanide Group

Disc diffusion assays revealed that replacement of the isocyanide group with its cyanide derivative (compound 3) resulted in the complete loss of biological activity. The lack of any noticeable zone of inhibition against all five strains tested (Table 3), demonstrates that 3 has no antibiotic activity. As such, it can be rationalized that the isocyanide moiety must play an essential role in binding to the target site of action in the bacteria that confers antibiotic activity.

### Vinyl Group

The significance of the double bond in 4-phenol vinyl isocyanide (2) was next evaluated. Saturation of this double bond (4) led to the complete loss of activity against all five strains, demonstrating the requirement of this functional group. During this part of the SAR study 4-isocyanophenol (5) was also prepared.

### Other Compounds

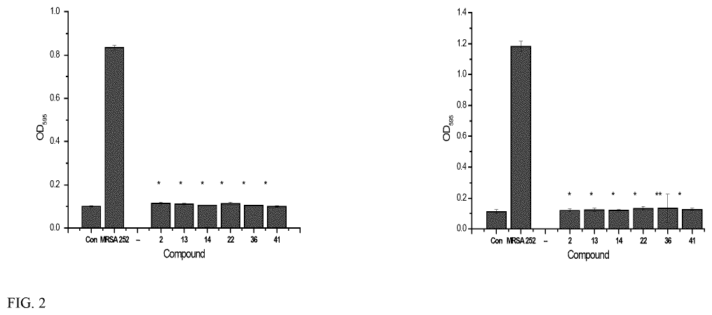

Other compounds (36, 37 and 38) (FIG. 2) retained antibiotic activity in disc diffusion assays, with large zones of inhibitions observed (Table 3).

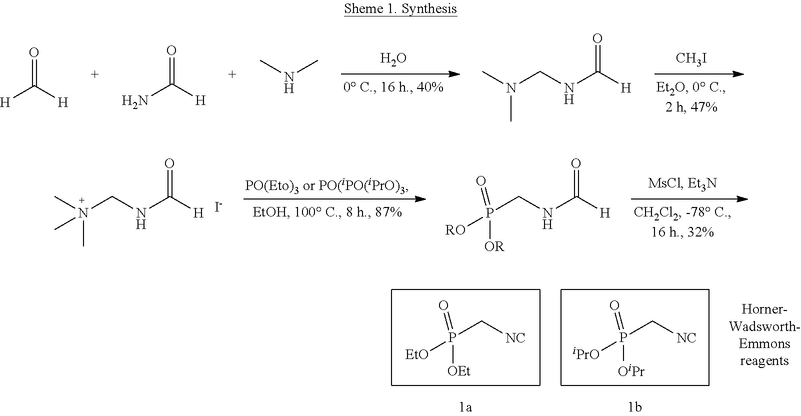

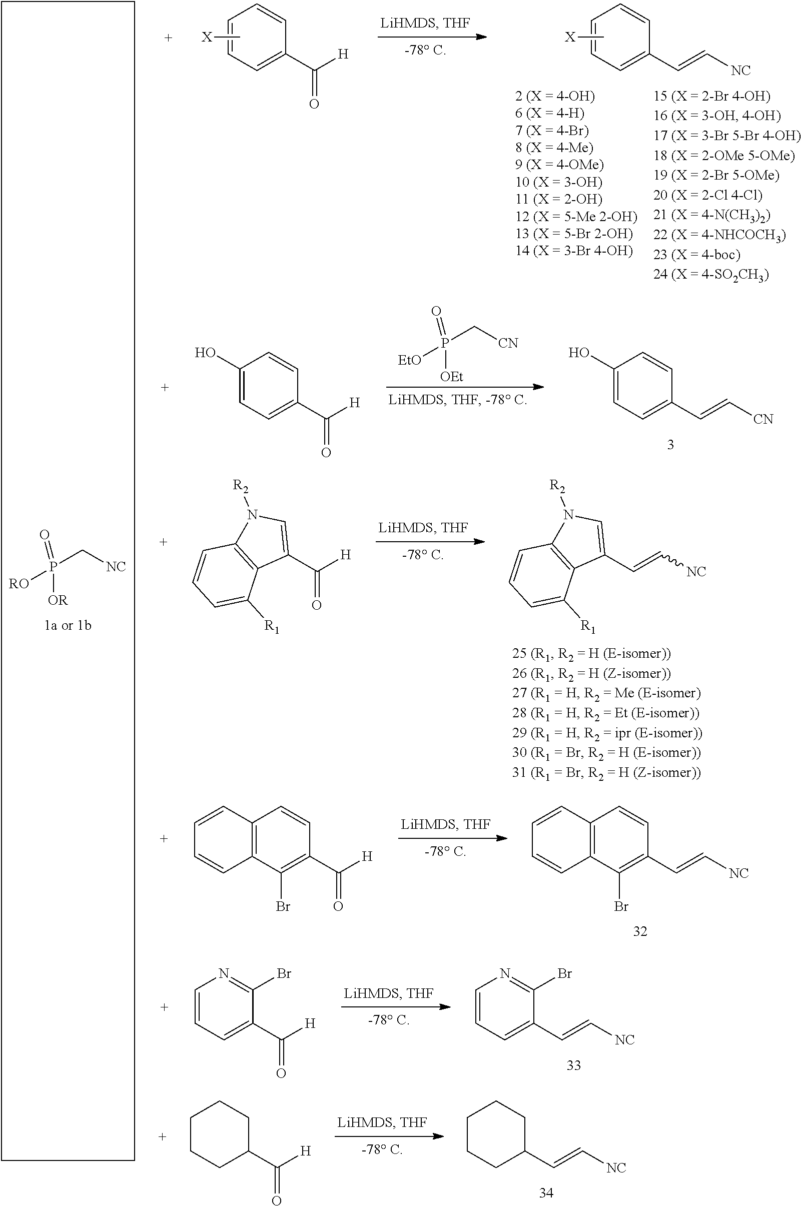

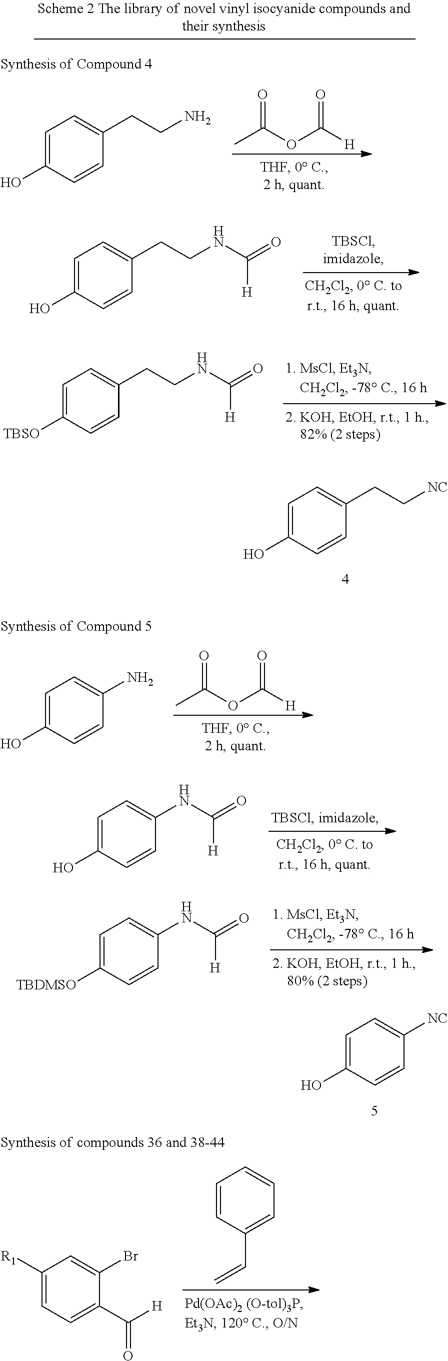

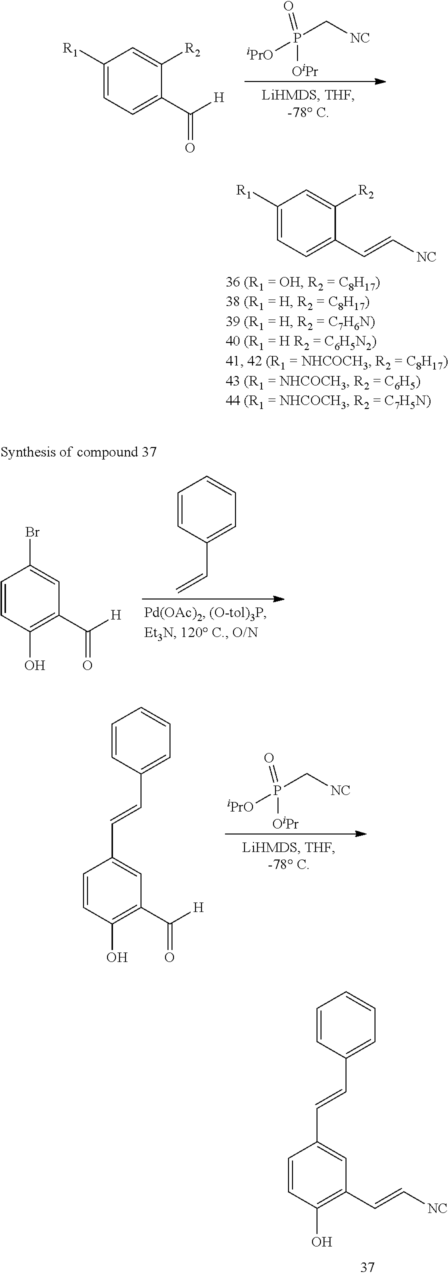

Evaluation of these compounds by MIC indicated their increased potency against S. aureus with a significant reduction in the MIC shown for each compound (36 and 38). Comparing compounds 36 and 37, it can be concluded that the fragment in the ortho-position improves the antimicrobial performance (Table 1). Surprisingly compound 38, which does not contain the hydroxyl group in the para-position was extremely active against MRSA 252: 5 μg/mL and MSSA 476: 3 μg/mL.

However, the addition of styrene reduces the water solubility of the compounds, as demonstrated by the partial insolubility of the compounds 36-38 in 2% DMSO/water. To improve water solubility compounds 39 and 40 were synthesized, in which the styrene analogue in the ortho-position was replaced with 4-vinyl pyridine and 2-vinyl pyrazine respectively. It was believed that these two compounds could still contain the essential functionalities required to retain their antimicrobial activity, but had improved aqueous solubility properties, important for their potential in vivo application. Compound 39 showed excellent activity against both MRSA 252: 12 μg/mL and MSSA 476: 15 μg/mL whilst also retaining its activity against the Gram negative P. aeruginosa and E. coli (Table 3). Although antibiotic activity was retained for compound 40, there was a slight decrease in its activity against both MRSA 252 (32 μg/mL) and MSSA 476 (26 μg/mL) (Table 1).

Compound 41 was synthesized: replacing the hydroxyl functionality in compound 36 with an amide functionality. Compound 41 showed excellent activity against all three S. aureus strains, as well as P. aeruginosa PAO1 in disc diffusion assays: MIC values (MRSA 252: 6 μg/mL and MSSA 476: 8 μg/mL). In synthesizing compound 41, it was possible to separate the E- and Z-isomeric forms by column chromatography. Independent MIC results obtained for each isomer revealed the E-isomer (41) was much more potent than its Z-counterpart (42), suggesting the E-isomer in these particular ‘second generation’ compounds is the favored orientation to maximize the interaction with the target site of action. To demonstrate the importance of the second vinylic bond in these compounds, compound 43 was synthesized (which lacked the vinylic moiety). Unsurprising to us, there was an increase in the obtained MIC against both MRSA 252 and MSSA 476, showing the importance of this moiety. Finally, to further improve the solubility of these second-generation compounds, compound 44 was synthesized, containing the more polar and potentially protonated pyridine fragment. Despite demonstrating antibiotic activity against four of the strains in disc diffusion assay, the MIC of 44 was shown to increase against both S. aureus strains (MRSA 252: 35 μg/mL and MSSA 476: 46 μg/mL.

### 2. Systemic Toxicity

Identifying a novel compound with antibiotic activity is just the start of a lengthy period in the antibiotic drug discovery process.⁽²³⁾ Many compounds in this process fail at selectively targeting pathogenic bacterial cells over eukaryotic cells, and are thus inadequate for use as antibiotics. Due in large to its isomeric nature to cyanide, the isocyanide functionality is often believed to possess the same levels of toxicity, and hence we were aware of the problems that may be associated with this essential moiety.⁽²⁴⁾ Consequently, to determine if the compounds showed systemic toxicity to a living organism, in vivo systemic toxicity studies using Greater Wax Moth larvae (Galleria mellonella) were performed. Evaluation of novel compound toxicity in mammalian models such as mice and rats is costly, time consuming, and require full ethical considerations.⁽²⁵⁾ In more recent times there has been an upsurge in the use of invertebrate models of infection to determine toxicity since a number of these invertebrates share many common features as that of the mammalian innate immune system.⁽²⁵⁾ Promisingly, apart from compound 5, this study has shown that all compounds (2, 6-44) appear to be non-toxic, with survival rates of Galleria equivalent to that given by the Phosphate Buffered Saline (PBS) control. Excluding compound 5, injection of 10 μL of the compounds at concentrations exceeding 500 μg/mL, produced no negative effects on the Galleria survival, as evidenced by the high survival rates, Table 4 (relative to the control). This would indicate that these novel set of compounds do not exhibit detrimental systemic toxicity in vivo, further detailing their potential use as antibiotics.

TABLE 1
MIC values obtained for active compounds
against MRSA 252 and MSSA 476.

Bacterial Strains

| Compound | MRSA 252 | MSSA 476 |
|---|---|---|
| Number | (μg/mL) | (μg/mL) |
| 2 | 90 | 100 |
| 5 | 26 | 23 |
| 10 | 137 | 300 |
| 11 | 250 | 350 |
| 12 | 123 | 187 |
| 13 | 35 | 145 |
| 14 | 44 | 242 |
| 15 | 185 | 200 |
| 16 | 116 | 400 |
| 17 | 157 | 176 |
| 18 | 117 | 210 |
| 22 | 55 | 172 |
| 23 | 35 | 164 |
| 24 | 152 | 291 |
| 25 | 61 | 166 |
| 26 | 20 | 222 |
| 28 | 56 | 144 |
| 29 | 63 | 90 |
| 30 | 214 | 350 |
| 31 | / | 361 |
| 36 | 15 | 12 |
| 37 | 105 | 120 |
| 38 | 5 | 3 |
| 39 | 12 | 15 |
| 40 | 32 | 26 |
| 41 | 6 | 8 |
| 42 | 110 | 45 |
| 43 | 32 | 26 |
| 44 | 35 | 46 |

At this point in the drug discovery program, it was decided to select a small number of compounds to conduct further microbiological and toxicological evaluation (Scheme 3). The compounds selected for further analysis were based on their relative ease of synthesis, disc diffusion assay data, the obtained MICs, as well as their low systemic toxicity. The compounds selected for further analysis were:

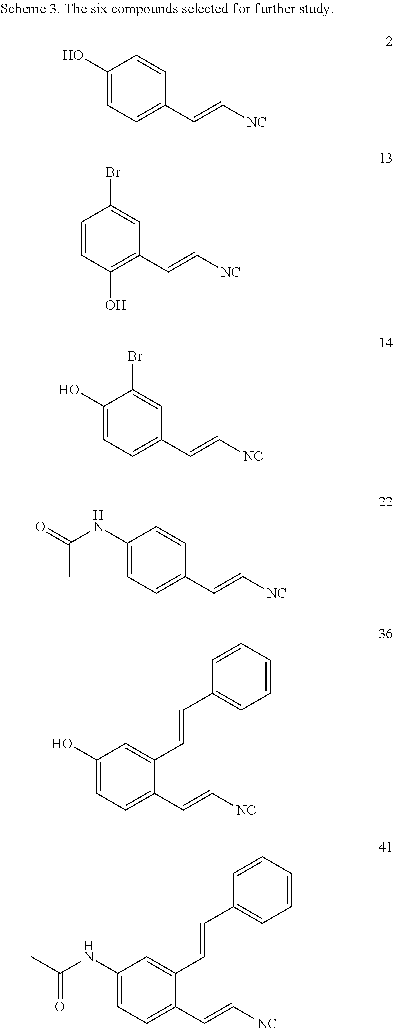

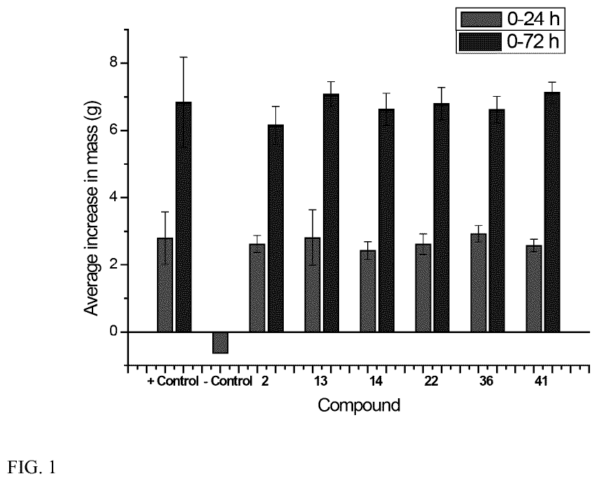

The use of Galleria for evaluating systemic toxicity study provided a straightforward method for screening any immediate toxicity issues associated with the lead compounds. One problem encountered with this model is the complications of obtaining quantitative results since only live/dead outcomes can be recorded. As an alternative and quantitative approach for systemic toxicity, it was decided to use a new model using Manduca sexta as the subject, replacing the much smaller and difficult to handle, Galleria mellonella. The Manduca sexta assay allows for the mass change of the larvae to be measured over hours/days, thus giving information not only on survival but also on the organisms' fitness⁽²⁶⁾ Promisingly, the 72 hour assay performed at concentrations of 1 mg/mL on the selected lead compounds gave identical results to those obtained using the Galleria infection model, indicating no apparent toxicity issues for the compounds. The increase in mass of the Manduca injected with the lead compounds was comparable to those injected with the positive control (PBS) (FIG. 1). As a negative control, the Manduca were injected with a 1% solution of NaN₃. After 24 hours there was a significant decrease in mass in comparison to the increase in mass demonstrated by the larvae injected with the lead antibiotic compounds. The Manduca injected with NaN₃ looked extremely unhealthy with signs of induced vomiting and diarrhea, and by 48 hours the negative control Manduca had all died. Those Manduca injected with the lead antibiotics remained healthy demonstrated by further gains in body mass.

3. Staphylococcus aureus Strain Study

Disc diffusion assays indicated that the library of compounds synthesized may be selective at inhibiting Gram positive bacteria, specifically S. aureus. At this point in the study it was decided to perform a disc diffusion assay study on 50 S. aureus strains. This was undertaken to demonstrate that the lead compounds can inhibit a large number of S. aureus strains both methicillin-resistant and methicillin-susceptible. Moreover, by employing a range of mutant strains of S. aureus with specific gene-deletions, it was believed that if particular strains were resistant to the lead compounds, it could provide insights on the likely protein target of the lead antibiotics. However, all six of the compounds utilized in this study were able to inhibit growth of all 50 strains of S. aureus assayed at the chosen concentration of 500 μg/mL concentration (Table 5).

### 4. Biofilm Study

The potential of the lead compounds to both inhibit S. aureus biofilm growth and to disrupt more established biofilms was next investigated. Since previous studies had determined each compounds MIC against planktonic MRSA bacteria, it was hypothesized that a compound concentration of 50 μg/mL (at or above the MIC of all lead compounds except 2) was a reasonable concentration to begin the study.

Biofilm biomass was estimated by crystal violet staining. Amazingly, S. aureus MRSA 252 biofilm growth was completely retarded by all lead compounds, suggesting they possess good biofilm prevention properties at 50 μg/mL. Typically the MIC to prevent and/or to eradicate bacterial biofilms can be up to 1000× greater than the planktonic MIC.⁽⁵⁾ It was thus encouraging that the lead compounds prevented biofilm formation around their planktonic MIC concentrations. Surprisingly, compound 2 also entirely prevented biofilm formation at approximately half its planktonic MIC (FIG. 2). Subsequently we wanted to investigate whether the selected compounds could eliminate mature S. aureus biofilms. For each compound there was a significant attenuation in the biofilm biomass as measured by crystal violet staining with complete removal of the biofilms at 50 μg/mL.

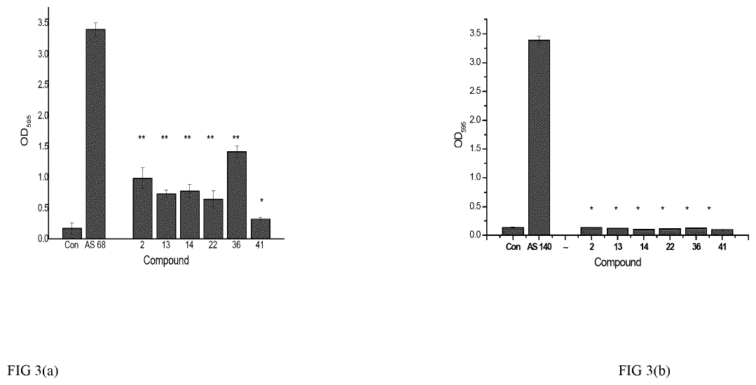

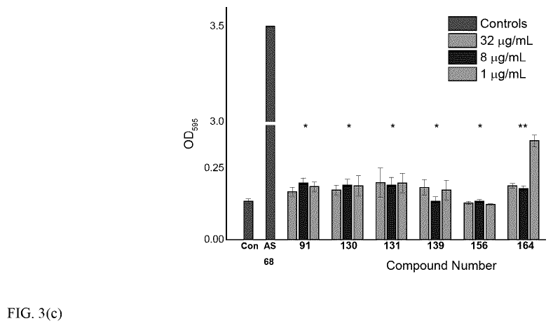

Based on these studies the biofilm prevention and eradication assays were repeated using strains of S. aureus which have a high propensity to form biofilms. The Asarm (AS) 68 and AS 140 strains selected for this study have mutations in their agr quorum-sensing systems known to improve biofilm development.⁽²⁷⁾ In this study, the lead compounds demonstrated complete prevention of the S. aureus biofilms (FIG. 3). All lead compounds were able to completely inhibit biofilm growth at 50 μg/mL. The MIC required to completely prevent the biofilm formation for the AS 68 MRSA strain was then established to be lower than 1 μg/mL for all lead compounds against S. aureus AS 68 (FIG. 3).

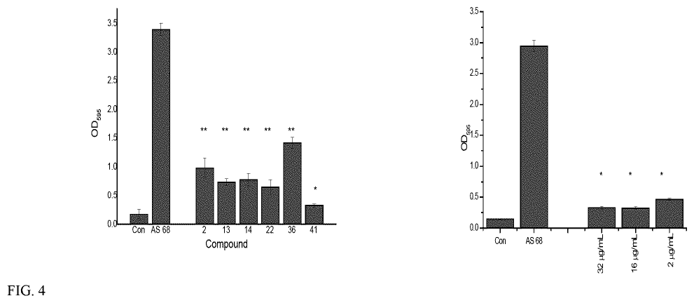

The attenuation of biomass by the lead compounds in the 24 hour old biofilms was measured by crystal violet stain assay. All compounds, and most notably 41 showed a significant attenuation in biomass of the S. aureus AS 68 biofilm, although not 100% eradication (FIG. 4). Further assays were performed to study the biofilm attenuation properties of compound 41. At concentrations, as low as 2 μg/ml (the lowest concentration tested), there was still an 86% reduction of the biofilm depth (FIG. 4).

### 5. Cytotoxicity

The cytotoxicity studies were performed by CO-ADD (The Community for Open Antimicrobial Drug Discovery), funded by the Wellcome trust (UK) and the University of Queensland, Australia.⁽²⁸⁾ In this study, the compounds were screened against a human embryonic kidney cell line, HEK293, at a set concentration of 32 μg/mL. At this particular concentration, (32× the concentration shown to prevent biofilm formation in some cases) the lead compounds (2, 13, 14, 22, 36, 41) showed no toxicity towards the HEK293 cell line, further indicating the potential use of these lead compounds as antibiotics (Table 6).

A problem often encountered by emerging drugs is hemolysis, also known as drug-induced immune hemolytic anemia. This can result in the premature rupturing of healthy red blood cells, causing a multitude of side-effects, including shortness of breath and dizziness to blood clots and heart failure.⁽²⁹⁾ Hemolysis assays conducted by the CO-ADD team showed HC₁₀ values to be >32 μg/mL (the highest concentration tested) (Table 6), suggesting that the lead compounds do not possess a significant degree of hemolytic activity at concentrations below 32 μg/mL.

### 6. Stability Assays

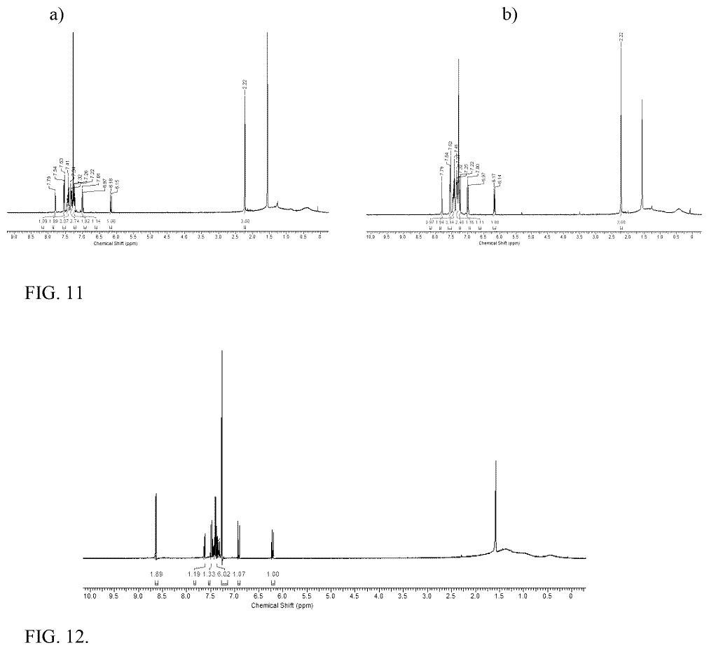

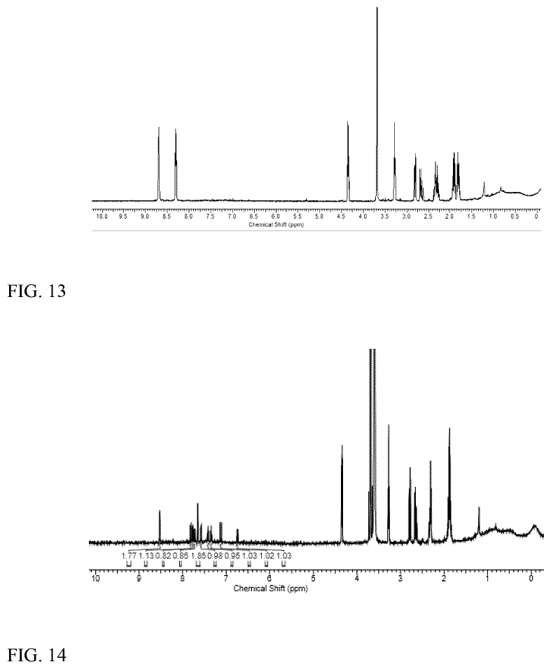

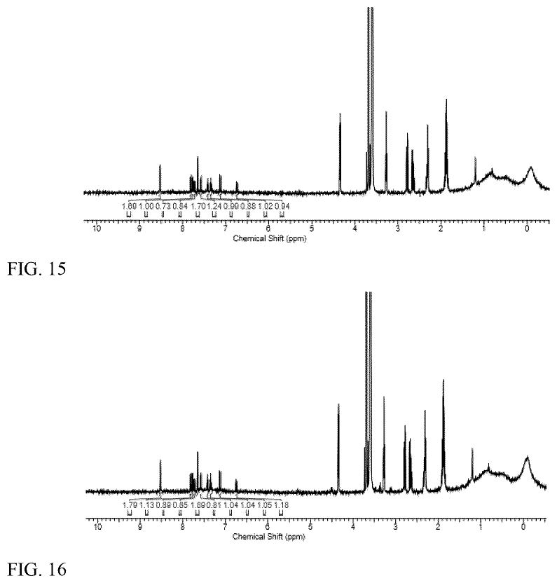

It is well known that compounds bearing an isocyanide group are usually highly reactive, capable of reacting with electrophiles, nucleophiles and radicals.⁽³⁰⁾ As a result, it was important to identify any stability issues which might be encountered by the lead compounds, both in storage and in the presence of glutathione and cysteine. The compounds are stable in organic solvents for over 6 months as demonstrated NMR (FIG. 11). Another common feature of the lead compounds is the vinyl fragment. It was hypothesized that the presence of glutathione and cysteine found in eukaryotic cells could potentially interact with the electrophilic alkene group in the compounds, thus shutting down their antibiotic activity. To probe this potentially detrimental in vivo process, compound 39 was subjected to glutathione for up to 24 hours whilst compound 41 was added to cysteine for the same length of time. NMR studies conducted after the elapsed time confirmed no change in the structure for compound 39 following exposure to glutathione. Furthermore, MIC values for 41 remained unaffected in the presence of cysteine, confirming the stability of our compounds in the presence of both cysteine and glutathione (FIGS. 12-16 and 17).

Also, well documented in the literature, is the hydrolysis of isocyanide compounds to their corresponding formamide in acidic conditions. Due to the acidic conditions present in the stomach it was important to identify whether the lead compounds might hydrolyse to their corresponding formamide compounds in-vivo. To determine this, Compound 41 was stirred for 6 hours in 1M acetic acid (pH 2.37), mimicking the length of time oral drugs are present in the stomach. The NMR data obtained after 6 hours was identical to that of compound 41 before the assay (FIG. 18). In addition to the NMR, IR analysis further confirmed the presence of the isocyanide group post treatment with acid, as shown by the sharp signal at approximately 2100 cm⁻¹.

### 7. Mode of Action Studies

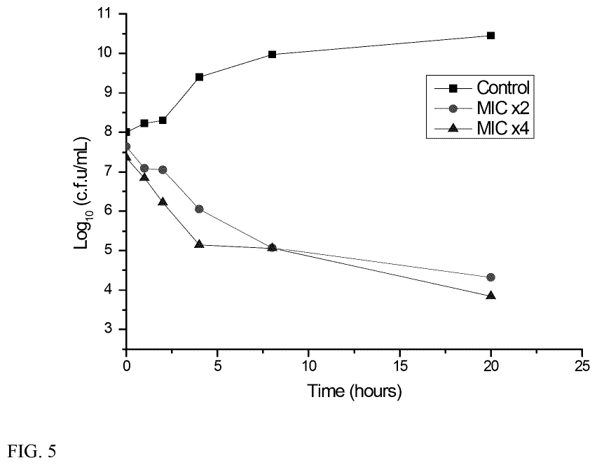

Investigations into the precise mode of action of these compounds was next studies, with time-kill assays, resistance studies, live-dead stain and morphological analysis of bacteria exposed to the compounds providing an insight into the specific mode of action. Time-kill assays were used to evaluate whether the compounds were bacteriostatic or bactericidal. Compound 41 showed bactericidal activity (defined as a minimal 3-log reduction in bacterial titre) against late-exponential phase MRSA 252 (re-suspended in TSB), with a 4-log reduction in bacterial density being measured over 20 hours exposure at concentrations of just 2×MIC (FIG. 5).⁽³¹⁾

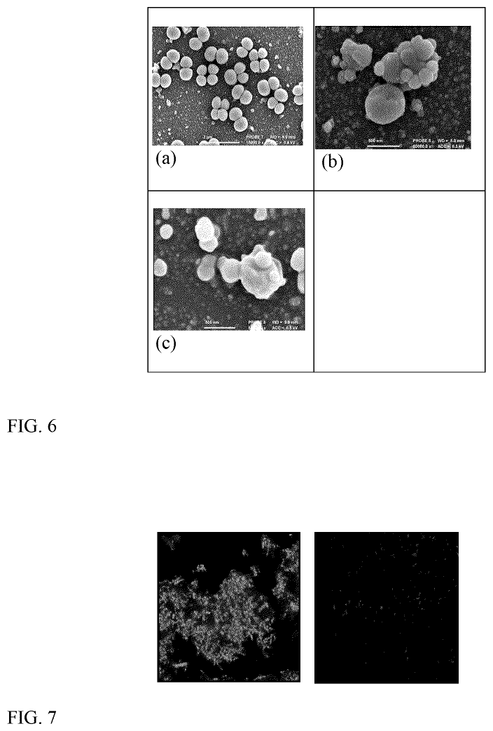

Scanning Electron Microscopy (SEM) is a powerful technique typically used in antimicrobial drug discovery to reveal the morphological features of bacterial cells when exposed to novel compounds. In this study, SEM images revealed characteristic S. aureus cells when no isocyanide compounds were added (FIG. 6). The cells are typical of healthy S. aureus. In contrast, on exposure of compound 41 (4×MIC), following overnight incubation of the bacteria, SEM images showed a high degree of distortion in the cell shape, with highly irregular membranes (FIG. 6). Addition of 41 resulted in bleb-like structures on the cell surface with an increased abundance of extracellular debris also present, further demonstrating the antibiotic activity of the compound 41. Bleb-like distortions in the cell membrane are characteristic for cell membrane targeting antibiotics, and hence we hypothesize that compound 41 and the other vinyl isocyanides targets] the cell membrane in Gram positive bacteria.

Interestingly, permeabalizing the outer membrane of E. coli strain DH5a with polymixin B nanopeptides saw a reduction in the MIC for compound 41 from 280 μg/mL to 16 μg/mL. This result suggests that the inability of this class of compound to inhibit Gram negative bacterial species at low concentrations, is likely to be a result of their failure to cross the additional outer membrane that these organisms possess. The assay also demonstrated that the probable target site of action for these compounds is also present in Gram negative species (i.e. the cell membrane).

To attempt to further confirm whether the membrane was indeed targeted by compound 41, a LIVE/DEAD assay was performed, in which MRSA 252 was inoculated with two dyes: SYTO 9 and propidium iodide (PI). Cells with compromised membranes will stain fluorescent red whilst those cells with intact membranes will stain fluorescent green. At a concentration of 4×MIC, when treated with compound 41, the few cells that remained on the surface, appeared red suggesting significant membrane damage, which supports the hypothesis that the cell membrane is involved in the mode of action of for compound 41. Contrary to this, cells with no antibiotic present stained fluorescent green (FIG. 7).

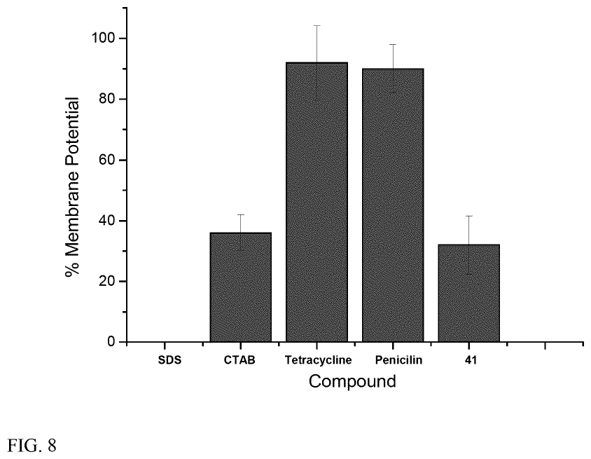

We next looked to further confirm the cell membrane as being the target site of action for our compounds, by undertaking a membrane depolarization assay using the voltage sensitive dye, 3,3′-dipropylthiacarbocyanine iodide (DiSC₃(5)). Compounds that target the bacterial cell membrane are often investigated (as above) using the LIVE/DEAD assay kit, however, these DNA binding dyes do not detect changes in the membrane potential. The membrane potential assay revealed that after just 180 minutes, 16 μg/mL of compound 41 was shown to cause a 70% reduction in the proton motive force of MRSA bacteria, with results comparable to the non-specific detergent cetyltrimethylammonium bromide (CTAB) (FIG. 8).

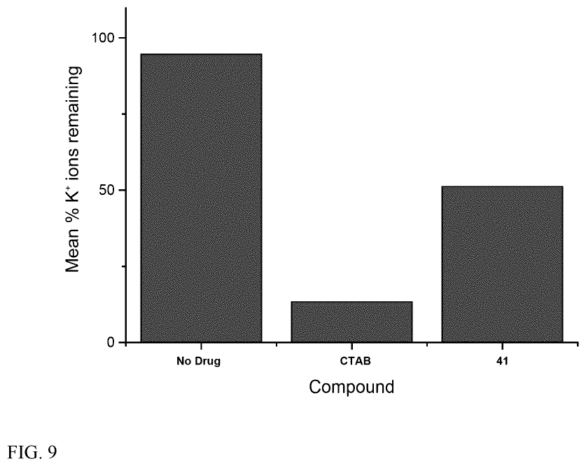

After demonstrating that compound 41 induced MRSA membrane depolarization, potassium levels in the cells were next measured to elucidate whether or not the physical integrity of the cell membrane was also affected. This particular assay revealed that exposure of MRSA cells to compound 41 for just 60 minutes, resulted in the dissipation of 50% of cellular K+ ions, further showing the rapid bactericidal properties of this novel class of compound (FIG. 9).

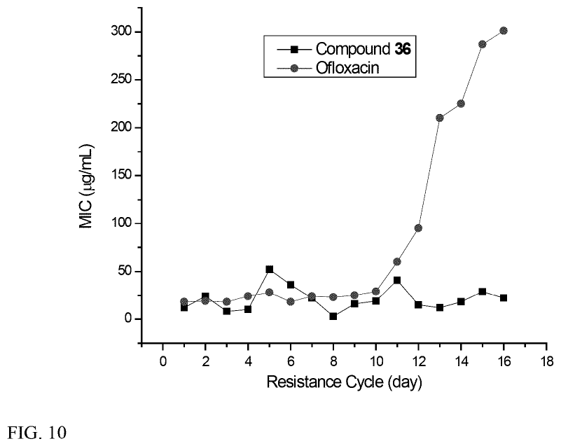

Next a serial passage study was undertaken in order to elucidate the speed at which target bacteria might either evolve resistance or select for persister cells within that population following exposure to compound 36. However, after 18 serial passage cycles, no increase in the MIC of 36 to MRSA 252 was observed (FIG. 10), in comparison with ofloxacin, which showed a greater than 10-fold increase in MIC over the same number of cycles.⁽³¹⁾ The absence of increase in the MIC over a serial passage experiment often suggests either a non-specific mode of action, but with corresponding eukaryotic toxicity. In this case the absence of cytotoxicity to eukaryotic cells and lack of hemolytic activity suggests that the novel vinyl isocyanide compounds that we have developed specifically target processes unique to prokaryotic cells.

### 8. Anti-Fungal Activity

The demand for new antibiotics is well-document, however, there is also an urgent need for the development of new antifungals to treat invasive fungal infections that are becoming increasingly resistant to our current arsenal of anti-fungals. Fungal infections specifically those caused by Candida, Cryptococcus and Aspergillus infect more than 1.5 million humans each year resulting in a mortality rate of over 50%.⁽³²⁾ The discovery of treatments for fungal infections is however intrinsically more difficult than that of bacterial infections, since fungi are also eukaryotic species, and hence they share many common biochemical and morphological features as mammalian cells.⁽³³⁾ The antifungal activity of a number of our vinyl isocyanide compounds was conducted by CO-ADD. Initial primary screening assays revealed the extremely potent antifungal activity for a number of our compounds. All seven compounds tested were shown to inhibit a number of fungal strains at concentrations (low M) with MIC values similar to those of currently used anti-fungal drugs (Table 2).

TABLE 2
Anti-fungal activity for the lead compounds 13, 14, 36, 38 and 41

| | MIC (μg/mL) | | | | | |
|---|---|---|---|---|---|---|
| | | C. neoformans | | | | |
| | C. albicans | H99; | C. glabrata | C. tropicalis | C. tropicalis | C. deuterogattii |
| | ATCC | ATCC | ATCC | ATCC | ATCC | ATCC |
| Compound | 90028 | 208821 | 90030 | 750 | 450 | 32609 |
| 13 | 4-8 | 4-8 | >32 | 16 | 4 | 4 |
| 14 | 32 | >32 | >32 | >32 | >32 | 32 |
| 15 | 8 | 8 | 32 | 16 | 4 | 4 |
| 22 | 16 | >32 | >32 | >32 | >32 | >32 |
| 36 | 16 | 4-8 | 32 | 32 | 2 | 2 |
| 38 | 2-4 | 4 | >32 | 8-16 | 2 | 2 |
| 41 | 2-4 | 2 | 32 | 32 | 1-2 | 1-2 |

### Methods

### Chemistry

All preparative details including general procedures and the synthesis of vinyl isocyanides, and their full characterization are detailed in the supporting information.

### Biology

Bacterial strains used in this study were recovered from Prof. Toby Jenkins and Dr. Maisem Laabei's collection of bacterial isolates.

### Bacterial Growth

E. coli DH5α, P. aeruginosa PA01 and the 53 S. aureus strains were recovered from frozen (−80° C.) glycerol (15% v/v) stocks on Lysogeny agar (LA)—Gram negative and trypticase soy agar (TSA)—plates at 37° C. for 24 hours. Single colonies were placed in either 3 mL Lysogeny broth (LB)—Gram negative or trypticase soy broth (TSB)—Gram positive and incubated at 37° C., 250 rpm for 18 hours.

### Disk Diffusion

The antibiotic activity was determined using a Kirby-Bauer method according to Clinical Standard Laboratory Institute (CSLI) Guidelines (2017). Briefly, 180 μL of a 1 in 200 dilution (in LB or TSB) of overnight cultures of selected Gram negative and Gram positive bacteria were applied to an agar plate containing the solid growth medium, Mueller-Hinton agar (MHA). Sterile discs, inoculated with 50 μL of antibiotic, were first added to the agar plate before the plates were incubated for 24 hours at 37° C. Following incubation, the zone of inhibition (if it existed) was recorded.

### Minimum Inhibition Concentration (MIC)

Antibiotic MIC's were determined by a broth micro-dilution method according to Clinical Standard Laboratory Institute (CSLI) Guidelines (2017). Briefly, 96-well microplates, each containing 195 μL of the 1:2 dilution antibiotic in TSB, were inoculated with 5 μL of overnight cultured bacteria, diluted to give a starting bacterial concentration of 5×10⁵ CFU/mL. The optical density of each well inoculated was recorded every 12 minutes over an 18 hour period at 37° C. The data from this was plotted in OriginPro8 (OriginLab) and sigmoidal curves fitted using the dose response function. Fitted values for each curve were used to calculate the MIC.

### Systemic Toxicity Assays

Galleria mellonella wax worms purchased from www.livefoods.co.uk were inoculated with 10 μL of a series of dilutions of the antibiotics synthesised in-house. The antibiotic concentrations chosen for this study were the following: 1000 μg/mL, 500 μg/mL, 250 μg/mL, 125 μg/mL and 31.25 μg/mL. Each dilution was injected into 10 individual wax worms through their last pro-leg. The injected wax worms were stored at 25° C. for 5 days. The cytotoxicity was determined as the percentage survival rate of Galleria mellonella Wax worms after 5 days inoculation.

Manduca sexta were first grown to their fifth instar stage of development before being inoculated behind one of the abdominal pro-legs with 10 μL of a set 1 mg/mL concentration of antibiotic. Each antibiotic was injected into 5 individual Manduca. The mass of each hornworm was measured before and up to 72 hours after injection at set 24 hour intervals. The systemic toxicity was determined as the percentage survival and mass growth relative to the positive control at the pre-determined set intervals.

### Biofilm Assays

Prevention: 100 μL of a mixture of TSB supplemented with 0.5% glucose and antibiotic (1:1) was added to individual wells in 96-well plate. The wells were then inoculated with 2.5 of an overnight culture of bacteria and incubated at 37° C. for 24 hours. Following the pre-determined length of time, the medium in each well was discarded, washed twice with PBS before 150 μL of a 1% crystal violet solution was added and the plate was left to incubate at room temperature for a further 30 minutes. Each well was then washed a further 4× with PBS before 200 μL 7% acetic acid was added. The absorbance of each well was then measured at OD₅₉₅.

Eradication: To develop biofilms, 2.5 μL of an overnight culture of bacteria was added to individual wells in a 96-well plate each containing 100 μL TSB supplemented with 0.5% glucose and incubated at 37° C. for 24 hours. Following this, the medium in each well was first discarded and washed once with PBS before set concentrations of antibiotic was added and incubated at 37° C. for a further 18 hours. The medium in each well was then discarded, washed 4× with PBS before 150 μL 1% crystal violet was added and the plate was left to incubate at room temperature for a further 30 minutes. Following incubation, the medium in each well was first discarded before being washed 4× with PBS. 200 μL 7% acetic acid was then added and the wells were measured at OD₅₉₅.

### Cytotoxicity Assay

HEK293 cells were counted manually in a Neubauer haemocytometer and then plated in the 384-well plates containing the compounds to give a density of 6000 cells/well in a final volume of 50 μL. DMEM supplemented with 10% FBS was used as growth media and the cells were incubated together with the compounds for 20 hours at 37° C. in 5% CO2.

Cytotoxicity (or cell viability) was measured by fluorescence, excitation: 560/10 nm, emission: 590/10 nm (F560/590), after addition of 5 μL of 25 μg/mL Resazurin (2.3 μg/mL final concentration) and after incubation for further 3 h at 37° C. in 5% CO2. The fluorescence intensity was measured using a Tecan M1000 Pro monochromator plate reader, using automatic gain calculation CC₅₀ (concentration at 50% cytotoxicity) were calculated by curve fitting the inhibition values vs. log(concentration) using sigmoidal dose-response function, with variable fitting values for bottom, top and slope. The curve fitting was implemented using Pipeline Pilot's dose-response component.

### Time-Kill Assay

The mode of inhibitory action of our novel class of compounds was determined by measuring the decrease in CFU over time. An overnight culture of MRSA 252 bacteria was diluted to 10⁷ before being centrifuged at 2000 rpm for 5 minutes and later washed with PBS. The pellets were re-suspended in TSB with the antibiotic added at 2× and 4×MIC and incubated at 37° C. Bacterial suspensions mixed with 1 M saline served as a control. Bacterial survivors were determined by plating serial dilutions on to TSA plates at 0, 1, 2, 4, 8 and 24 hours after incubation at 37° C.

### Cell Morphology Images

The cell morphology of MRSA 252 cells present on Melinex® films with or without antibiotic treatment was determined by Scanning Electron Microscope (SEM). Single colonies of MRSA 252 were added to individual wells containing Melinex® films and 3 mL TSB and incubated at 37° C. for 18 hours with minimal agitation (70 rpm). The growth media was then exposed to the antibiotics at various concentrations and incubated for a further 8 hours. Vancomycin treated wells served as a positive control whilst a well containing no antibiotic served as the negative control. Prior to observations, samples were fixed using 2.5% glutaraldehyde in 0.1 M sodium cacodylate buffer (SCB) (pH 7.3) for 90 minutes. The samples were then rinsed in 0.1 M SCB before 1% osmium tetroxide was added and left to incubate for 1 hour at room temperature. The samples were then washed twice with water and exposed to an acetone dehydration series of 50, 70, 90 and 2×100% acetone (v/v) acetone, followed by a chemical dehydration series of 100% acetone and hexamethyldisilazane (HMDS) at 50 and 2×100% HMDS (v/v) HMDS for 10 minutes at each concentration. After evaporation of HMDS for 2 hours, the samples were further dried overnight in a desiccator before being sputter-coated with a palladium-gold thin film. The samples were viewed with a Field Emission Scanning Electron Microscope (FESEM) (JEOL JSM6301F operating at 5 KV).

### LIVE/DEAD Assay

The LIVE/DEAD BacLight™ bacterial viability kit was purchased from Thermo Fischer Scientific and the assay was performed per manufactures instructions. The kit provides two nucleic acid stains; SYTO-9 and propidium iodide (PI), which allows live bacteria with intact membranes to be distinguished from bacteria with compromised membranes. Single colonies of MRSA 252 were added to individual wells containing Melinex® films and 3 mL TSB and incubated at 37° C. for 18 hours with minimal agitation (70 rpm). The growth media was then exposed to the antibiotics at various concentrations and incubated for 10 hours. The medium was then discarded in each well, washed with PBS before 200 μL of a solution containing both nucleic acid stains was added (50 μL of each component in 10 mL PBS) and left to incubate in the dark for 15 minutes at room temperature. Following this, the Melinex® films were extracted and gently washed with PBS before being observed under the confocal microscope at a magnification of 20×.

### DiSC₃(5) Assay

The membrane potential of S. aureus SH1000 cells re-suspended in HEPES and glucose buffer was determined using the method detailed by Winkel et al. following exposure of the cells to 4×MIC of compound 41 and the controls over 1 hour at 37° C. Cultures of SH1000 were grown to OD₆₀₀ of 0.2 before being incubated further with 0.1 M KCl and 2 μM DiSC₃(5) for 30 minutes at 37° C. The cells were then exposed to controls and compound 41 (4×MIC) for 1 hour at 37° C. Subsequently, the cells were centrifuged and 1 mL of supernatant mixed with 1 mL DMSO; the centrifuged pellet was lysed in DMSO for 10 minutes and added to equal volumes of HEPES and glucose buffer. Extracellular and intracellular fluorescence was measured on a LS 45 luminescence spectrometer (PerkinElmer) at an excitation and emission of 622 nm and 670 nm respectively. Consequently, the membrane potential was calculated using the Nernst equation and expressed as a percentage of the initial value.

$$Δϑ = - \frac{R T}{F} ln ( \frac{D i S C 3 ( 5 ) inside}{D i S C 3 ( 5 ) outside} )$$

where, Δν=membrane potential, R=gas constant and F=Faraday constant

### Potassium Leakage Detection

The potassium leakage of S. aureus SH1000 cells exposed to compounds was conducted as per previously published methods. Briefly, compounds were incubated with mid-exponential-phase S. aureus SH1000 cells in HEPES buffer (˜10⁸ CFU/ml) for 60 minutes. Cells were then removed by centrifugation, and the supernatant was assayed for K⁺ efflux by using a Perkin-Elmer 11001B atomic absorption instrument in flame emission mode (wavelength, 766.5 nm; slit, 0.7 nm high; air-acetylene flame). Prior to measurements, the instrument was calibrated using analytical grade potassium standards.

### Resistance Testing

For resistance development by serial passaging, 5 μL of overnight cultured MRSA 252 cells were added to individual wells in a 96-well plate containing 195 μL of TSB supplemented with a range of antibiotic concentrations and incubated at 37° C. for 18 hours. Ofloxacin served as a control. Wells where bacterial growth was visible after 18 hours were plated on TSA plates and incubated at 37° C. for a further 18 hours. Single colonies obtained from the overnight cultured bacteria were then inoculated into fresh TSB medium, incubated at 37° C. for 18 hours before being used in the next cycle of the resistance testing. This process continued for 18 cycles.

### Anti-Fungal Activity

Fungal strains were cultured for 3 days on Yeast Extract-Peptone Dextrose (YPD) agar at 30° C. A yeast suspension of 1×10⁶ to 5×10⁶ CFU/mL (as determined by OD₅₃₀) was prepared from five colonies. The suspension was subsequently diluted and added to each well of the compound-containing plates giving a final cell density of fungi suspension of 2.5×10³ CFU/mL and a total volume of 50 μL. All plates were covered and incubated at 35° C. for 36 hours without shaking.

Growth inhibition of C. albicans was determined measuring absorbance at 630 nm (OD₆₃₀), while the growth inhibition of C. neoformans was determined measuring the difference in absorbance between 600 and 570 nm (OD₆₀₀₋₅₇₀), after the addition of resazurin (0.001% final concentration) and incubation at 35° C. for 2 hours. The absorbance was measured using a Biotek Multiflo Synergy HTX plate reader. In both cases, the percentage of growth inhibition was calculated for each well, using the negative control (media only) and positive control (fungi without inhibitors) on the same plate. The MIC was determined as the lowest concentration at which the growth was fully inhibited, defined by an inhibition≥80% for C. albicans and an inhibition≥70% for C. neoformans. Due to a higher variance in growth and inhibition, a lower threshold was applied to the data for C. neoformans. In addition, the maximal percentage of growth inhibition is reported as DMax, indicating any compounds with marginal activity. Hits were classified by MIC≤16 μg/mL or MIC≤10 μM in either replicate (n=2 on different plates).

### Experimental Details

Preparation details (including all general procedures) for the vinyl isocyanide compounds and their precursor aldehydes if synthesized.

### Biological Data

Primary Screening disc diffusion results (Table 3)
% survival of Galleria mellonella (Table 4)
S. aureus disc diffusion results (Table 5)
Cytotoxicity data for lead complexes (Table 6)

### Stability Studies

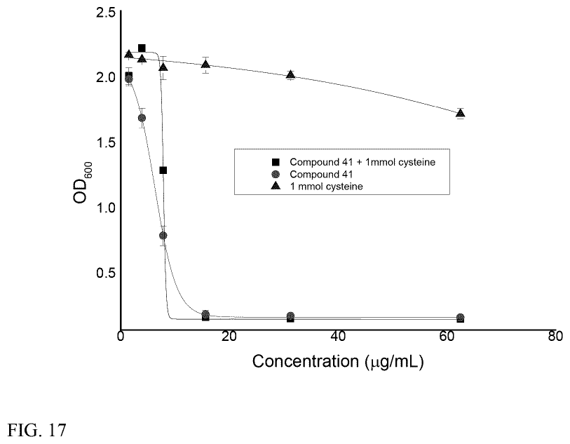

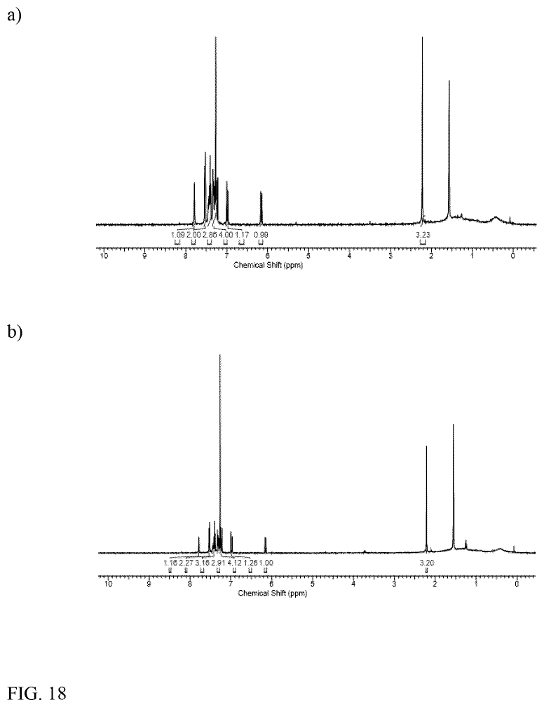

¹H NMR spectra of compound 41 after 6 months storage in organic solvent (FIG. 11)
¹H NMR spectra of compound 39 in the presence of glutathione at set intervals (FIGS. 12 to 16)
Plotted MIC curve for complex 41 against MRSA 252 before and after cysteine addition (FIG. 17)
¹H NMR spectra of complex 41 before and after exposure to 1 M acetic acid (FIG. 18)

### General Procedure 1: Horner-Wadsworth-Emmons Protocol

To a round bottom flask, the previously made phosphonate isocyanide (2 equivalents) was dissolved in 5 mL anhydrous THF, cooled to −78° C. and purged with N₂. LiHMDS (2.5 equiv.) was then added to the reaction vessel dropwise and left to stir for 20 minutes. Following this, an aldehyde source (1 equiv.) was dissolved in the minimum amount of TI-IF, added to the reaction mixture and left to stir overnight. The reaction was monitored by TLC. After completion of the reaction (i.e. no starting material was present in the TLC) the reaction solution was opened to the atmosphere, quenched with phosphate buffer, filtered after adding MgSO₄ and concentrated under reduced pressure. The crude mixture was then purified by silica gel chromatography to yield the desired compound.

### General Procedure 2: Heck Cross Coupling

Triethylamine (1.5 equiv.) and styrene (1.5 equiv.) were added to a solution of the aryl halide (1 equiv.), Pd(OAc)₂ (0.1 equiv.) and tri(o-tolyl)phosphine (0.2 equiv.) in DMF. The reaction was heated to 120° C. and refluxed overnight. The reaction was then cooled to 0° C. before a 1:1 mixture of ether and hexanes was added and stirred for an additional 30 minutes. The resulting precipitate was filtered using a plug of celite. The filtrate was collected, extracted with DCM, washed with H₂O and brine, dried over MgSO₄ and concentrated under vacuo. The desired product was purified using silica gel chromatography.

### General Procedure 3: Amide Formation

To the appropriate amine containing compound (1 equiv.) in DCM was added acetic anhydride (1.2 equiv.). The resulting solution was stirred overnight at room temperature. Following this, the reaction was diluted with DCM and washed with saturated Na₂CO₃. The organic layer was then extracted, dried with MgSO₄ and concentrated under vacuo to give the desired compound.

### General Procedure 4: Nitro Reduction/Ketal Removal

The appropriate nitro compound (1 equiv.) was first suspended in a 5:1 mixture of ethanol and H₂O before iron powder (4 equiv.) and 1 mL saturated ammonium chloride was added. The mixture was heated to 80° C. for 3 h before being cooled, filtered through celite and concentrated under vacuo. The resulting residue was partitioned between DCM and H₂O, with the organic layer dried with MgSO₄, filtered and concentrated under reduced pressure to give the title compound.

### Diethyl(isocyanomethyl)phosphonate (1a)

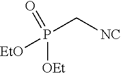

A solution of diethyl-N-(formyl)aminomethylphosphonate (8.18 g, 0.04 mol) in DCM was purged with N₂ and cooled to −78° C. before triethylamine (51.44 mL, 0.38 mol) and dropwise methane-sulfonyl chloride (7.70 mL, 0.10 mol) was added. After 16 hours, the resultant reaction mixture was quenched with aqueous NaHCO₃, washed with DCM, dried with MgSO₄ and concentrated under reduced pressure. The residual foul-smelling brown oil was purified by silica gel chromatography (ethyl acetate: petroleum ether (50:50)) affording a pale yellow oil. ¹H NMR (300 MHz, CDCl₃): δ_{H}=1.35 (t, J=7.3 Hz, 6H), 3.75 (d, J=1.0 Hz, 2H), 4.20 (q, J=7.0 Hz, 4H). ¹³C NMR (125 MHz, CDCl₃): δ_{c}=16.3, 37.5 (d, J=155.5 Hz), 63.9, 160.6. ³¹P NMR (125 MHz, CDCl₃): δₚ=14.2. IR (film, cm⁻¹): ν=2152.40 (N—C). R_{f} value: 0.34 (50% ethyl acetate: 50% petroleum ether)

### Diisopropyl (isocyanomethyl)phosphonate (1b)

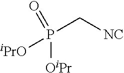

A solution of diisopropyl-N-(formyl)aminomethylphosphonate (6.50 g, 0.03 mol) DCM was purged with N₂ and cooled to −78° C. before trimethylamine (40.91 mL, 0.30 mol) and dropwise methane-sulfonyl chloride (7.70 mL, 0.10 mol) was added. After 16 hours, the resultant reaction mixture was quenched with aqueous NaHCO₃, washed with DCM, dried with MgSO₄ and concentrated under reduced pressure. The residual foul-smelling brown oil was purified by silica gel chromatography (ethyl acetate:pentane (50:50)) affording a pale yellow oil. ¹H NMR (300 MHz, CDCl₃): δ_{H}=1.35 (d, J=6.3 Hz, 12H), 3.70 (d, J=15.8 Hz, 2H), 4.78 (sept, J=7.0 Hz, 2H). ¹³C NMR (125 MHz, CDCl₃): δ_{c}=23.9, 38.3 (d, J=157.4 Hz), 73.0, 160.4. ³¹P NMR (125 MHz, CDCl₃): δₚ=13.0. IR (film, cm⁻¹): ν=2151.22 (N—C). R_{f} value: 0.32 (50% ethyl acetate: 50% hexane). HRMS (ESI) calculated for C₈H₁₆NO₃P [M+H]⁺: Theoretical m/z=228.0760 Measured m/z=228.0752

### 4-(2-Isocyanovinyl)phenol (2)

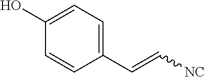

Following general procedure 1: Diisopropyl(isocyanomethyl)phosphonate (435 mg, 2.45 mmol), 4-hydroxybenzaldehyde (100 mg, 0.82 mmol) and LiHMDS (4.91 mL, 4.91 mmol) were stirred in anhydrous THE (7.5 mL) for 18 hours. The title compound was purified by silica column chromatography (15% ethyl acetate/hexane) to afford a pungent light brown crystalline solid in a 3.1 ratio of E- and Z-isomers (40 mg, 34%). Major isomer (E) ¹H NMR (300 MHz, CDCl₃): δ_{H}=5.15 (s, 1H), 6.15 (d, J=14.2 Hz, 1H) 6.80-6.95 (m, 2H), 6.95 (d, J=14.2 Hz, 1H), 7.25 (d, J=8.7 Hz, 2H). Minor isomer (Z)¹H NMR (300 MHz, CDCl₃): δ_{H}=5.75 (d, J=8.3 Hz, 1H), 6.85-6.95 (m, 3H), 7.65 (d, J=8.7 Hz, 2H). ¹³C NMR (75 MHz, CDCl₃): δ_{c}=109.3, 116.1, 116.4, 125.8, 128.8, 131.6, 131.9, 136.7, 157.5. IR (film, cm⁻¹): ν=3241.99 (O—H), 2925.49 (C—H), 2144.94 (N—C). R_{f} value: 0.55 (25% ethyl acetate: 75% petroleum ether). HRMS (ESI) calculated for C₉H₇NO [M−H]⁺: Theoretical m/z=144.0448 Measured m/z=144.0501

### (4-Hydroxyphenyl)acrylonitrile (3)

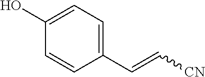 [SMILES: N#CC=Cc1ccc(O)cc1]

Following general procedure 1: Diethyl(cyanomethyl)phosphonate (435 mg, 2.45 mmol), 4-hydroxybenzaldehyde (100 mg, 0.82 mmol) and LiHMDS (3.28 mL, 3.28 mmol) were stirred in anhydrous THF (6 mL) for 18 hours. The title compound was purified by silica column chromatography (20% ethyl acetate/hexane) to afford a deep yellow pungent solid in a 6:1 ratio of E- and Z-isomers (31 mg, 21%). Major isomer (E) ¹H NMR (500 MHz, CD₃OD): δ_{H}=5.95 (d, J=16.6 Hz, 1H), 6.80 (d, J=8.8 Hz, 2H), 7.40 (d, J=16.6 Hz, 1H), 7.42 (d, J=8.8 Hz, 2H). Minor isomer (Z)¹H NMR (500 MHz, CD₃OD): δ_{H}=5.38 (d, J=12.2 Hz, 1H), 6.85 (d, J=8.8 Hz, 2H), 7.14 (d, J=12.2 Hz, 1H), 7.72 (d, J=8.8 Hz, 2H), 7.89 (br s, 1H). ¹³C NMR (125 MHz, CD₃OD): δ_{c}=93.0, 117.0, 120.3, 127.0, 130.7, 152.3, 162.0. IR (film, cm⁻¹): ν=3279.81 (O—H), 2220.42 (C—N). R_{f} value: 0.79 (20% ethyl acetate: 80% hexane). HRMS (ESI) calculated for C₉H₇NO [M−H]⁺: Theoretical m/z=144.0448 Measured m/z=144.0485

### 4-(2-Isocyanoethyl)phenol (4)

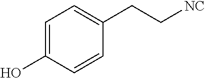

The title compound was prepared by first dehydrating N-(4-((tert-butyldimethylsilyl)oxy)phenethyl)formamide (0.98 g, 3.50 mmol), using MsCl (0.82 mL, 10.70 mmol) and Et₃N (4.48 mL, 32.20 mmol) in DCM (15 mL) to yield the crude silyl protected phenol-isocyanide as a brown oil that was then re-dissolved in ethanol (15 mL) and treated with excess KOH. After stirring for 2 hours, at room temperature, the crude reaction evaporation residue was partitioned between ethyl acetate and H₂O, the organics dried over MgSO₄ and then concentrated to give a pale brown oil that was purified by silica gel chromatography (ethyl acetate:pentane (50:50)) to yield a pale-yellow oil (0.42 g, 82%). ¹H NMR (300 MHz, (CD₃)₂SO): δ_{H}=2.74-2.81 (m, 2H), 3.62-3.70 (m, 2H), 6.72 (d, J=8.7 Hz, 2H), 7.07 (d, J=8.7 Hz, 2H), 9.33 (br s, 1H). ¹³C NMR (75 MHz, (CD₃)₂SO): δ_{c}=34.2, 43.3, 115.5, 127.8, 130.1, 155.9, 156.6. IR (film, cm⁻¹): ν=3030.65 (O—H), 2132.62 (N—C). R_{f} value: 0.55 (50% ethyl acetate: 50% petroleum ether)

### 4-Isocyanophenol (5)

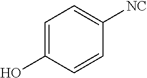

The title compound was prepared by first dehydrating N-(4-((tert-butyldimethylsilyl)oxy)phenyl)formamide (550 mg, 2.19 mmol) using MsCl (0.50 mL, 10.70 mmol) and Et₃N (2.68 mL, 19.71 mmol) in DCM (20 mL) to yield the crude O-silyl protected phenol-isocyanide as a brown oil that was then re-dissolved in ethanol (15 mL) and treated with excess KOH. After stirring for 2 hours, at room temperature, the crude reaction evaporation residue was partitioned between ethyl acetate and H₂O, the organics dried over MgSO₄ and then concentrated to give a pale brown oil that was purified by silica gel chromatography (pentane:ethyl acetate (80:20)) to yield a pale yellow oil (180 mg, 69%). ¹H NMR (300 MHz, CDCl₃): δ_{H}=6.85 (d, J=8.6 Hz, 2H), 7.26 (d, J=8.6 Hz, 2H). ¹³C NMR (75 MHz, CDCl₃): δ_{c}=116.3, 128.0, 157.1, 160.4. IR (film, cm⁻¹): ν=3382.31 (O—H), 2908.25 (C—H), 2947.60 (C—H), 2124.90 (N—C). R_{f} value: 0.45

### 2-Isocyanovinyl-benzene (6)

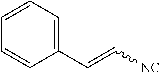

Following general procedure 1: Diethyl(isocyanomethyl)phosphonate (375 mg, 2.83 mmol), benzaldehyde (75 mg, 0.94 mmol) and LiHMDS (3.77 mL, 3.77 mmol) were stirred in anhydrous THF (6 mL) for 20 hours. The title compound was purified by silica column chromatography (10% ethyl acetate/hexane) to afford a pungent brown solid in a 2.5:1 ratio of E- and Z-isomers (46 mg, 38%). Major isomer (E) ¹H NMR (500 MHz, CDCl₃): δ_{H}=6.31 (d, J=14.2 Hz, 1H), 6.97 (d, J=14.7 Hz, 1H), 7.34-7.45 (m, 5H). Minor isomer (Z)¹H NMR (500 MHz, CDCl₃): δ_{H}=5.86 (d, J=9.3 Hz, 1H), 6.41 (d, J=9.3 Hz, 1H), 7.34-7.45 (m, 4H), 7.71 (d, J=8.8 Hz, 1H). ¹³C NMR (125 MHz, CDCl₃): δ_{c}=126.9, 129.0, 129.3, 129.6, 130.1, 137.0. IR (film, cm⁻¹): ν=3063.88 (C—H), 3028.52 (C—H), 2925.75 (C—H), 2121.38 (N—C). R_{f} value: 0.89 (10% ethyl acetate: 90% hexane)

### (E-1-Bromo-4-(2-isocyanovinyl)benzene (7)

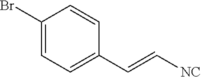

Following general procedure 1: Diisopropyl(isocyanomethyl)phosphonate (276 mg, 1.35 mmol), 4-bromobenzaldehyde (100 mg, 0.54 mmol) and LiHMDS (1.62 mL, 1.62 mmol) were stirred in anhydrous THF (6 mL) for 20 hours. The title compound was purified by silica column chromatography (5% ethyl acetate/hexanes) to afford a pungent, dark yellow solid as a single E-isomer (38 mg, 34%). ¹H NMR (500 MHz, CD₃CN): δ_{H}=6.55 (d, J=14.7 Hz, 1H), 7.05 (d, J=14.7 Hz, 1H), 7.37 (d, J=8.3 Hz, 2H), 7.58 (d, J=8.31 Hz, 2H). ¹³C NMR (125 MHz, CD₃CN): δ_{c}=116.2, 118.9, 129.2, 132.2, 134.0, 151.6. IR (film, cm⁻¹): ν=2923.56 (C—H), 2853.13 (C—H), 2120.13 (N—C). R_{f} value: 0.57 (5% ethyl acetate: 95% hexane)

### (E)-4-Methylphenyl vinyl isocyanide (8)

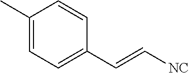

Following general procedure 1: Diisopropyl(isocyanomethyl)phosphonate (426 mg, 2.08 mmol), P-tolualdehyde (100 mg, 0.83 mmol) and LiHMDS (2.49 mL, 4.29 mmol) were stirred in anhydrous THF (6 mL) for 20 hours. The title compound was purified by silica column chromatography (30% ethyl acetate/hexanes) to afford a dark yellow solid as a single E-isomer (62 mg, 52%). ¹H NMR (500 MHz, CD₃CN): δ_{H}=2.34 (s, 3H), 6.47 (d, J=14.7 Hz, 1H), 7.05 (d, J=14.7 Hz, 1H), 7.22 (d, J=7.8 Hz, 2H), 7.35 (d, J=7.8 Hz, 2H). ¹³C NMR (125 MHz, CD₃CN): δ_{c}=21.4, 127.8, 130.7, 137.4, 137.5, 141.3. IR (film, cm⁻¹): ν=2924.42 (C—H), 2854.53 (C—H), 2164.4 (N—C). R_{f} value: 0.77 (30% ethyl acetate: 70% hexane)

### (2-Isocyanovinyl)-4-methoxybenzene (9)

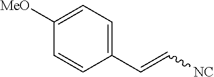

Following general procedure 1: Diethyl(isocyanomethyl)phosphonate (520 mg, 2.94 mmol), 4-methoxybenzaldehyde (100 mg, 0.73 mmol) and LiHMDS (4.41 mL, 4.41 mmol) were stirred in anhydrous THF (5 mL) for 18 hours. The title compound was purified by silica column chromatography (10% ethyl acetate/hexane) to afford a pungent dark red solid in a 2:1 ratio of E- and Z-isomers (41 mg, 35%). Major isomer (E) ¹H NMR (300 MHz, CDCl₃): δ_{H}=3.83 (s, 3H), 6.10 (d, J=14.5 Hz, 1H), 6.87 (d, J=8.9 Hz, 2H), 6.95 (d, J=13.6 Hz, 1H), 7.30 (d, J=9.8 Hz, 2H). Minor isomer (Z)¹H NMR (300 MHz, CDCl₃): δ_{H}=³0.85 (s, 3H), 5.65 (d, J=9.2 Hz, 1H), 6.87 (d, J=8.9 Hz, 2H), 7.30 (d, J=9.8 Hz, 1H), 7.70 (d, J=8.9 Hz, 2H). ¹³C NMR (125 MHz, CDCl₃): δ_{c}=55.6, 114.4, 114.7, 125.7, 128.4, 131.3, 131.7, 136.5, 160.8. IR (film, cm⁻¹): ν=2960.46 (C—H), 2118.44 (N—C). R_{f} value: 0.82 (10% ethyl acetate: 90% hexane)

### 3-(2-Isocyanovinyl)phenol (10)

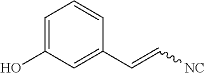

Following general procedure 1: Diethyl(isocyanomethyl)phosphonate (326 mg, 1.84 mmol), 3-hydroxybenzaldehyde (75 mg, 0.61 mmol) and LiHMDS (2.45 mL, 2.45 mmol) were stirred in anhydrous THF (7.5 mL) for 18 hours. The title compound was purified by silica column chromatography (30% ethyl acetate/hexane) to afford a pungent brown solid in a 5:2 ratio of E- and Z-isomers (40 mg, 46%). Major isomer (E) ¹H NMR (500 MHz, CDCl₃): δ_{H}=6.25 (d, J=14.7 Hz, 1H), 6.80-6.95 (m, 3H), 6.90 (d, J=14.2 Hz, 1H), 7.20-7.30 (m, 1H). Minor isomer (Z)¹H NMR (500 MHz, CDCl₃): δ_{H}=5.85 (d, J=9.3 Hz, 1H), 6.80-6.95 (m, 3H), 7.22 (d, J=8.8 Hz, 1H), 7.20-7.30 (m, 1H). ¹³C NMR (125 MHz, CDCl₃): 109.2, 115.6, 116.0, 128.5, 131.2, 131.5, 136.2, 157.0. IR (film, cm⁻¹): ν=3272.77 (O—H), 2924.35 (C—H), 2111.66 (N—C). R_{f} value: 0.63 (30% ethyl acetate: 70% hexane). HRMS (ESI) calculated for C₉H₇NO [M−H]⁺: Theoretical m/z=144.0448 Measured m/z=144.0471

### 2-(2-isocyanovinyl)phenol (11)

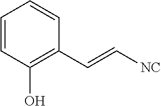

Following general procedure 1: Diisopropyl(isocyanomethyl)phosphonate (158 mg, 0.77 mmol), tert-butyl(2-(2-isocyanovinyl)phenoxy)dimethylsilane(100 mg, 0.38 mmol) and LiHMDS (0.85 mL, 0.85 mmol) were stirred in anhydrous THE (6 mL) for 18 hours. Post solvent extraction, ethanol and KOH were added to the compound and stirred for 3 hours to remove the tert-butyl dimethyl silane protecting group. Following this the title compound was purified by silica column chromatography (20% ethyl acetate/hexane) to afford a pungent brown solid as a single isomer (24 mg, 25%). ¹H NMR (300 MHz, (CD₃)₂CO): δ_{H}=6.66 (d, J=14.2 Hz, 1H), 6.77 (d, J=8.5 Hz, 1H), 6.85 (d, J=8.5 Hz, 1H), 6.99 (d, J=14.2 Hz, 1H), 7.04-7.27 (m, 2H), 7.58 (d, J=7.3 Hz, 1H). ¹³C NMR (75 MHz, (CD₃)₂CO): δ_{c}=79.6, 116.8, 117.2, 121.1, 130.5, 131.9, 134.4, 157.1. IR (film, cm⁻¹): ν=3361.04 (O—H), 2116.04 (N—C). R_{f} value: 0.20 (20% ethyl acetate: 80% hexane). HRMS (ESI) calculated for C₉H₈NO [M−H]⁺: Theoretical m/z=144.0448 Measured m/z=144.0471

### (E)-2-(2-Isocyanovinyl)-4-methoxyphenol (12)

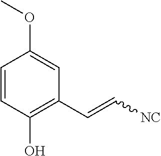

Following general procedure 1: Diethyl(isocyanomethyl)phosphonate (262 mg, 1.48 mmol), 2-hydroxy-5-methoxybenzaldehyde (75 mg, 0.49 mmol) and LiHMDS (1.97 mL, 1.97 mmol) were stirred in anhydrous THE (5 mL) for 18 hours. The title compound was purified by silica column chromatography (30% ethyl acetate/hexane) to afford a brown solid in a 5:1 ratio of E- and Z-isomers (38 mg, 44%). Major isomer (E) ¹H NMR (300 MHz, CDCl₃): δ_{H}=3.80 (s, 3H), 5.40 (br s, 1H), 6.60 (d, J=14.5 Hz, 1H), 6.70-6.80 (m, 3H), 7.05 (d, J=14.5 Hz, 1H). Minor isomer (Z)¹H NMR (300 MHz, CDCl₃): δ_{H}=3.80 (s, 3H), 5.40 (br s, 1H), 5.90 (d, J=9.5 Hz, 1H), 6.70-6.80 (m, 3H), 7.60 (d, J=9.5 Hz, 1H). ¹³C NMR (125 MHz, CDCl₃): δ_{c}=55.8, 112.8, 113.8, 116.7, 117.2, 120.7, 133.2, 148.4, 153.9, 164.1. IR (film, cm⁻¹): ν=3289.73 (O—H), 2835.44 (C—H), 2119.56 (N—C). R_{f} value: E=0.71, Z=0.91 (30% ethyl acetate: 70% hexane). HRMS (ESI) calculated for C₁₀H₉NO₂ [M+Na]⁺: Theoretical m/z=198.0525 Measured m/z=198.0532

### 5-Bromo-2-(2-isocyanovinyl)phenol (13)

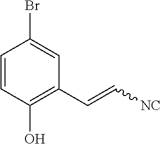

Following general procedure 1: Diethyl(isocyanomethyl)phosphonate (263 mg, 1.49 mmol), 5-bromosalicaldehyde (100 mg, 0.49 mmol) and LiHMDS (1.99 mL, 1.99 mmol) were stirred in anhydrous THF (6 mL) for 18 hours. The title compound was purified by silica column chromatography (15% ethyl acetate/hexane) to afford a yellow solid in a 4:1 ratio of E- and Z-isomers (15 mg, 28%). Major isomer (E) ¹H NMR (300 MHz, CDCl₃): δ_{H}=5.51 (br s, 1H), 6.57 (d, J=14.3 Hz, 1H), 6.69 (d, J=8.3 Hz, 1H), 7.00 (d, J=14.3 Hz, 1H), 7.30 (dd, J=2.5, 8.3 Hz, 1H), 7.38 (d, J=2.5 Hz). Minor isomer (Z)¹H NMR (300 MHz, CDCl₃): δ_{H}=5.87 (d, J=9.3 Hz, 1H), 6.72 (d, J=9.3 Hz, 1H), 6.86-6.95 (m, 2H), 7.35 (d, J=2.26 Hz, 1H). ¹³C NMR (125 MHz, CDCl₃): δ_{c}=112.9, 117.5, 131.2, 131.3, 132.8, 152.6. IR (film, cm⁻¹): ν=3198.11 (O—H), 2924.14 (C—H), 2853.3 (C—H), 2147.9 (N—C). R_{f} value: 0.55 (15% ethyl acetate: 85% hexane). HRMS (ESI) calculated for C₉H₆BrNO [M−H]⁺: Theoretical m/z=221.9560 Measured m/z=221.9552

### 3-bromo-4-(2-isocyanovinyl)phenol (14)

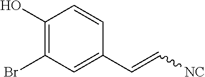

Following general procedure 1: Diisopropyl(isocyanomethyl)phosphonate (458 mg, 2.23 mmol), 3-bromo-4-hydroxybenzaldehyde (150 mg, 0.75 mmol) and LiHMDS (3.0 mL, 3.0 mmol) were stirred in anhydrous THE (7 mL) for 18 hours. The title compound was purified by silica column chromatography (20% ethyl acetate/hexane) to afford a deep yellow pungent solid in a 7:1 ratio of E- and Z-isomers (52 mg, 31%). Major isomer (E) ¹H NMR (500 MHz, (CD₃)₂CO): δ_{H}=6.65 (d, J=14.2 Hz, 1H), 7.05 (d, J=14.7 Hz, 1H), 7.06 (d, J=8.3 Hz, 1H), 7.40 (dd, J=2.0, 8.3 Hz, 1H), 7.72 (d, J=2.0 Hz, 1H), 9.40 (br s, 1H). Minor isomer (Z)¹H NMR (500 MHz, (CD₃)₂CO): δ_{H}=6.05 (d, J=9.3 Hz, 1H), 7.05 (d, J=9.3 Hz, 1H), 7.10(1H, d, J=8.3 Hz, 1H), 7.65 (dd, J=2.0, 8.8 Hz, 1H), 7.95 (d, J=3.0 Hz, 1H). ¹³C NMR (125 MHz, (CD₃)₂CO): δ_{c}=110.0, 116.6, 126.3, 127.6, 129.9, 131.6, 134.0, 135.0, 155.4. IR (film, cm⁻¹): ν=3078.76 (O—H), 2923.8 (C—H), 2151.03 (N—C). R_{f} value: 0.31 (20% ethyl acetate: 80% hexane). HRMS (ESI) calculated for C₉H₆BrNO [M−H]⁺: Theoretical m/z=221.9560 Measured m/z=221.9556

### 3-Bromo-4-(2-isocyanovinyl)phenol (15)

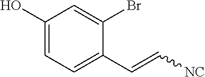

Following general procedure 1: Diethyl(isocyanomethyl)phosphonate (202 mg, 0.99 mmol), 2-bromo-4-hydroxybenzaldehyde (100 mg, 0.49 mmol) and LiHMDS (1.08 mL, 1.08 mmol) were stirred in anhydrous THF (6 mL) for 18 hours. The title compound was purified by silica column chromatography (50% ethyl acetate/hexane) to afford a yellow solid in a 5:1 ratio of E- and Z-isomers (30 mg, 56%). Major isomer (E) ¹H NMR (500 MHz, CD₃CN): δ_{H}=6.35 (d, J=14.2 Hz, 1H), 6.83 (dd, J=2.5, 7.8 Hz, 1H), 7.10 (d, J=2.5 Hz, 1H), 7.23 (d, J=14.7 Hz, 1H), 7.41 (d, J=8.8 Hz, 1H). Minor isomer (Z)¹H NMR (125 MHz, CD₃CN): δ_{H}=6.03 (d, J 9.3 Hz, 1H), 6.81 (d, J=9.3 Hz, 1H), 6.92 (dd, J=2.5, 8.8 Hz, 1H), 7.15 (d, J=2.5 Hz, 1H), 7.81 (d, J=8.8 Hz, 1H). ¹³C NMR (125 MHz, CD₃CN): δ_{c}=116.5, 117.1, 121.1, 125.6, 125.7, 129.6, 132.5, 135.9, 160.5. IR (film, cm⁻¹): ν=3185.19 (O—H), 2923.40 (C—H), 2853.06 (C—H), 2152.93 (N—C). R_{f} value: E=0.65, Z=0.77 (50% ethyl acetate: 50% hexanes). HRMS (ESI) calculated for C₉H₆BrNO [M−H]⁺: Theoretical m/z=221.9560 Measured m/z=221.9558

### 4-(2-Isocyanovinyl)benzene-1,2-diol (16)

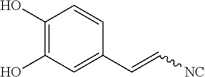

Following general procedure 1: Diisopropyl(isocyanomethyl)phosphonate (111 mg, 0.54 mmol), 3,4-bis-((tert-butyldimethylsilyl)oxy)benzaldehyde (100 mg, 0.27 mmol) and LiHMDS (0.66 mL, 0.66 mmol) were stirred in anhydrous THF (10 mL) for 18 hours and subsequently desilylated using ethoxide. The crude product was purified using silica gel chromatography (ethyl acetate 1:4 hexane) to afford the title compound as a pale-yellow oil (15 mg, 20%). ¹H NMR (300 MHz, CD₃CN): δ_{H}=6.25 (d, J=14.3 Hz, 2H), 6.60-6.70 (m, 2H), 6.73-6.81 (m, 2H). ¹³C NMR (75 MHz, CD₃CN): δ_{c}=114.5, 116.9, 121.4, 124.0, 125.50, 138.8, 147.2, 149.1. IR (film, cm⁻¹): ν=3220.54 (O—H), 2982.63 (C—H), 2933.75 (C—H), 2119.57 (N—C). R_{f} value: 0.05

### 2, 6-Dibromo-4-(2-isocyanovinyl)phenol (17)

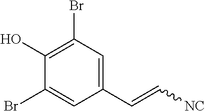

Following general procedure 1: Diethyl(isocyanomethyl)phosphonate (94 mg, 0.53 mmol), 3,5-dibromo-4-hydroxybenzaldehyde (75 mg, 0.26 mmol) and LiHMDS (0.54 mL, 0.54 mmol) were stirred in anhydrous THF (7 mL) for 18 hours. The title compound was purified by silica column chromatography (20% ethyl acetate/hexane) to afford a deep yellow pungent solid in a 4:1 ratio of E- and Z-isomers (27 mg, 30%). Major isomer (E) ¹H NMR (300 MHz, CD₃OD): δ_{H}=6.60 (d, J=14.5 Hz, 1H), 6.95 (d, J=14.5 Hz, 1H), 7.70 (s, 2H). Minor isomer (Z)¹H NMR (300 MHz, CD₃OD): δ_{H}=6.05 (d, J=9.2 Hz, 1H), 6.95 (d, J=9.5 Hz, 1H), 7.40 (s, 1H), 7.40 (d, J=3.5 Hz, 1H). ¹³C NMR (125 MHz, CD₃OD): δ_{c}=113.0, 117.2, 132.2, 133.7, 135.7, 154.1. IR (film, cm⁻¹): ν=3464.4 (O—H), 3071.2 (C—H), 2123.69 (N—C). R_{f} value: 0.70 (20% ethyl acetate: 80% hexane). HRMS (ESI) calculated for C₉H₅Br₂NO [M−H]⁺: Theoretical m/z=299.8665 Measured m/z=299.8652

### (E/Z)-2-(Isocyanovinyl)-1,4-dimethoxybenzene (18)

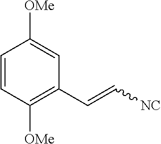

Following general procedure 1: Diisopropyl(isocyanomethyl)phosphonate (305 mg, 1.49 mmol), 2,5-dimethoxybenzaldehyde (100 mg, 0.59 mmol) and LiHMDS (1.79 mL, 1.79 mmol) were stirred in anhydrous THF (6 mL) for 20 hours. The title compound was purified by silica column chromatography (10% ethyl acetate/hexanes) to afford a light brown solid in a 9:1 ratio of F- and Z-isomers (39 mg, 35%). Major isomer (E) ¹H NMR (500 MHz, CDCl₃): δ_{H}=3.76 (s, 3H), 3.84 (s, 3H), 6.50 (d, J=14.7 Hz, 1H), 6.80-6.90 (m, 3H), 7.05 (d, J=14.2 Hz, 1H). Minor isomer (Z)¹H NMR (500 MHz, CDCl₃): δ_{H}=3.76 (s, 3H), 3.84 (s, 3H), 5.85 (d, J=9.3 Hz, 1H), 6.80-6.90 (m, 3H), 6.91 (d, J=9.3 Hz, 1H). ¹³C NMR (125 MHz, CDCl₃): δ_{c}=55.6, 55.8, 111.9, 113.8, 115.6, 121.2, 132.7, 151.8, 153.2. IR (film, cm⁻¹): ν=2944.15 (C—H), 2835.15 (C—H), 2118.40 (N—C). R_{f} value: 0.56 (10% ethyl acetate: 90% hexane)

### (E-1-Bromo-2-(2-isocyanovinyl)-4-methoxybenzene (19)

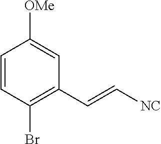

Following general procedure 1: Diisopropyl(isocyanomethyl)phosphonate (237 mg, 1.16 mmol), 2-bromo,5-methoxybenzaldehyde (100 mg, 0.46 mmol) and LiHMDS (1.40 mL, 1.40 mmol) were stirred in anhydrous THF (7 mL) for 18 hours. The title compound was purified by silica column chromatography (20% ethyl acetate/hexanes) to afford a pungent yellow solid as a single F-isomer (51 mg, 47%). ¹H NMR (500 MHz, CD₃CN): δ_{H}=3.82 (s, 3H), 6.55 (d, J=14.7 Hz, 1H), 6.90 (dd, J=3.0, 8.8 Hz, 1H), 7.12 (d, J=3.0 Hz, 1H), 7.28 (d, J=14.2 Hz, 1H), 7.55 (d, J=8.8 Hz, 1H). ¹³C NMR (125 MHz, CD₃CN): δ_{c}=55.5, 113.6, 115.0, 118.7, 135.1, 136.1, 160.5. IR (film, cm⁻¹): ν=2924.48 (C—H), 2852.07 (C—H), 2121.60 (N—C). R_{f} value: 0.45 (20% ethyl acetate: 80% hexane)

### (E)-2,4-Dichloro-1-(2-isocyanovinyl)benzene (20)

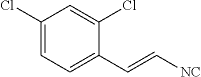

Following general procedure 1: Diisopropyl(isocyanomethyl)phosphonate (293 mg, 1.43 mmol), 2,4-dichlorobenzaldehyde (100 mg, 0.57 mmol) and LiHMDS (1.72 mL, 1.72 mmol) were stirred in anhydrous THF (6 mL) for 20 hours. The title compound was purified by silica column chromatography (10% ethyl acetate/hexanes) to afford a pungent, dark yellow solid as a single F-isomer (55 mg, 49%). ¹H NMR (500 MHz, CD₃Cl): δ_{H}=6.30 (d, J=14.2 Hz, 1H), 7.25-7.27 (m, 1H), 7.28 (d, J=14.7 Hz, 1H), 7.36 (d, J=8.3 Hz, 1H), 7.46 (d, J=2.0 Hz, 1H). ¹³C NMR (125 MHz, CD₃Cl): δ_{c}=127.4, 127.7, 130.1, 132.2, 136.2. IR (film, cm⁻¹): ν=2923.98 (C—H), 2852.71 (C—H), 2124.87 (N—C). R_{f} value: 0.89 (10% ethyl acetate: 90% hexane)

### (E)-4-(2-Isocyanovinyl)-N,N-dimethylalanine (21)

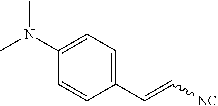

Following general procedure 1: Diisopropyl(isocyanomethyl)phosphonate (275 mg, 1.34 mmol), 4-dimethylaminobenzaldehyde (100 mg, 0.67 mmol) and LiHMDS (2.01 mL, 2.01 mmol) were stirred in anhydrous THF (7 mL) for 20 hours. The title compound was purified by silica column chromatography (20% ethyl acetate/hexanes) to afford a dark yellow solid as a mixture of E- and Z-isomers in a 5:1 ratio (51 mg, 44%). Major isomer (E) ¹H NMR (500 MHz, CDCl3): δ_{H}=3.01 (s, 3H), 6.10 (d, J=14.2 Hz, 1H), 6.65 (d, J=8.8 Hz, 2H), 6.85 (d, J=14.7 Hz, 1H), 7.20 (d, J=8.8 Hz, 2H). Minor isomer (Z)¹H NMR (500 MHz, CDCl₃): δ_{H}=3.03 (s, 3H), 5.60 (d, J=9.3 Hz, 1H), 6.70 (d, J=9.3 Hz, 1H), 7.40 (d, J=8.3 Hz, 2H), 7.65 (d, J=8.3 Hz, 2H). ¹³C NMR (125 MHz, CDCl₃): δ_{c}=40.4, 111.9, 112.2, 128.2, 130.0, 131.1, 132.2, 137.1, 151.6, 163.6. IR (film, cm⁻¹): ν=2908.28 (C—H), 2818.73 (C—H), 2114.22 (N—C). R_{f} value: 0.85 (20% ethyl acetate: 80% hexane)

### (2-Isocyanovinyl)-4-acetaamido benzene (22)

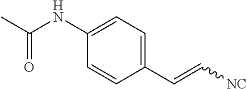

Following general procedure 1: Diethyl(isocyanomethyl)phosphonate (81 mg, 0.46 mmol), 4-acetamidobenzaldehyde (75 mg, 0.46 mmol) and LiHMDS (1.84 mL, 184 mmol) were stirred in anhydrous THF (5 mL) for 18 hours. The title compound was purified by silica column chromatography (15% ethyl acetate/hexane) to afford a brown pungent solid in an 8:1 ratio of E- and Z-isomers (27 mg, 32%). Major isomer (E) ¹H NMR (300 MHz, CDCl₃): δ_{H}=2.20 (s, 3H), 6.24 (d, J=14.3 Hz, 1H), 6.90 (d, J=14.3 Hz, 1H), 7.46 (br s, 1H), 7.30 (d, J=8.3 Hz, 2H), 7.54 (d, J=8.7 Hz, 2H). Minor isomer (Z)¹H NMR (300 MHz, CDCl₃): δ_{H}=2.23 (s, 3H), 5.80 (d, J=9.4 Hz, 1H), 7.30 (d, J=9.4 Hz, 1H), 7.60 (br s, 1H), 7.67-7.72 (m, 2H), 7.84 (d, J=8.3 Hz, 2H). ¹³C NMR (125 MHz, CDCl₃): δ_{c}=24.3, 109.6, 119.2, 119.6, 127.2, 129.6, 131.8 135.6, 139.2, 164.6, 168.3. IR (film, cm⁻¹): ν=3253.59 (N—H), 3071.34 (C—H), 2116.37 (N—C). R_{f} value: E=0.44, Z=0.48 (15% ethyl acetate: 85% hexane)

### Tert-butyl(4-(isocyanovinyl)phenyl)carbamate (23)

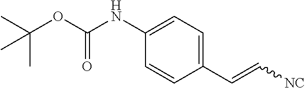

Following general procedure 1: Diisopropyl(isocyanomethyl)phosphonate (200 mg, 0.98 mmol), tert-butyl-4-formylphenylcarbamate (100 mg, 0.49 mmol) and LiHMDS (1.23 mL, 1.23 mmol) were stirred in anhydrous THE (6 mL) for 18 hours. The title compound was purified by silica column chromatography (hexane:ethyl acetate (95:5)) to the title compound as a brown oil as a mixture of E- and Z-isomers (4:1) (38 mg, 31%). Major isomer (E) ¹H NMR (300 MHz, (CD₃)₂CO): δ_{H}=1.45 (s, 9H), 6.35 (d, J=14.3 Hz, 1H), 6.87 (d, J=14.3 Hz, 1H), 7.30 (d, J=8.7 Hz, 2H), 7.50 (d, J=8.7 Hz, 2H), 8.24 (s, 1H). Minor isomer (Z)¹H NMR (300 MHz, (CD₃)₂CO): δ_{H}=1.45 (s, 9H), 5.82 (d, J=9.4 Hz, 1H), 7.24 (d, J=9.4 Hz, 1H), 7.43 (d, J=10.7 Hz, 2H), 7.50 (d, J=12.8 Hz, 2H). ¹³C NMR (75 MHz, (CD₃)₂CO): δ_{c}=28.3, 80.2, 118.7, 127.9, 136.6, 141.4, 153.1. IR (film, cm⁻¹): ν=2976.09 (C—H), 2857.55 (C—H), 2123.08 (N—C). R_{f} value: 0.30

### N-(4-(1-Isocyanoprop-1-en-2-yl)phenyl)methanesulfonamie (24)

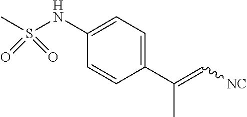

Following general procedure 1: Diisopropyl(isocyanomethyl)phosphonate (191 mg, 1.12 mmol), N-(4-acetylphenyl)methanesulfonamide (100 mg, 0.47 mmol) and LiHMDS (1.40 mL, 1.40 mmol) were stirred in anhydrous THF (5 mL) for 20 hours. The title compound was purified by silica column chromatography (ethyl acetate/hexane (1:3)) to afford a red pungent solid in a 6:1 ratio of E- and Z-isomers (40 mg, 37%). Major isomer (E) ¹H NMR (500 MHz, CDCl₃): δ_{H}=2.24 (d, J=1.5 Hz, 3H), 3.06 (s, 3H), 6.03 (s, 1H), 6.48 (s, 1H), 7.22 (d, J=8.8 Hz, 2H), 7.33 (d, J=8.8 Hz, 2H). Minor isomer (Z)¹H NMR (500 MHz, CDCl₃): δ_{H}=2.10 (d, J 1.5 Hz, 3H), 3.08 (s, 3H), 5.84 (s, 1H), 7.06 (s, 1H), 7.24 (d, J=8.8 Hz, 2H), 7.50 (d, J=8.8 Hz, 2H). ¹³C NMR (125 MHz, CDCl₃): δ_{c}=16.9, 39.8, 119.8, 120.2, 127.3, 127.6, 127.8, 128.0, 129.1, 137.6, 142.8. IR (film, cm⁻¹): ν=3249.99 (N—H) 3024.61 (N—H), 2983.74 (C—H), 2930.51 (C—H), 2114.18 (N—C). R_{f} values: 0.12 (25% ethyl acetate: 75% hexane). HRMS (ESI) calculated for C₁₁H₁₂N₂O₂S [M+Na]⁺: Theoretical m/z=259.0512 Measured m/z=259.0513

### 3-(2-Isocyanovinyl)-1H-indole (25 and 26)

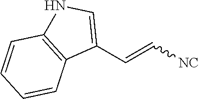

Following general procedure 1: Diethyl(isocyanomethyl)phosphonate (487 mg, 2.75 mmol), indole-3-carboxaldehyde (100 mg, 0.69 mmol) and LiHMDS (4.13 mL, 4.13 mmol) were stirred in anhydrous THF (6 mL) for 18 hours. The title compound was purified by silica gel column chromatography (50% ethyl acetate/hexane) to afford a mixture of E- and Z-isomers in a 3:1 ratio. The E-isomer was isolated as dark yellow solid (20 mg, 23%) with the Z-isomer isolated as a dark red solid (35 mg, 40%). Major isomer (E) ¹H NMR (500 MHz, CDCl₃): δ_{H}=6.35 (d, J=14.2 Hz, 1H), 7.14 (d, J=14.2 Hz, 1H), 7.25-7.35 (m, 2H), 7.36 (s, 1H), 7.43 (d, J=7.8 Hz, 1H), 7.70 (d, J=7.3 Hz, 1H), 8.35 (br s, 1H). Minor isomer (Z)¹H NMR (500 MHz, CDCl₃): δ_{H}=5.75 (d, J=8.8 Hz, 1H), 7.20-7.27 (m, 2H), 7.28 (d, J=8.3 Hz, 1H), 7.45 (d, J=8.3 Hz, 1H), 7.69 (d, J=8.3 Hz, 1H), 8.16 (d, J=2.5 Hz, 1H), 8.55 (br s, 1H). ¹³C NMR (125 MHz, CDCl₃): δ_{c}=107.1, 111.1, 119.9, 121.4, 123.4, 126.3, 130.2, 136.9, 163.1. IR (film, cm⁻¹): ν=3288.17 (N—H), 2926.27 (C—H), 2116.18 (N—C). R_{f} values: E=0.69, Z=0.78 (50% ethyl acetate: 50% hexane)

### N-Methyl-indole-3-carboxaldehyde

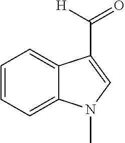

Indole-3-carboxaldehyde (1.00 g, 6.90 mmol) was treated with NaH (0.30 g, 8.30 mmol) in anhydrous THE (30 mL) at 0° C. for 10 minutes. Iodomethane (0.5 mL, 8.20 mmol) was then added to the resulting mixture and stirred for 5 hours. The reaction was then quenched with H₂O and extracted with ethyl acetate. The organic layer was then washed with H₂O and brine before being dried with MgSO₄ and concentrated in vacuo. The remaining residue was purified by silica column chromatography (50% ethyl acetate: 50% hexane) to yield a pale creamy solid (0.70 g, 67%). ¹H NMR (500 MHz, CDCl₃): δ_{H}=3.89 (s, 1H), 7.33-7.38 (m, 3H), 7.70 (s, 1H), 8.32 (d, J=7.3 Hz, 1H), 10.01 (s, 1H). ¹³C NMR (125 MHz, CDCl₃): δ_{c}=33.7, 109.7, 122.0, 122.9, 124.0, 139.1, 184.4. IR (film, cm⁻¹): ν=1651.55 (C═O). R_{f} value: 0.50 (50% ethyl acetate: 50% hexane). HRMS (ESI) calculated for C₁₀H₉NO [M+H]⁺: Theoretical m/z=160.0762 Measured m/z=160.0760. Melting point: 72° C.

### (E)-3-(2-Isocyanovinyl)-1-methyl-indole (27)

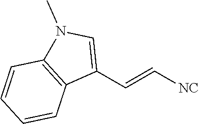

Following general procedure 1: Diethyl(isocyanomethyl)phosphonate (338 mg, 1.91 mmol), N-methyl-indole-3-carboxaldehyde (100 mg, 0.63 mmol) and LiHMDS (2.55 mL, 2.55 mmol) were stirred in anhydrous THF (6.0 ml) for 18 hours. The title compound was purified by silica column chromatography (50% ethyl acetate/hexane) to afford a deep red solid as a single E-isomer (32 mg, 28%). ¹H NMR (300 MHz, CDCl₃): δ_{H}=3.81 (s, 3H), 6.31 (d, J=14.3 Hz, 1H), 7.10 (d, J=14.3 Hz, 1H), 7.20 (s, 1H), 7.22-7.38 (m, 4H), 7.67 (d, J=7.5 Hz, 1H). ¹³C NMR (125 MHz, CDCl₃): δ_{c}=33.0, 106.2, 109.4, 110.0, 119.9, 121.1, 122.9, 125.2, 130.0, 130.7, 137.1, 162.9. IR (film, cm⁻¹): ν=3051.80 (C—H), 2929.57 (C—H), 2116.00 (N—C). R_{f} value: 0.70 (50% ethyl acetate: 50% hexane)

### N-Ethyl-indole-3-carboxaldehyde

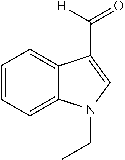

Indole-3-carboxaldehyde (1.00 g, 6.9 mmol) was treated with NaH (0.33 g, 8.3 mmol) in anhydrous THE (30 ml) at 0° C. for 10 minutes. Bromoethane (0.62 ml, 8.3 mmol) was then added to the resulting mixture and stirred for 5 hours. Following this, the reaction was quenched with H₂O and extracted with ethyl acetate. The organic layers were then washed with H₂O and brine before being dried with MgSO₄ and concentrated in vacuo. The remaining residue was purified by silica column chromatography (50% ethyl acetate/hexane) to yield a cream coloured solid (0.76 g, 64%). ¹H NMR (300 MHz, CDCl₃): δ_{H}=1.57 (t, J=7.3 Hz, 3H), 4.25 (q, J=7.3 Hz, 2H) 7.31-7.42 (m, 3H), 7.77 (s, 1H), 8.32 (d, J=6.9 Hz, 1H), 10.02 (s, 1H). ¹³C NMR (500 MHz, CDCl3): δ_{c}=15.0, 42.0, 109.9, 118.2, 122.2, 122.9, 123.9, 137.0, 137.4, 184.4. IR (film, cm⁻¹): ν=1651.55 (C═O). R_{f} value: 0.71(50% ethyl acetate: 50% hexane). HRMS (ESI) calculated for C₁₁H₁₁NO [M+H]⁺: Theoretical m/z=174.0918 Measured m/z=174.0918. Melting point=105° C.

### (2-Isocyanovinyl)-1-ethyl-indole (28)

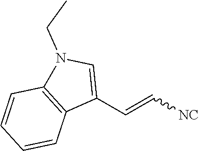

Following general procedure 1: Diethyl(isocyanomethyl)phosphonate (325 mg, 1.84 mmol), N-ethyl-indole-3-carboxaldehyde (100 mg, 0.613 mmol) and LiHMDS (2.43 mL, 2.43 mmol) were stirred in anhydrous THF (7 mL) for 20 hours. The title compound was purified by silica column chromatography (20% ethyl acetate/hexane) to afford a pungent light brown solid in a 9:1 ratio of E- and Z-isomers (36 mg, 31%). Major isomer (E) ¹H NMR (300 MHz, CDCl₃): δ_{H}=1.50 (t, J=7.5 Hz, 3H), 4.18 (q, J=7.5 Hz, 2H), 6.25 (d, J=14.3 Hz, 1H), 7.05 (d, J=14.3 Hz, 1H), 7.22-7.25 (m, 1H), 7.30 (td, J=1.0, 6.9 Hz, 1H), 7.36 (dt, J=1.0, 8.3 Hz, 1H), 7.68 (d, J=7.5 Hz, 1H). Minor isomer (Z)¹H NMR (500 MHz, CDCl₃): δ_{H}=1.53 (t, J=7.3 Hz, 3H), 4.26 (q, J=7.4 Hz, 2H), 5.70 (d, J=9.3 Hz, 1H), 7.24 (d, J=9.3 Hz, 1H), 7.27-7.32 (m, 2H), 7.40 (d, J=7.3 Hz, 1H), 7.67 (d, J=7.8 Hz, 1H), 8.06 (s, 1H). ¹³C NMR (125 MHz, CDCl₃): δ_{c}=15.2, 29.7, 110.1, 120.1, 122.9, 129.0, 130.4. IR (film, cm⁻¹): ν=2925.26 (C—H), 2115.43 (N—C). R_{f} values: E=0.72, Z=0.90 (20% ethyl acetate: 80% hexane)

### N-Isopropyl-indole-3-carboxaldehyde

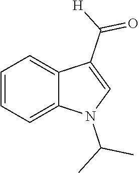

Indole-3-carboxaldehyde (1.00 g, 6.8 mmol) was treated with NaH (0.6 g, 13.7 mmol) in anhydrous THF (30 mL) at 0° C. for 10 minutes. Isopropyl iodide (1.4 mL, 13.7 mmol) was then added to the resulting mixture and stirred for 5 h. The reaction was then quenched with H₂O and extracted with ethyl acetate. The organic layer was then washed with H₂O and brine before being dried with MgSO₄ and concentrated in vacuo. The remaining residue was purified by silica column chromatography (50% ethyl acetate: 50% hexane) to yield a pale yellow solid (0.68 g, 54%). ¹H NMR (500 MHz, CDCl₃): δ_{H}=1.62 (d, J=6.9 Hz, 6H), 4.75 (sept, J=6.9 Hz, 1H), 7.31-7.37 (m, 2H), 7.43 (d, J=7.3 Hz, 1H), 7.86 (s, 1H), 8.32 (d, J=6.9 Hz, 1H), 10.03 (s, 1H). ¹³C NMR (125 MHz, CDCl₃): δ_{c}=22.5, 48.2, 110.2, 119.9, 122.1, 122.8, 123.7, 184.5. IR (film, cm⁻¹): ν=1642.76 (C═O). R_{f} value: 0.56 (50% ethyl acetate: 50% hexane). HRMS (ESI) calculated for C₁₂H₁₃NO [M+H]⁺: Theoretical m/z=188.1075 Measured m/z=188.1083. Melting point: 98° C.

### (E-3-(2-Isocyanovinyl)-1-isopropyl-indole (29)

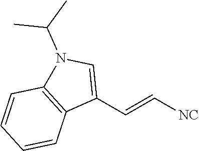

Following general procedure 1: Diethyl(isocyanomethyl)phosphonate (299 mg, 1.69 mmol), N-isopropyl-indole-3-carboxaldehyde (100 mg, 0.56 mmol) and LiI-HMDS (2.25 mL, 2.25 mmol) were stirred in anhydrous THF (7 mL) for 20 hours. The title compound was purified by silica column chromatography (20% ethyl acetate/hexane) to afford a dark yellow solid as a single E-isomer (29 mg, 26%). ¹H NMR (300 MHz, CDCl₃): δ_{H}=1.55 (d, J=6.8 Hz, 6H), 4.68 (sept, J=6.8 Hz, 1H), 6.33 (d, J=14.3 Hz, 1H), 7.13 (d, J=14.3 Hz, 1H), 7.21-7.33 (m, 2H), 7.36 (s, 1H), 7.42 (d, J=7.9 Hz, 1H), 7.67 (d, J=7.91 Hz, 1H). ¹³C NMR (125 MHz, CDCl₃): δ_{c}=22.6, 47.4, 106.0, 110.3, 120.1, 121.1, 122.6, 125.5, 125.9, 130.3. IR (film, cm⁻¹): ν=2925.21 (C—H), 2854.40 (C—H), 2116.07 (N—C). R_{f} value: 0.72 (20% ethyl acetate: 80% hexane)

### 4-Bromo-3-(2-isocyanovinyl)-1H-indole (30 and 31)

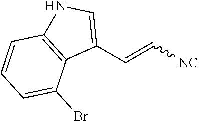

Following general procedure 1: Diethyl(isocyanomethyl)phosphonate (177 mg, 1.00 mmol), 4-bromo indole-3-carboxaldehyde (75 mg, 0.33 mmol) and LiHMDS (1.33 mL, 1.33 mmol) were stirred in anhydrous THF (6 mL) for 20 hours. The title compound was purified by silica column chromatography (50% ethyl acetate/hexane) to afford a pungent light brown solid in a 3:1 ratio of E- and Z-isomers (28 mg, 35%). Major isomer (E) ¹H NMR (300 MHz, CDCl₃): δ_{H}=6.07 (d, J=14.3 Hz, 1H), 7.09 (t, J=7.6 Hz, 1H), 7.34 (d, J=3.0 Hz, 1H), 7.38 (m, 1H), 7.43 (d, J=2.6 Hz, 1H), 7.97 (d, J=14.3 Hz, 1H), 8.45 (br s, 1H). Minor isomer (Z)¹H NMR (300 MHz, CDCl₃): δ_{H}=5.75 (d, J=9.2 Hz, 1H), 6.80 (d, J=9.2 Hz, 1H), 7.22-7.25 (m, 1H), 7.27-7.31 (m, 1H), 7.44-7.46 (m, 1H), 7.69 (d, J=8.4 Hz, 1H), 8.15 (d, J=2.9 Hz, 1H), 8.58 (br s, 1H). ¹³C NMR (125 MHz, CDCl₃): δ_{c}=110.2, 114.5, 118.0, 121.0, 123.2, 124.1, 126.4, 126.8, 128.5, 132.0. IR (film, cm⁻¹): ν=3658.20 (N—H), 2979.39 (C—H), 2888.24 (C—H), 2139.04 (N—C). R_{f} values: E=0.57, Z=0.80 (50% ethyl acetate: 50% hexane)

### (E/Z)-2-Bromo-3-(2-isocyanovinyl)naphthalene (32)

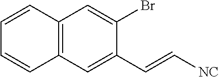

Following general procedure 1: Diisopropyl(isocyanomethyl)phosphonate (217 mg, 1.06 mmol), 1-bromo-2-napthaldehyde (100 mg, 0.43 mmol) and LiHMDS (1.27 mL, 1.27 mmol) were stirred in anhydrous THF (7 mL) for 18 hours. The title compound was purified by silica column chromatography (20% ethyl acetate/hexanes) to afford a light brown solid as a mixture of E- and Z-isomers in a 2:1 ratio (49 mg, 45%). Major isomer (E) ¹H NMR (500 MHz, CD₃CN): δ_{H}=6.65 (d, J=14.7 Hz, 1H), 7.62-7.74 (m, 4H), 7.91-8.01 (m, 2H), 8.36 (d, J=9.3 Hz, 1H). Minor isomer (Z)¹H NMR (500 MHz, CD₃CN): δ_{H}=6.26 (d, J=9.3 Hz, 1H), 7.62-7.74 (m, 4H), 7.91-8.01 (m, 2H), 8.41 (d, J=9.8 Hz, 1H). ¹³C NMR (125 MHz, CD₃CN): δ_{c}=123.4, 127.5, 127.8, 128.4, 128.5, 135.9. IR (film, cm⁻¹): ν=2924.88 (C—H), 2122.21 (N—C). R_{f} value: 0.68 (20% ethyl acetate: 80% hexane)

### (E/Z)-2-Bromo-3-(2-isocyanovinyl)pyridine (33)

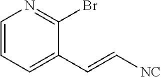

Following general procedure 1: Diisopropyl(isocyanomethyl)phosphonate (274 mg, 1.34 mmol), 2-bromo-3-pyridinecarboxaldehyde (100 mg, 0.53 mmol) and LiHMDS (1.62 mL, 1.62 mmol) were stirred in anhydrous THF (6 mL) for 18 hours. The title compound was purified by silica column chromatography (15% ethyl acetate/hexanes) to afford a dark yellow solid as a mixture of E- and Z-isomer in a 4:1 ratio (48 mg, 45%). Major isomer (E) ¹H NMR (500 MHz, CD₃CN): δ_{H}=6.55 (d, J=14.7 Hz, 1H), 7.15 (d, J=14.2 Hz, 1H), 7.88 (dd, J=2.0, 7.8 Hz, 1H), 8.35 (dd, J=2.0, 4.9 Hz, 1H). ¹³C NMR (125 MHz, CDCl₃): δ_{c}=123.3, 131.0, 132.8, 134.1, 135.0, 151.0. IR (film, cm⁻¹): ν=2923.79 (C—H), 2852.33 (C—H), 2111.64 (N—C). R_{f} value: 0.63 (20% ethyl acetate: 80% hexane)

### (E)-(2-Isocyanovinyl)cyclohexane (34)

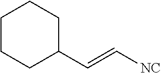

Following general procedure 1: Diisopropyl(isocyanomethyl)phosphonate (450 mg, 2.2 mmol), cyclohexylcarboxaldehyde (100 mg, 0.89 mmol) and LiHMDS (2.67 mL, 2.67 mmol) were stirred in anhydrous THF (7 mL) for 20 hours. The title compound was purified by silica column chromatography (50% ethyl acetate/hexanes) to afford a dark yellow solid as a single E-isomer (48 mg, 40%). ¹H NMR (500 MHz, CDCl₃): δ_{H}=1.05-1.30 (m, 5H), 1.65-1.80 (m, 4H), 1.99-2.09 (m, 1H), 5.60-5.65 (m, 1H), 6.06-6.12 (m, 1H). ¹³C NMR (125 MHz, CDCl₃): δ_{c}=25.5, 25.7, 31.6, 38.4, 86.6, 110.8, 142.4, 144.2, 161.9. IR (film, cm⁻¹): ν=2925.99 (C—H), 2853.28 (C—H), 2123.43 (N—C). R_{f} value: 0.83 (50% ethyl acetate: 50% hexane)

### 4-Hydroxy-2-styrylbenzaldehyde

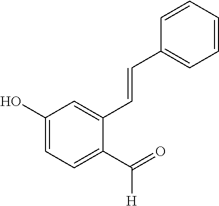 [SMILES: O=Cc1ccc(O)cc1/C=C/c1ccccc1]

Following general procedure 2: Triethylamine (0.41 mL, 2.98 mmol), styrene (0.3 mL, 2.98 mmol) 2-bromo-4-hydroxybenzaldehyde (400 mg, 1.98 mmol), Pd(OAc)₂ (45 mg, 0.19 mmol) and tri(o-tolyl)phosphine (120 mg, 0.39 mmol) were stirred overnight in DMF (10 mL). The title compound was obtained following purification by silica gel chromatography (15% ethyl acetate: 85% petroleum ether) to yield an orange/yellow solid (195 mg, 44%). ¹H NMR (500 MHz, CD₃OD): δ_{H}=6.85 (dd, J=2.9, 6.4 Hz, 1H), 7.10 (d, J=16.1 Hz, 1H), 7.15 (d, J=2.5 Hz, 1H), 7.28 (t, J=7.3 Hz, 1H), 7.35 (t, J=7.8 Hz, 2H), 7.56-7.57 (m, 1H), 7.75 (d, J=8.9 Hz, 1H), 8.12 (d, J=16.1 Hz, 1H), 10.07 (s, 1H). ¹³C NMR (125 MHz, CD₃OD): δ_{c}=112.8, 115.0, 124.7, 126.6, 128.3, 128.8, 133.1, 135.2, 191.2. IR (film, cm⁻¹): ν=3150.27 (O—H), 2926.00 (C—H), 1654.73 (C═O). R_{f} value: 0.22 (15% ethyl acetate: 85% petroleum ether). HRMS (ESI) calculated for C₁₅H₁₂O [M+H]⁺: Theoretical m/z=223.0757 Measured m/z=223.0759. Melting point: 197-199° C.

### 4-((E Z)-2-Isocyanovinyl)-3-((E)-styryl)phenol (36)

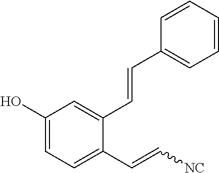

Following general procedure 1: Diisopropyl(isocyanomethyl)phosphonate (317 mg, 1.55 mmol), 4-hydroxy-2-styrylbenzaldehyde (120 mg, 0.51 mmol) and LiHMDS (2.07 mL, 2.07 mmol) were stirred in anhydrous THF (6 mL) for 18 hours. The title compound was purified by silica column chromatography (20% ethyl acetate/hexane) to afford a dark yellow solid as a mixture of E-Z-isomers in a ratio of 4:1(31 mg, 25%). Major isomer (E) ¹H NMR (500 MHz, CD₃OD): δ_{H}=6.35 (d, J=14.7 Hz, 1H), 6.72 (d, J=8.8 Hz, 1H), 6.97 (d, J=16.1 Hz, 1H), 7.02 (s, 1H), 7.27 (m, 2H), 7.30-7.40 (m, 4H), 7.55 (t, J=7.8 Hz, 1H), 7.60 (d, J=7.8 Hz, 2H). Minor isomer (Z)¹H NMR (500 MHz, CD₃OD): δ_{H}=6.05 (d, J=9.3 Hz, 1H), 6.77 (d, J=8.3 Hz, 1H), 6.85 (d, J=7.4 Hz, 2H), 7.00 (s, 1H), 7.10-7.20 (m, 3H), 7.30-7.40 (m, 3H), 7.65 (d, J=8.8 Hz, 1H), 7.85 (d, J=8.8 Hz, 1H). ¹³C NMR (125 MHz, CD₃OD): δ_{c}=111.1, 113.5, 116.4, 125.8, 127.5, 128.7, 128.7, 129.4, 131.2, 133.2, 135.5, 138.1, 139.4, 160.1. IR (film, cm⁻¹): v=3293.28 (O—H), 2924.82 (C—H), 2120.25 (N—C). R_{f} value: 0.27 (20% ethyl acetate: 80% hexane). HRMS (ESI) calculated for C₁₇H₁₃NO [M+H]⁺: Theoretical m/z=246.0917 Measured m/z=246.0918

### 5-Cinnamyl-2 hydroxybenzaldehyde

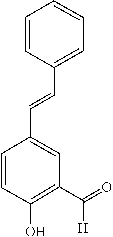 [SMILES: O=Cc1cc(/C=C/c2ccccc2)ccc1O]

Following general procedure 2: Triethylamine (0.63 mL, 4.47 mmol), styrene (51 mL, 4.47 mmol), 2-bromo-5-hydroxybenzaldehyde (600 mg, 2.98 mmol), Pd(OAc)₂(65 mg, 0.29 mmol) and tri(o-tolyl)phosphine (180 mg, 0.59 mmol) were stirred in DMF (10 mL). The title compound was obtained following silica gel chromatography (10% ethyl acetate: 90% petroleum ether) to yield a yellow solid (340 mg, 48%). ¹H NMR (500 MHz, CDCl₃): δ_{H}=7.01 (d, J=8.3 Hz, 1H), 7.05 (d, J=7.8 Hz, 1H), 7.28 (t, J=7.4 Hz, 1H), 7.38 (t, J=7.5 Hz, 3H), 7.51 (d, J=7.3 Hz, 2H), 7.66 (d, J=2.5 Hz, 1H), 7.72 (d, J=2.5 Hz, 1H), 9.95 (s, 1H), 11.02 (s, 1H). ¹³C NMR (125 MHz, CDCl₃): δ_{c}=118.1, 120.6, 126.3, 126.6, 127.7, 128.1, 128.7, 129.7, 131.5, 134.6, 136.9, 161.1, 196.5. IR (film, cm⁻¹): ν=3025.10 (O—H), 2853.34 (C—H), 1663.39 (C═O). R_{f} value: 0.42 (10% ethyl acetate: 90% petroleum ether). HRMS (ESI) calculated for C₁₅H₁₂O [M+H]⁺: Theoretical m/z=223.0756 Measured m/z=223.0759. Melting point: 195° C.

### 4-Cinnamyl-2-((E Z)-2-isocyanovinyl)phenol (37)

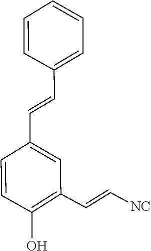

Following general procedure 1: Diisopropyl(isocyanomethyl)phosphonate (221 mg, 1.08 mmol), 5-cinnamyl-2 hydroxybenzaldehyde (100 mg, 0.43 mmol) and LiHMDS (1.30 mL, 1.30 mmol) were stirred in anhydrous THF (6 mL) for 20 hours. The title compound was purified by silica column chromatography (20% ethyl acetate/hexane) to afford a dark yellow solid as a single E-isomer (41 mg, 38%). ¹H NMR (500 MHz, CD₃CN): δ_{H}=6.75 (d, J=14.7 Hz, 1H), 6.93 (d, J=8.3 Hz, 1H), 7.10 (d, J=5.4 Hz, 2H), 7.30-7.40 (m, 2H), 7.36 (t, J=7.3 Hz, 2H), 7.60 (dd, J=2.5, 8.8 Hz, 1H), 7.58-7.61 (m, 3H). ¹³C NMR (125 MHz, CD₃CN): δ_{c}=117.8, 118.5, 118.9, 127.6, 126.9, 127.1, 128.3, 128.8, 128.9, 130.1, 130.2. IR (film, cm⁻¹): v=3245.54 (O—H), 2925.84 (C—H), 2853.53 (C—H), 2117.67 (N—C). R_{f} value: 0.64 (20% ethyl acetate: petroleum ether)

### (E)-2-Styrenebenzaldehyde

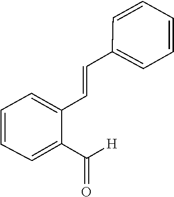 [SMILES: O=Cc1ccccc1/C=C/c1ccccc1]

Styrene (0.5 mL, 4.26 mmol) and 2-chlorobenzaldehyde (0.3 mL, 2.84 mmol) were added to a solution of Pd(OAc)₂ (64 mg, 0.28 mmol), Dave-phosphonate (67 mg, 0.17 mmol) and TBAE (1.71 g, 5.68 mmol) in dioxane (10 mL). The reaction mixture was allowed to stir at 80° C. for 48 h. Upon completion of the reaction (TLC confirmation), the resulting mixture was diluted with ethyl acetate, filtered through celite and concentrated under vacuo. The crude material was then purified on silica gel column chromatography (20% ethyl acetate: 80% petroleum ether) to yield the title compound as a yellow oil (340 mg, 51%). ¹H NMR (500 MHz, CDCl₃): δ_{H}=7.06 (d, J=16.1 Hz, 1H), 7.34 (t, J=7.3 Hz, 1H), 7.41 (t, J=7.3 Hz, 2H), 7.45 (t, J=7.3 Hz, 1H), 7.59-7.61 (m, 3H), 7.72 (d, J=7.8 Hz, 1H), 7.85 (d, J=7.8 Hz, 1H), 8.06 (d, J=16.1 Hz, 1H), 10.34 (s, 1H). ¹³C NMR (75 MHz, CDCl₃): δ_{c}=124.2, 126.5, 126.7, 127.1, 127.8, 128.3, 131.8, 132.3, 133.2, 133.4, 136.3, 139.3, 192.2. IR (film, cm⁻¹): ν=2923.50 (C—H), 2852.26 (C—H), 1692.38 (C═O). R_{f} value: 0.56 (20% ethyl acetate: 80% petroleum ether). HRMS (ESI) calculated for C₁₅H₁₂O [M+H]⁺: Theoretical m/z=209.0966 Measured m/z=209.0968

### 1-((E)-2-Isocyanovinyl)-2-((E)-styryl)benzene (38)

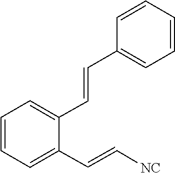

Following general procedure 1: Diisopropyl(isocyanomethyl)phosphonate (200 mg, 0.96 mmol), (E)-2-styrenebenzaldehyde (100 mg, 0.48 mmol) and LiHMDS (1.2 mL, 1.2 mmol) were stirred in anhydrous THF (6 mL) for 18 hours. The title compound was purified by silica column chromatography (60% ethyl acetate/hexanes) to afford a dark yellow solid as a single E-isomer (21 mg, 39%). ¹H NMR (500 MHz, CDCl₃): δ_{H}=6.20 (d, J=14.2 Hz, 1H), 6.99 (d, J=16.1 Hz, 1H), 7.28 (d, J=16.1 Hz, 1H), 7.29-7.43 (m, 7H), 7.55 (d, J=7.3 Hz, 2H), 7.61 (d, J=7.8 Hz, 1H). ¹³C NMR (75 MHz, CDCl₃): δ_{c}=124.9, 126.4, 126.5, 126.9, 127.6, 128.0, 128.6, 129.6, 132.7, 134.7, 136.5, 136.6. IR (film, cm⁻¹): ν=2923.48 (C—H), 2852.51 (C—H), 2122.62 (N—C). R_{f} value: 0.83 (60% ethyl acetate: 40% petroleum ether)

### E)-2-(2-(Pyridin-4-yl)vinyl)benzaldehyde

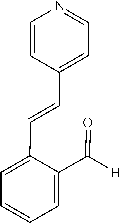 [SMILES: O=Cc1ccccc1/C=C/c1ccncc1]

Following procedure 2: 2-Bromobenzaldehyde (600 mg, 3.24 mmol), Pd(OAc)₂ (15 mg, 0.06 mmol), tri(o-tolyl)phosphine (40 mg, 0.13 mmol), 4-vinylpyridine (0.52 mL, 4.86 mmol) and triethylamine (1.30 mL, 9.72 mmol) were stirred in anhydrous DMF for 24 h. The crude material was purified using silica gel chromatography (50% ethyl acetate: 50% petroleum ether) to afford the title compound as a yellow oil (400 mg, 59%). ¹H NMR (500 MHz, CDCl₃): δ_{H}=6.95 (d, J=16.1 Hz, 1H), 7.40 (d, J=6.4 Hz, 1H), 7.40 (d, J=2.9 Hz, 1H), 7.51 (dt, J=1.5, 7.8 Hz, 1H), 7.61 (dt, J=1.0, 7.8 Hz, 1H), 7.72 (d, J=7.3 Hz, 1H), 7.83 (dd, J=1.5, 7.3 Hz, 1H), 8.29 (d, J=16.1 Hz, 1H), 8.59 (d, J=6.4 Hz, 1H), 8.59 (d, J=2.9 Hz, 1H), 10.24 (s, 1H). ¹³C NMR (125 MHz, CDCl₃): δ_{c}=121.1, 127.2, 128.5, 129.8, 130.5, 133.1, 133.5, 133.7, 138.2, 144.1, 150.2, 192.7. IR (film, cm⁻¹): ν=3033.36 (C—H), 2833.44 (C—H), 1689.97 (C═O). R_{f} value: 0.21 (50% ethyl acetate: 50% petroleum ether). HRMS (ESI) calculated for C₁₄H₁₁NO [M+H]⁺: Theoretical m/z=210.0925 Measured m/z=210.098

### 4-(E)-2-((E)-2-isocyanovinyl)styryl)pyridine (39)

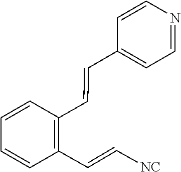

Following general procedure 1: Diisopropyl(isocyanomethyl)phosphonate (196 ng, 0.96 mmol), (E)-2-(2-(pyridin-4-yl)vinyl)benzaldehyde (100 mg, 0.48 mmol), and LiHMDS (1.20 mL, 1.20 mmol) were stirred in anhydrous THF (6 mL) for 18 hours. The title compound was purified by silica gel chromatography to afford a dark red oil as a single E-isomers (51 mg, 47%). ¹H NMR (500 MHz, CDCl₃): δ_{H}=6.90 (d, J=16.1 Hz, 1H), 7.31 (d, J=14.2 Hz, 1H), 7.33 (m, 1H), 7.37-7.44 (m, 4H), 7.48 (d, J=16.1 Hz, 1H), 7.62 (d, J=7.8 Hz, 1H), 8.63 (d, J=6.4 Hz, 1H), 8.63 (d, J=2.9 Hz, 1H). ¹³C NMR (125 MHz, CDCl₃): δ_{c}=120.8, 126.5, 127.0, 128.6, 129.3, 129.7, 130.0, 131.1, 135.2, 143.7, 150.1. IR (film, cm⁻¹): ν=3025.88 (C—H), 2123.15 (N—C). R_{f} value: 0.41 (65% ethyl acetate: 35% hexane). HRMS (ESI) calculated for C₁₆H₁₂N₂[M+H]⁺: Theoretical m/z=233.1078 Measured m/z=233.1088

### (E)-2-(2-(Pyrazin-2-yl)vinyl)benzaldehyde

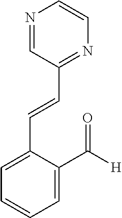 [SMILES: O=Cc1ccccc1/C=C/c1cnccn1]

Following general procedure 2: 2-Bromobenzaldehyde (1.2 g, 6.48 mmol), Pd(OAc)₂ (30 mg, 0.12 mmol), tri(o-tolyl)phosphine (80 mg, 0.26 mmol), 2-vinylpyrazine (1.0 mL, 9.72 mmol) and triethylamine (2.6 mL, 19.4 mmol) were stirred in anhydrous DMF for 24 h. The crude material was purified using silica gel chromatography (50% ethyl acetate: 50% petroleum ether) to afford the title compound as a yellow oil (860 mg, 63%). ¹H NMR (500 MHz, CDCl₃): δ_{H}=7.10 (d, J=16.1 Hz, 1H), 7.51 (t, J=7.8 Hz, 1H), 7.61 (t, J=7.8 Hz, 1H), 7.76 (d, J=7.3 Hz, 1H), 7.86 (d, J=7.3 Hz, 1H), 8.45 (d, J=4.4 Hz, 1H), 8.57 (d, J=1.5 Hz, 1H), 8.65 (d, J=16.1 Hz, 1H), 8.72 (d, J=3.4 Hz, 1H), 10.36 (s, 1H). ¹³C NMR (125 MHz, CDCl₃): δ_{c}=127.7, 129.0, 129.3, 131.7, 132.6, 133.7, 134.1, 138.6, 143.5, 144.1, 144.7, 151.0, 192.5. IR (film, cm⁻¹): ν=3063.44 (C—H), 2846.10 (C—H), 1686.49 (C═O). R_{f} value: 0.44 (50% ethyl acetate: 50% petroleum ether)

### 5-((E)-2-((E)-2-Isocyanovinyl)styryl)pyrimidine (40)

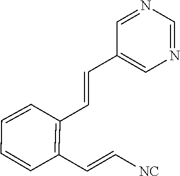

Following general procedure 1: Diisopropyl(isocyanomethyl)phosphonate (175 mg, 0.86 mmol), (E)-2-(2-(pyrazin-2-yl)vinyl)benzaldehyde (100 mg, 0.43 mmol) and LiHMDS (1.07 mL, 1.07 mmol) were stirred in anhydrous THF (6 mL) for 18 hours. The title compound was purified by silica gel chromatography to afford a dark red oil as a single E-isomers (45 mg, 41%). ¹H NMR (500 MHz, CDCl₃): δ_{H}=6.21 (d, J=14.2 Hz, 1H), 7.06 (d, J=15.7 Hz, 1H), 7.34-7.44 (m, 4H), 7.69 (d, J=7.8 Hz, 1H), 8.01 (d, J=15.7 Hz, 1H), 8.46 (d, J=2.5 Hz, 1H), 8.60 (m, 1H), 8.64 (d, J=1.5 Hz, 1H). ¹³C NMR (125 MHz, CDCl₃): δ_{c}=113.0, 126.6, 127.1, 127.5, 129.0, 129.9, 131.5, 131.7, 134.5, 135.4, 143.4, 144.0, 144.5, 150.5, 165.5. IR (film, cm⁻¹): ν=3064.81 (C—H), 2122.68 (N—C). R_{f} value: 0.58 (65% ethyl acetate: 35% hexane). HRMS (ESI) calculated for C₁₅H₁₁N₃[M+H]⁺: Theoretical m/z=234.1011 Measured m/z=234.1008

### 2-Bromo-4-nitrophenyl)methanol

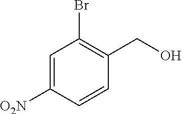 [SMILES: O=[N+]([O-])c1ccc(CO)c(Br)c1]

To a solution of 2-bromo-4-nitrobenzoic acid (500 mg, 2.00 mmol) in THE was added trimethylamine (0.3 mL, 2.00 mmol) and borane dimethylsulfide (6.0 mL, 6.00 mmol) at 0° C. The reaction was then heated at reflux for 3 h. Following this, the reaction mixture was allowed to cool to room temperature and slowly quenched with H₂O, acidified with concentrated HCl and further refluxed for 30 minutes. The reaction mixture was then extracted with DCM, dried with MgSO₄ and concentrated under vacuo to give the desired product as a yellow solid (464 mg, 100%). ¹H NMR (500 MHz, CD₃OD): δ_{H}=4.72 (s, 2H), 7.81 (d, J=8.3 Hz, 1H), 8.25 (dd, J=2.0, 8.3 Hz, 1H), 8.41 (d, J=2.4 Hz, 1H). ¹³C NMR (75 MHz, CD₃OD): δ_{c}=64.5, 121.7, 122.7, 127.6, 128.5, 147.3. IR (film, cm⁻¹): ν=3271.69 (O—H), 2916.75 (C—H), 2851.62 (C—H). R_{f} value: 0.45 (30% ethyl acetate: 70% petroleum ether). Melting point: 29° C.

### 2-Bromo-4-nitrobenzaldehyde

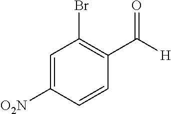 [SMILES: O=Cc1ccc([N+](=O)[O-])cc1Br]

Oxalyl chloride (0.2 mL, 2.58 mmol) was dissolved in DCM, cooled to −78° C. before DMSO (0.4 mL, 5.16 mmol) was added dropwise and the solution being stirred for 5 minutes. (2-bromo-4-nitrophenyl)methanol (400 mg, 1.72 mmol) was then added and allowed to stir for additional 1.5 h. Following this, triethylamine (1.2 mL, 8.60 mmol) was added and stirred for another 1.5 h, allowing the reaction to warm up to room temperature in the meantime. The reaction solution was then quenched with NaHCO₃, extracted with DCM with the organic phase dried with MgSO₄ and concentrated under vacuo to yield the desired compound as fine brown needles (350 mg, 87%). ¹H NMR (500 MHz, (CD₃)₂CO): δ_{H}=8.13 (d, J=8.8 Hz, 1H), 8.39-8.40 (m, 1H), 8.57 (d, J=2.0 Hz, 1H). ¹³C NMR (75 MHz, (CD₃)₂CO): δ_{c}=123.0, 125.7, 128.9, 130.9, 190.0. IR (film, cm⁻¹): ν=2955.86 (C—H), 2955.34 (C—H), 1518.35 (C═O). R_{f} value: 0.57 (30% ethyl acetate: 70% hexane). Melting point: 94° C.

### 2-(2-Bromo-4-nitrophenyl)-1,3-dioxane

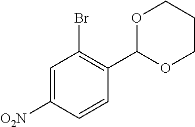 [SMILES: O=[N+]([O-])c1ccc(C2OCCCO2)c(Br)c1]

1,3 Propanediol (0.1 mL, 1.63 mmol) and p-TSA (20 mg, 0.11 mmol) was added to a solution of 2-bromo-4-nitrobenzaldehyde (250 mg, 1.09 mmol) in toluene. The reaction was left to stir overnight at 110° C. Once complete, the reaction was first cooled to room temperature before being quenched with H₂O. The reaction was then washed with H₂O and brine, extracted with toluene, dried with MgSO₄ and concentrated under vacuo to give the compound as a brown solid in quantitative yield. ¹H NMR (500 MHz, CDCl₃): δ_{H}=2.25 (m, 2H), 4.05 (m, 2H), 4.30 (m, 2H) 5.77 (s, 1H), 7.89 (d, J=8.8 Hz, 1H), 8.20 (dd, J=2.0, 8.3 Hz, 1H), 8.43 (d, J=8.4 Hz, 1H). ¹³C NMR (75 MHz, CD₃OD): δ_{c}=25.3, 68.6, 100.7, 123.3, 123.5, 128.4, 130.3, 145.2. IR (film, cm⁻¹): ν=2924.84 (C—H), 2880.65 (C—H). R_{f} value: 0.45 (25% ethyl acetate: 75% petroleum ether). Melting point: 120° C.

### (E)-2-(4-Nitro-2-styrylphenyl)-1,3-dioxane

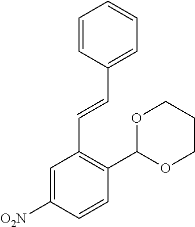 [SMILES: O=[N+]([O-])c1ccc(C2OCCCO2)c(/C=C/c2ccccc2)c1]

Following general procedure 2: 2-(2-bromo-4-nitrophenyl)-1,3-dioxane (200 mg, 0.69 mmol), Pd(OAc)₂ (16 mg, 0.07 mmol), tri(o-tolyl)phosphine (42 mg, 0.14 mmol), triethylamine (0.2 mL, 1.04 mmol) and styrene (0.1 mL, 1.04 mmol) were stirred in anhydrous DMF for 18 h. The title compound was purified using silica column chromatography (10% ethyl acetate: 90% petroleum ether) to afford a light yellow solid (130 mg, 61%). ¹H NMR (500 MHz, CDCl₃): δ_{H}=1.49-1.51 (m, 1H), 2.29-2.32 (m, 1H), 4.05-4.06 (m, 2H), 4.31-4.33 (m, 2H) 5.75 (s, 1H), 7.15 (d, J=16.1 Hz, 1H), 7.35 (t, J=7.3 Hz, 1H), 7.42 (t, J=7.3 Hz, 2H), 7.48 (d, J=16.1 Hz, 1H), 7.56 (d, J=7.8 Hz, 2H), 7.84 (d, J=8.8 Hz, 1H), 8.12 (dd, J=2.5, 8.8 Hz, 1H), 8.47 (d, J=2.5 Hz, 1H). ¹³C NMR (75 MHz, CDCl₃): δ_{c}=25.1, 67.8, 99.3, 121.5, 122.0, 123.9, 127.2, 128.1, 128.8, 129.1. IR (film, cm⁻¹): ν=2985.85 (C—H), 2846.84 (C—H), 1522.11 (C═O). R_{f} value: 0.40 (20% ethyl acetate: 80% petroleum ether). HRMS (ESI) calculated for C₁₈H₁₇NO₄ [M+H]⁺: Theoretical m/z=312.1235 Measured m/z=312.1203. Melting point: 160° C.

### (E)-4-Amino-2-styrylbenzaldehyde

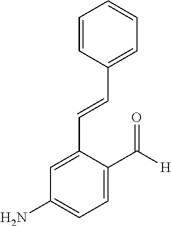 [SMILES: Nc1ccc(C=O)c(/C=C/c2ccccc2)c1]

Following general procedure 4: (E)-2-(4-Nitro-2-styrylphenyl)-1,3-dioxane (600 mg, 1.95 mmol) and iron powder (460 mg, 8.00 mmol) were stirred in a 5:1 ethanol/H₂O mixture before 1 mL saturated ammonium chloride was added. Following the completion of the reaction, the title compound was obtained as a yellow oil which was subsequently taken forward without further purification (400 mg, 93%). ¹H NMR (500 MHz, CDCl₃): δ_{H}=6.65 (dd, J=2.3, 8.3 Hz, 1H), 6.89 (d, J=2.3 Hz, 1H), 7.00 (d, J=16.2 Hz, 1H), 7.29-7.41 (m, 3H), 7.54-7.58 (m, 2H), 7.67 (d, J=8.3 Hz, 1H), 8.06 (d, J=16.2 Hz, 1H), 10.06 (s, 1H). IR (film, cm⁻¹): ν=3219.12 (N—H), 2998.84 (C—H), 2945.12 (C—H), 1677.95 (C═O). R_{f} value: 0.46 (35% ethyl acetate: 65% petroleum ether). HRMS (ESI) calculated for C₁₅H₁₃NO [M+H]⁺: Theoretical m/z=246.0894 Measured m/z=2.0873

### (E)-N-(4-Formyl-3-styrylphenyl)acetamide

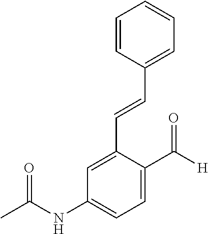 [SMILES: CC(=O)Nc1ccc(C=O)c(/C=C/c2ccccc2)c1]

Following general procedure 3: To a solution of (E)-2-(4-amino-2-styrylphenyl)-1,3-dioxane (400 mg, 2.22 mmol) in DCM was added acetic anhydride (0.3 mL, 2.60 mmol) and stirred overnight. The desired compound was isolated as a yellow oil in quantitative yield. ¹H NMR (500 MHz, CDCl₃): δ_{H}=2.23 (s, 3H), 7.04 (d, J=16.2 Hz, 1H), 7.28-7.32 (m, 1H), 7.37 (app t, J=7.8 Hz, 2H), 7.53 (d, J=7.8 Hz, 2H), 7.58 (d, J=8.3 Hz, 1H), 7.79 (d, J=8.3 Hz, 1H), 7.95 (s, 1H), 8.03 (d, J=16.1 Hz, 1H), 10.21 (s, 1H). ¹³C NMR (125 MHz, CDCl₃): δ_{c}=24.8, 116.9, 118.0, 124.4, 126.1, 127.0, 128.4, 128.6, 128.8, 129.0, 133.0, 134.0, 134.2, 136.7, 141.5, 142.8, 191.3. IR (film, cm⁻¹): ν=2992.21 (C—H), 29115.14 (C—H), 1687.93 (C═O). R_{f} value: 0.21 (35% ethyl acetate: 65% petroleum ether). HRMS (ESI) calculated for C₁₇H₁₅NO₂ [M+H]⁺: Theoretical m/z=266.1176 Measured m/z=266.1181

### N-(4-((E)-2-Isocyanovinyl)-3-((E)-styryl)phenyl)acetamide (41 and 42)

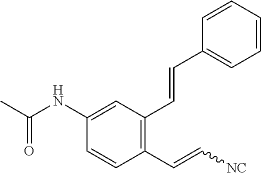

Following general procedure 1: Diisopropyl(isocyanomethyl)phosphonate (150 mg, 0.69 mmol), (E)-2-(4-nitro-2-styrylphenyl)-1,3-dioxane (120 mg, 0.36 mmol) and LiHMDS (0.9 mL, 0.9 mmol) were stirred in anhydrous THF (6 mL) for 18 hours. The title compound was purified by first silica gel chromatography and later purified using semi-preparative HPLC column chromatography (C18 reverse phase—90% acetonitrile/10% water) to afford a dark yellow solid as a mixture of isomers in a 6:1 ratio (63 mg, 49%). Major isomer (E) ¹H NMR (500 MHz, CDCl₃): δ_{H}=2.21 (s, 3H), 6.15 (d, J=14.2 Hz, 1H), 6.96 (d, J=15.7 Hz, 1H), 7.23 (d, J=16.1 Hz, 2H), 7.28-7.33 (m, 3H), 7.40 (t, J=7.3 Hz, 2H), 7.43 (d, J=8.3 Hz, 1H), 7.53 (d, J=7.3 Hz, 1H), 7.79 (s, 1H). Minor isomer (Z)¹H NMR (500 MHz, CDCl₃): δ_{H}=2.22 (s, 3H), 5.93 (d, J=9.3 Hz, 1H), 6.72 (d, J=9.3 Hz, 1H), 7.01 (d, J=16.1 Hz, 1H), 7.15 (d, J=16.1 Hz, 1H), 7.30-7.40 (m, 5H), 7.50 (t, J=7.3 Hz, 2H), 7.79 (d, J=8.3 Hz, 1H), 7.97 (s, 1H). ¹³C NMR (125 MHz, CDCl₃): δ_{c}=24.8, 110.0, 117.5, 119.0, 124.7, 126.8, 127.4, 128.4, 128.8, 133.4, 134.2, 136.7, 137.7, 139.3. IR (film, cm⁻¹): ν=3298.05 (N—H), 2908.28 (C—H), 2818.73 (C—H), 2114.81 (N—C), 1670.67 (C═O). R_{f} value: E=0.41, Z=0.50 (50% ethyl acetate: 50% petroleum ether). HRMS (ESI) calculated for C₁₉H₁₆N₂O [M+H]⁺: Theoretical m/z=287.1184 Measured m/z=287.1182

### (E)-N-(6-(2-Isocyanovinyl)-(1,1-biphenyl)-3-yl)acetamide (43)

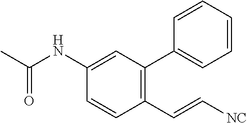

Following general procedure 1: Diisopropyl(isocyanomethyl)phosphonate (171 mg, 0.83 mmol), N-(6-formyl-(1,1-biphenyl)-3-yl)acetamide (100 mg, 0.42 mmol) and LiHMDS (1.05 mL, 1.05 mmol) were stirred in anhydrous THF (6 mL) for 20 hours. The title compound was purified by silica gel chromatography to afford a red/brown solid as a mixture of isomers in a 1:1 ratio (51 mg, 47%). Major isomer (E) ¹H NMR (500 MHz, CDCl₃): δ_{H}=2.19 (s, 3H), 6.15 (d, J=14.2 Hz, 1H), 6.98 (d, J=16.1 Hz, 1H), 7.26 (d, J=16.1 Hz, 1H), 7.27-7.28 (m, 1H), 7.45 (m, 4H), 7.50 (br s, 1H), 7.60 (d, J=7.8 Hz, 1H). Minor isomer (Z)¹H NMR (500 MHz, CDCl₃): δ_{H}=2.20 (s, 3H), 5.75 (d, J=9.3 Hz, 1H), 7.29 (d, J=8.3 Hz, 1H), 7.41-7.46 (m, 4H), 7.49 (d, J=8.3 Hz, 1H), 8.03 (d, J=8.3 Hz, 1H). ¹³C NMR (125 MHz, CDCl₃): δ_{c}=25.0, 118.5, 119.0, 121.1, 121.4, 126.8, 128.1, 128.5, 128.7, 129.7, 129.9, 131.3, 135.4, 139.2, 143.1, 168.5. IR (film, cm⁻¹): ν=3301.47 (N—H), 3083.77 (C—H), 2926.82 (C—H), 2110.25 (N—C). R_{f} value: E=0.67, Z=0.75 (70% ethyl acetate: 30% petroleum ether). HRMS (ESI) calculated for C₁₇H₁₄N₂O [M−H]⁺: Theoretical m/z=261.1027 Measured m/z=261.1019

### (E)-4-(2-(1,3-Dioxan-2-yl)-5-nitrostyryl)pyridine

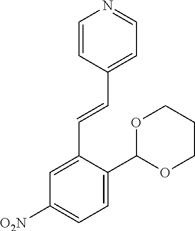 [SMILES: O=[N+]([O-])c1ccc(C2OCCCO2)c(/C=C/c2ccncc2)c1]

Following general procedure 2: 2-(2-bromo-4-nitrophenyl)-1,3-dioxane (400 mg, 1.42 mmol), Pd(OAc)₂ (16 mg, 0.12 mmol), tri(o-tolyl)phosphine (51 mg, 0.17 mmol), 4-vinylpyridine (0.16 mL, 1.54 mmol) and sodium acetate (230 mg, 2.84 mmol) were stirred in anhydrous DMF at 120° C. for 24 h. The crude material was purified using silica gel chromatography (80% ethyl acetate: 20% petroleum ether) to afford the title compound as a yellow solid (420 mg, 95%). ¹H NMR (500 MHz, CDCl₃): δ_{H}=1.59-1.61 (m, 1H), 2.30-2.32 (m, 1H), 4.11-4.13 (m, 2H), 4.29-4.31 (m, 2H) 5.75 (s, 1H), 7.15 (d, J=16.2 Hz, 1H), 7.40 (d, J=6.0 Hz, 2H), 7.76 (d, J=16.2 Hz, 1H), 7.42 (d, J=8.7 Hz, 1H), 8.17 (dd, J=2.3, 8.7 Hz, 1H), 8.49 (d, J=2.3 Hz, 1H), 8.65 (d, J=6.0 Hz, 1H), 8.65 (d, J=3.4 Hz, 1H). ¹³C NMR (125 MHz, CDCl₃): δ_{c}=25.9, 67.9, 99.6, 121.4, 121.5, 123.0, 128.5, 128.6, 130.9, 150.6. IR (film, cm⁻¹): ν=2968.65 (C—H), 2855.14 (C—H), 1593.91 (C═O). R_{f} value: 0.27 (80% ethyl acetate: 20% petroleum ether). HRMS (ESI) calculated for C₁₇H₁₆N₂O₄ [M−H]⁺: Theoretical m/z=283.2614 Measured m/z=283.2676. Melting point: 159° C.

### (E)-4-Amino-2-(2-(pyridin-4-yl)vinyl)benzaldehyde

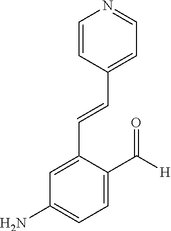 [SMILES: Nc1ccc(C=O)c(/C=C/c2ccncc2)c1]

Following procedure 4: (E)-4-(2-(1,3-dioxan-2-yl)-5-nitrostyryl)pyridine (400 mg, 1.28 mmol) and iron powder (300 mg, 5.12 mmol) was stirred in an ethanol/H₂O mixture for 3 h to give the title compound as a yellow oil which was subsequently taken forward to the next reaction without purification. ¹H NMR (500 MHz, CDCl₃): δ_{H}=6.70 (dd, J=2.3, 8.3 Hz, 1H), 6.90 (d, J=16.2 Hz, 1H), 6.90 (d, J=2.3 Hz, 1H), 7.42 (d, J=6.0 Hz, 1H), 7.42 (d, J=3.0 Hz, 1H), 7.65 (d, J=8.7 Hz, 1H), 8.33 (d, J=16.2 Hz, 1H), 8.60 (d, J=6.0 Hz, 1H), 8.60 (d, J=3.0 Hz, 1H), 9.99 (s, 1H). ¹³C NMR (125 MHz, CDCl₃): δ_{c}=112.0, 113.8, 121.0, 121.1, 121.2, 129.9, 130.7, 136.5, 140.7, 144.4, 150.1, 150.3, 151.4, 190.7. IR (film, cm⁻¹): ν=3353.83 (N—H), 3206.08 (N—H), 2924.62 (C—H), 2854.37 (C—H), 1593.69 (C═O). R_{f} value: 0.30 (100% ethyl acetate). HRMS (ESI) calculated for C₁₄H₁₂N₂O [M+H]⁺: Theoretical m/z=225.1027 Measured m/z=225.1021

### (E)-N-(4-Formyl-3-(2-(pyridin-4-yl)phenyl)acetamide

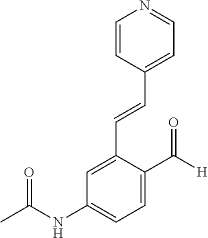 [SMILES: CC(=O)Nc1ccc(C=O)c(/C=C/c2ccncc2)c1]

Following general procedure 3: (E)-4-amino-2-(2-(pyridin-4-yl)vinyl)benzaldehyde (100 mg, 0.49 mmol) and acetic anhydride (0.1 mL, 0.60 mmol) were stirred in anhydrous DCM overnight to give the title compound as a yellow oil (quantitative). ¹H NMR (500 MHz, CD₃OD): δ_{H}=2.20 (s, 3H), 7.12 (d, J=16.2 Hz, 1H), 7.61 (d, J=5.9 Hz, 2H), 7.72 (dd, J=2.0, 8.3 Hz, 1H), 7.87 (d, J=8.3 Hz, 1H), 8.14 (d, J=2.0 Hz, 1H), 8.45 (d, J=16.1 Hz, 1H), 8.54 (d, J=5.9 Hz, 2H), 10.16 (s, 1H). ¹³C NMR (125 MHz, CD₃OD): δ_{c}=31.1, 114.5, 116.5, 126.5, 128.4, 129.6, 130.3, 134.7, 135.4, 139.0, 144.4, 164.6, 193.5. IR (film, cm⁻¹): ν=3068.97 (N—H), 2922.87 (C—H), 2852.51 (C—H), 1679.72 (C═O). R_{f} value: 0.10 (100% ethyl acetate). HRMS (ESI) calculated for C₁₆H₁₄N₂O₂ [M+H]⁺: Theoretical m/z=267.1133 Measured m/z=267.1120

### N-(4-((E)-2-Isocyanovinyl)-3-((E)-2-(pyridin-4-yl)vinyl)phenyl)acetamide (44)

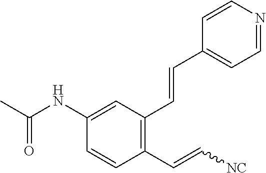

Following general procedure 1: Diisopropyl(isocyanomethyl)phosphonate (100 mg, 0.48 mmol), (E)-N-(4-formyl-3-(2-(pyridin-4-yl)phenyl)acetamide (65 mg, 0.24 mmol) and LiHMDS (0.61 mL, 0.61 mmol) were stirred in anhydrous THF (6.5 mL) for 20 hours. The title compound was purified by silica gel chromatography (100% ethyl acetate to 10% methanol:DCM gradient) to afford a red solid as a mixture of isomers in a 7:2 E:Z ratio (39 mg, 56%). Major isomer (E) ¹H NMR (500 MHz, CDCl₃): δ_{H}=2.23 (s, 3H), 6.17 (d, J=14.2 Hz, 1H), 6.92 (d, J=16.1 Hz, 1H), 7.31-7.40 (m, 5H), 7.45 (d, J=16.3 Hz, 1H), 7.93 (s, 1H), 8.64 (d, J=6.4 Hz, 2H). Minor isomer (Z)¹H NMR (500 MHz, CDCl₃): δ_{H}=2.18 (s, 3H), 5.97 (d, J=9.3 Hz, 1H), 6.72 (d, J=8.8 Hz, 1H), 6.95 (d, J=16.1 Hz, 1H), 7.25 (s, 1H) 7.31-7.46 (m, 5H), 7.79 (d, J=8.8 Hz, 1H), 8.11 (s, 1H), 8.61 (d, J=5.9 Hz, 1H). ¹³C NMR (125 MHz, CDCl₃): δ_{c}=24.7, 117.5, 119.5, 120.8, 127.2, 128.9, 130.5, 133.4, 133.6, 139.2, 143.6, 150.0, 150.1. IR (film, cm⁻¹): ν=2926.03 (C—H), 2855.11 (C—H), 2114.64 (N—C). R_{f} value: 0.2 (100% ethyl acetate). HRMS (ESI) calculated for C₁₈H₁₅N₃O [M+H]⁺: Theoretical m/z=290.1293 Measured m/z=290.1272

TABLE 3
The zone of inhibition (mm) obtained from screening the synthesised
compounds against strains MRSA 252, MSSA 476, S. aureus 15981, P.
aeruginosa PA01 and E. coli DH5α. Compounds 3-4, 19-
21, 27 and 32-35 were not active against any of the strains assayed
and were therefore emitted from the table.

| | Bacterial Strains (Zone of inhibition (mm)) | | | | |
|---|---|---|---|---|---|
| | | | Staphylo- | | Esche- |
| | | | coccus | Pseudomonas | richia |
| Compound | MRSA | MSSA | aureus | aeruginosa | coli |
| Number | 252 | 476 | 15981 | PA01 | DH5α |
| 2 | 22 | 20 | 22 | / | 20 |
| 5 | 26 | 30 | 16 | 26 | 18 |
| 10 | 20 | 16 | 18 | / | / |
| 11 | 18 | 20 | 14 | 12 | / |
| 12 | 16 | 14 | 16 | / | / |
| 13 | 22 | 22 | 18 | / | / |
| 14 | 24 | 24 | 28 | / | 26 |
| 15 | 20 | 22 | 24 | / | / |
| 16 | 22 | 22 | 24 | 18 | / |
| 17 | 18 | 18 | 16 | / | / |
| 18 | 14 | 26 | 20 | / | / |
| 22 | 20 | 16 | 18 | / | 20 |
| 23 | 18 | 20 | 14 | 14 | 14 |
| 24 | 16 | 22 | 18 | / | 14 |
| 25 | 18 | 16 | 16 | / | / |
| 26 | 20 | 20 | 22 | / | 20 |
| 28 | 26 | 22 | 22 | 20 | / |
| 29 | 22 | 16 | 16 | 12 | / |
| 30 | 14 | 12 | 12 | / | / |
| 31 | / | 12 | 12 | / | / |
| 36 | 28 | 30 | 30 | / | / |
| 37 | 22 | 24 | 22 | / | / |
| 38 | 30 | 30 | 28 | 26 | 28 |
| 39 | 28 | 32 | 26 | 20 | 12 |
| 40 | 24 | 26 | 28 | 12 | / |
| 41 | 32 | 34 | 34 | 16 | 20 |
| 42 | 26 | 30 | 28 | / | / |
| 43 | 22 | 18 | 18 | / | / |
| 44 | 28 | 28 | 30 | 26 | / |

TABLE 4
% Survival after 5 days of Galleria mellonella (n =
10) after injection of 500 μg/mL of antibiotic

| | % survival after 5 days |
|---|---|
| Compound number | Concentration (500 μg/mL) |
| Positive Control (PBS) | 80 |
| Negative Control (NaN₃) | 0 |
| 2 | 90 |
| 5 | 10 |
| 10 | 90 |
| 11 | 80 |
| 12 | 70 |
| 13 | 100 |
| 14 | 70 |
| 15 | 80 |
| 16 | 90 |
| 17 | 60 |
| 18 | 100 |
| 22 | 70 |
| 23 | 80 |
| 24 | 50 |
| 25 | 90 |
| 26 | 100 |
| 27 | 90 |
| 29 | 60 |
| 30 | 80 |
| 31 | 90 |
| 36 | 80 |
| 38 | 100 |
| 39 | 80 |
| 40 | 60 |
| 41 | 80 |
| 42 | 80 |
| 43 | 80 |
| 44 | 90 |

TABLE 5
Zone of inhibition (mm) for compounds 2, 13, 14, 22, 36 and 41
against various Staphylococcus aureus strains
Compound number

| | | | | | |
|---|---|---|---|---|---|
| 2 | 13 | 14 | 22 | 36 | 41 |

| | |
|---|---|
| Strain | Zone of inhibition (mm) |

| | | | | | | |
|---|---|---|---|---|---|---|
| EOE 3 | 12 | 24 | 20 | 20 | 30 | 26 |
| EOE 23 | 16 | 22 | 22 | 22 | 32 | 26 |
| EOE 30 | 18 | 22 | 22 | 20 | 34 | 30 |
| EOE 35 | 14 | 22 | 18 | 18 | 32 | 28 |
| EOE 41 | 17 | 20 | 20 | 20 | 28 | 26 |
| EOE 42 | 16 | 20 | 18 | 22 | 26 | 30 |
| EOE 45 | 15 | 24 | 20 | 26 | 28 | 30 |
| EOE 52 | 14 | 22 | 25 | 22 | 28 | 28 |
| EOE 54 | 16 | 22 | 19 | 16 | 29 | 26 |
| EOE 57 | 18 | 25 | 20 | 18 | 28 | 30 |
| USFL 008 | 14 | 18 | 17 | 14 | 26 | 28 |
| USFL 009 | 14 | 22 | 16 | 14 | 28 | 27 |
| USFL 012 | 16 | 22 | 20 | 16 | 28 | 32 |
| USFL 016 | 14 | 20 | 18 | 18 | 32 | 29 |
| USFL 018 | 15 | 20 | 18 | 18 | 30 | 32 |
| USFL 020 | 14 | 20 | 16 | 16 | 30 | 28 |
| USFL 021 | 16 | 22 | 18 | 16 | 29 | 29 |
| USFL 028 | 15 | 22 | 18 | 15 | 28 | 29 |
| USFL 035 | 15 | 22 | 19 | 16 | 28 | 30 |
| USFL 054 | 14 | 22 | 18 | 19 | 28 | 30 |
| MRSA 252 | 16 | 22 | 24 | 20 | 28 | 32 |
| Sa_TPS 3026 | 14 | 20 | 16 | 20 | 29 | 28 |
| Sa_TPS 3072 | 16 | 21 | 18 | 22 | 28 | 26 |
| Sa_TPS 3092 | 20 | 20 | 24 | 24 | 29 | 28 |
| Sa_TPS 3097 | 15 | 20 | 18 | 24 | 34 | 28 |
| Sa_TPS 3103 | 16 | 20 | 26 | 22 | 32 | 28 |
| Sa_TPS 3104 | 15 | 22 | 22 | 20 | 32 | 28 |
| Sa_TPS 3106 | 15 | 16 | 20 | 26 | 28 | 36 |
| Sa_TPS 3118 | 18 | 22 | 18 | 18 | 28 | 32 |
| Sa_TPS 3133 | 14 | 20 | 18 | 18 | 28 | 28 |
| Sa_TPS 3134 | 14 | 18 | 18 | 18 | 26 | 26 |
| ASARM 61 | 15 | 20 | 22 | 18 | 29 | 28 |
| ASARM 70 | 14 | 20 | 20 | 18 | 28 | 32 |
| ASARM 71 | 14 | 20 | 22 | 20 | 26 | 32 |
| ASARM 72 | 12 | 16 | 20 | 22 | 26 | 30 |
| ASARM 73 | 16 | 20 | 20 | 20 | 29 | 27 |
| ASARM 75 | 15 | 20 | 20 | 22 | 32 | 26 |
| ASARM 76 | 14 | 20 | 22 | 26 | 34 | 27 |
| ASARM 77 | 14 | 20 | 22 | 24 | 34 | 25 |
| ASARM 79 | 13 | 20 | 24 | 22 | 29 | 29 |
| ASARM 80 | 13 | 18 | 25 | 26 | 30 | 30 |
| S197_1_1 | 25 | 26 | 25 | 22 | 29 | 32 |
| S197_1_8 | 16 | 20 | 22 | 24 | 28 | 30 |
| S197_1_9 | 16 | 20 | 22 | 26 | 28 | 32 |
| S197_1_10 | 15 | 20 | 22 | 22 | 28 | 31 |
| | | | | 18 | 22 | 30 |
| S295_1_1 | 20 | 22 | 22 | 20 | 30 | 29 |
| S295_1_2 | 14 | 20 | 14 | 20 | 30 | 25 |
| S295_1_3 | 18 | 20 | 20 | 20 | 32 | 27 |
| S364_5_11 | 26 | 20 | 24 | 22 | 30 | 27 |
| S364_5_12 | 22 | 25 | 24 | 20 | 28 | 28 |
| S139_7_7 | 14 | 24 | 20 | 16 | 24 | 22 |

TABLE 6
Concentration of compound deemed as toxic to HEK 293 and red blood
cells. Compound 2 was not screened for this as the compound did
not pass primary screening assays (i.e. the compound was deemed
not active enough). The data was provided by CO-ADD.

| | Concentration | Hemolytic |
|---|---|---|
| | toxic to HEK | activity |
| | 293 cells | (HC₁₀) |
| Compound | (μg/mL) | (μg/mL) |
| 13 | >32 | >32 |
| 14 | >32 | >32 |
| 22 | >32 | >32 |
| 36 | >32 | >32 |
| 38 | >32 | >32 |
| 41 | >32 | >32 |

Development of a new (E)-selective HWE reagent for the synthesis of vinyl isocyanides and aglycone (E)-4 Initial synthetic efforts focused on preparing phenol vinyl isocyanide (E)-4.¹¹ Deprotonation of diethyl isocyanomethylphosphonate 11⁸ was carried out using 2.2 eq. of LHMDS in THF at −78° C. for 15 minutes, to allow for competing deprotection of the free phenol group of 12a. This gave the desired phenol vinyl isocyanide as a 7:3 mixture of its (E)-4 (J_{(2,3)}=14.4 Hz) and (Z)-4 (J_{(2,3)}=8.8 Hz) isomers in 56% yield (Scheme 4).¹²

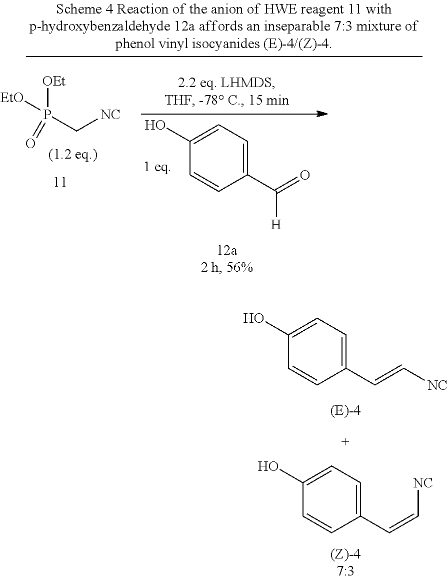

Unfortunately, this mixture of geometric isomers was difficult to separate by chromatography (silica, alumina), with significant mass losses occurring during these purification attempts.

We investigated whether HWE reaction of the anion of the more sterically demanding diisopropyl isocyanomethyl phosphonate reagent 13 and aldehyde 12a might result in improved (E)-selectivity.¹⁵ This new diisopropyl reagent 13 was prepared via modification of the 4-step literature protocol used previously to prepare Schollkopf's HWE reagent 11 (See SI for details).⁸

Reaction of the lithium anion of 1.1 eq. of the new HWE reagent 13 (generated using 2.2 eq. LHMDS) with 1 eq. of p-hydroxybenzaldehyde 12a in THE at −78° C. resulted in selective formation of (E)-4 in a 9:1 ratio over its corresponding (Z)-4 isomer. Furthermore, HWE reaction of 1.1 eq. of the lithium anion of HWE reagent 13 with p-TBSO-benzaldehyde 12b, afforded a further improved 95:5 mixture of the corresponding p-TBSO-phenol-vinyl isocyanide (E)-14 and p-TBSO-phenol-vinyl isocyanide (Z)-14. Base mediated O-silyl-deprotection of this mixture of geometric isomers via treatment with KOH in EtOH then gave the desired aglycone in an unchanged 95:5 ratio of (E)-4/(Z)-4 in 35% yield over two steps (Scheme 5). This aglycone proved to be highly reactive, readily polymerizing on standing at rt to afford black polymeric material. Aglycone (E)-4 could be stored as a dilute solution in acid free chloroform in the dark at −10° C. for several weeks without decomposition occurring. However, prolonged storage (months) of (E)-4 (95:5 dr) resulted in slow geometric isomerization to afford increasing amounts of its (Z)-4 isomer, ultimately resulting in a thermodynamic 7:3 mixture of (E)-4:(Z)-4 at equilibria.

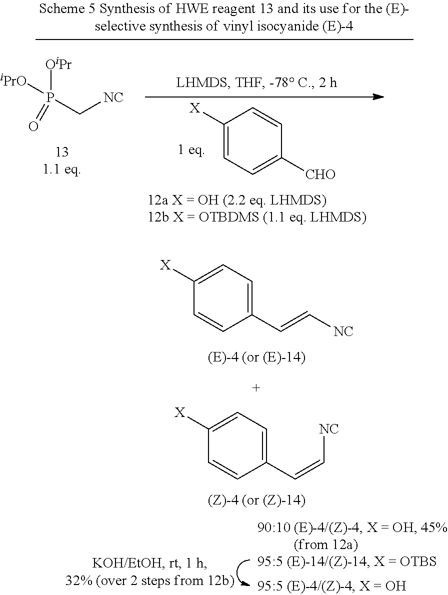

Since the anion of diisopropyl isocyanide phosphonate 13 (R=ⁱPr) had shown improved diastereoselectivities in HWE reactions with aldehydes 12a/12b, it was decided to explore its use for the preparation of a small series of synthetically useful (E)-vinyl isocyanides 15a-h (Table 7).¹⁶ The lithium enolate of HWE reagent 13 was reacted with a range of eight aldehydes in THE at −78° C. to afford their corresponding vinyl isocyanides 15a-h in 50-92% yield with >90:10 (E)-/(Z)-diastereomeric ratios in all cases (Table 7, column 3). Good (E)-selectivities were observed for the HWE reactions of electron rich aldehydes (Table 7, Entries 2-4), an electron deficient aromatic aldehyde (Table 7, Entry 5), a heteroaryl aldehyde (Table 7, Entry 6), an aliphatic aldehyde (Table 7, Entry 7) and a cyclic aldehyde (Table 7, Entry 8). The (E)-/(Z)-ratios obtained in the HWE reactions of 13 (R=ⁱPr) were all significantly greater than those for the corresponding HWE reactions of the lithium anion of the corresponding diethyl isocyanide phosphonate 11 (R=Et) (cf(E)-/(Z)-ratios reported in columns 3 and 4 of Table 7). For example, reaction of the lithium anion of HWE reagent 11 (R=Et) with p-nitrobenzaldehyde afforded vinyl isocyanide 15e in a poor 57:43 (E)-:(Z)-ratio, whilst the lithium anion of 14 (R=ⁱPr) gave 15e in a much improved 95:5 ratio in favor of its (E)-isomer (Table 7, entry 5).

TABLE
Comparison of the (E)-/(Z)-selectivities of the HWE reactions of the
lithium anions of 11 and 13 with a range of aldehydes.

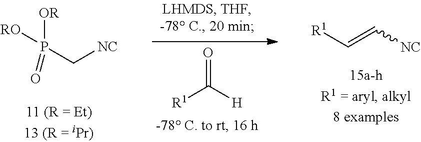

| | | (E)-/(Z)-Ratio^{a} | | % Yield |
|---|---|---|---|---|
| | | 11 | 13 | of 15 |
| Entry | Product | (R = Et) | (R = ⁱPr) | (from 13) |
| 1 | 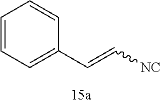 | 83:17 | 95:5 | 75 |
| 2 | 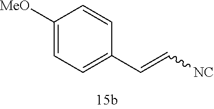 | 79:21 | 97:3 | 70 |
| 3 | 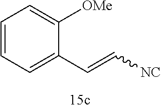 | 78:22 | 90:10 | 70 |
| 4 | 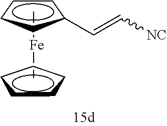 | 80:20 | 97:3 | 63 |
| 5 | 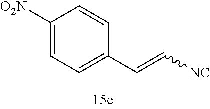 | 57:43 | 95:5 | 50^{b} |
| 6 | 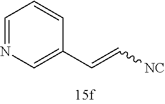 | 70:30 | 90:10 | 92 |
| 7 | 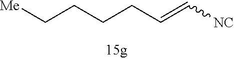 | 75:15 | >99:1 | 74 |
| 8 | 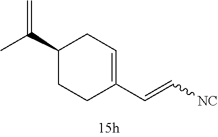 | 90:10 | >99:1 | 75 |

^{a}Ratios determined from the relative integrals of the well resolved α-protons of the (E)- /(Z)- isomers in the ¹H NMR spectra of the crude reaction products.
^{b}Low yield of 15e due to competing formation of its stable (anti)-β-hydroxy-phosphonate intermediate in 25% yield.

### Methods

### General Procedure for Carrying Out HWE Reactions

LHMDS (1.0 M in THF) (1.2 mL, 1.2 mmol) was added dropwise to a solution of diisopropyl (isocyanomethyl)phosphonate 13 (0.23 mL, 1.1 mmol) in dry THE (5 mL) at −78° C. and the resulting solution stirred for 20 minutes. An aldehyde (1.0 mmol) was then added dropwise at −78° C. and the stirred reaction mixture allowed to slowly warm to rt over 16 h. The reaction mixture was then quenched with phosphate buffer pH 7.0 (approx. 0.2 mL), extracted with EtOAc (10 mL) before being dried (MgSO₄) and the solvent removed in vacuo to afford a crude product that was purified by silica gel chromatography to give the desired (E)-vinyl isocyanide product.

### REFERENCES

1. J. O'Neil, “Review on Antimicrobial Resistance,” (2016).
2. M. J. Renwick, D. M. Brogan, E. Mossialos, A systematic review and critical assessment of incentive strategies for discovery and development of novel antibiotics. Journal of Antibiotics 69, 73-88 (2016).
3. L. Hall-Stoodley, J. W. Costerton, P. Stoodley, Bacterial biofilms: From the natural environment to infectious diseases. Nature Reviews Microbiology 2, 95-108 (2004).
4. D. Davies, Understanding biofilm resistance to antibacterial agents. Nature Reviews Drug Discovery 2, 114-122 (2003).
5. I. Olsen, Biofilm-specific antibiotic tolerance and resistance. European Journal of Clinical Microbiology & Infectious Diseases 34, 877-886 (2015).
6. A. S. Lynch, D. Abbanat, New antibiotic agents and approaches to treat biofilm-associated infections. Expert Opinion on Therapeutic Patents 20, 1373-1387 (2010).
7. M. Otto, Staphylococcal biofilms. Bacterial Biofilms 322, 207-228 (2008).
8. R. Falcon et al., High vancomycin MICs within the susceptible range in Staphylococcus aureus bacteraemia isolates are associated with increased cell wall thickness and reduced intracellular killing by human phagocytes. International Journal of Antimicrobial Agents 47, 343-350 (2016).
9. N. K. Qureshi, S. H. Yin, S. Boyle-Vavra, The Role of the Staphylococcal VraTSR Regulatory System on Vancomycin Resistance and vanA Operon Expression in Vancomycin-Resistant Staphylococcus aureus. Plos One 9, 7 (2014).
10. C. Veeresham, Natural products derived from plants as a source of drugs. J. Adv. Pharma. Technol. Res 3, 200-201 (2012).
11. T. F. Bumol, A. M. Watanabe, Genetic information, genomic technologies, and the future of drug discovery. Jama-Journal of the American Medical Association 285, 551-555 (2001).
12. A. L. Harvey, R. Edrada-Ebel, R. J. Quinn, The re-emergence of natural products for drug discovery in the genomics era. Nature Reviews Drug Discovery 14, 111-129 (2015).
13. G. M. Cragg, D. J. Newman, Natural products: A continuing source of novel drug leads. Biochimica Et Biophysica Acta-General Subjects 1830, 3670-3695 (2013).
14. D. J. Newman, G. M. Cragg, Natural Products as Sources of New Drugs from 1981 to 2014. Journal of Natural Products 79, 629-661 (2016).
15. P. Monciardini, M. Iorio, S. Maffioli, M. Sosio, S. Donadio, Discovering new bioactive molecules from microbial sources. Microbial Biotechnology 7, 209-220 (2014).
16. H. B. Bode, in Insect Biotechnology, A. Vilcinskas, Ed. (Springer-Verlag Berlin, Berlin, 2011), vol. 2, pp. 77-93.
17. J. M. Crawford, C. Portmann, X. Zhang, M. B. J. Roeffaers, J. Clardy, Small molecule perimeter defense in entomopathogenic bacteria. Proceedings of the National Academy of Sciences of the United States of America 109, 10821-10826 (2012).
18. V. S. Somvanshi et al., A Single Promoter Inversion Switches Photorhabdus Between Pathogenic and Mutualistic States. Science 337, 88-93 (2012).
19. D. C. Davis et al., Discovery and characterization of aryl isonitriles as a new class of compounds versus methicillin- and vancomycin-resistant Staphylococcus aureus. European Journal of Medicinal Chemistry 101, 384-390 (2015).
20. D. A. Smith, Pharmacokinetics and Metabolism in Drug Design. (Wiley-VCH, ed. 3, 2012), vol. 51.
21. N. Brown, Bioisosteres and Scaffold Hopping in Medicinal Chemistry. Molecular Informatics 33, 458-462 (2014).
22. I. Hoppe, U. Schollkopf, SYNTHESIS AND BIOLOGICAL-ACTIVITIES OF THE ANTIBIOTIC-B-371 AND ITS ANALOGS. Liebigs Annalen Der Chemie, 600-607 (1984).
23. L. L. Silver, Challenges of Antibacterial Discovery. Clinical Microbiology Reviews 24, 71-+(2011).
24. I. Ugi, U. Fetzer, U. Eholzer, H. Knupfer, Offerman. K, ISONITRILE SYNTHESES. Angewandte Chemie-International Edition 4, 472-& (1965).
25. A. P. Desbois, P. J. Coote, in Advances in Applied Microbiology, Vol 78, A. I. Laskin, S. Sariaslani, G. M. Gadd, Eds. (Elsevier Academic Press Inc, San Diego, 2012), vol. 78, pp. 25-53.
26. C. P. Silva et al., Bacterial infection of a model insect: Photorhabdus luminescens and Manduca sexta. Cellular Microbiology 4, 329-339 (2002).
27. B. R. Boles, A. R. Horswill, agr-mediated dispersal of Staphylococcus aureus biofilms. Plos Pathogens 4, 13 (2008).
28. C. f. O. A. D. Discovery, “Hit Confirmation of Antibiotics,” (2016).
29. H. Bilgin, A. Eren, S. Kara, Hemolytic Anemia and Heart Failure Caused by Anti-C and Anti-E Immunization. Jcpsp-Journal of the College of Physicians and Surgeons Pakistan 26, 539-540 (2016).
30. R. Ramozzi, N. Cheron, B. Braida, P. C. Hiberty, P. Fleurat-Lessard, A valence bond view of isocyanides' electronic structure. New Journal of Chemistry 36, 1137-1140 (2012).
31. L. L. Ling et al., A new antibiotic kills pathogens without detectable resistance (vol 517, pg 455, 2015). Nature 520, (2015).
32. G. D. Brown et al., Hidden Killers: Human Fungal Infections. Science Translational Medicine 4, 9 (2012).
33. T. Roemer, D. J. Krysan, Antifungal Drug Development: Challenges, Unmet Clinical Needs, and New Approaches. Cold Spring Harbor Perspectives in Medicine 4, 14 (2014).

All publications mentioned in the above specification are herein incorporated by reference. Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

1. A compound of formula (I) or formula (II): 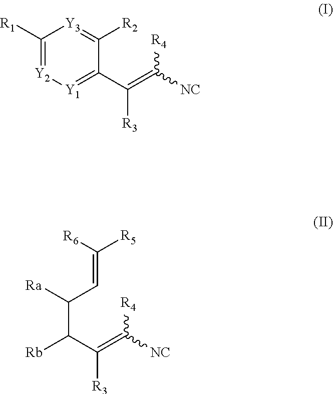
or a salt, solvate, diastereomer or tautomer thereof, wherein:
Y₁, Y₂ and Y₃ are independently selected from C—R₁ or N;
each R₁ is independently selected from H, C₁ to C₆ alkyl, OH, OR, NHCOR, NHSO₂R, CONHR, CONHSO₂R, substituted or unsubstituted aryl, substituted or unsubstituted heteroaryl or R₇; and each R is independently selected from H, or C₁ to C₆ alkyl;
R₂ is selected from substituted or unsubstituted aryl, substituted or unsubstituted heteroaryl, or R₇;
R₃ and R₄ are independently selected from H, or C₁ to C₆ alkyl;
R₇ is a group of formula: 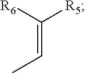
Rₐ and R_{b} are independently selected from H, or C₁ to C₆ alkyl; or Rₐ and R_{b} together with the atoms to which they are attached, form a substituted or unsubstituted 5 or 6 membered ring;
R₅ is selected from substituted or unsubstituted aryl or heteroaryl; and
R₆ is selected from H, or C₁ to C₆ alkyl.

2. A compound as claimed in claim 1, wherein the substituted or unsubstituted 5 or 6 membered ring comprises a substituted or unsubstituted cyclyl or heterocyclyl ring, suitably a C₅₋₂₀ cyclyl or C₅₋₁₀ heterocyclyl.

3. A compound as claimed in claim 1 or claim 2, wherein the substituted or unsubstituted 5 or 6 membered ring comprises a substituted or unsubstituted aryl or heteroaryl ring or a ring forming one ring of a fused ring structure, suitably wherein the substituted or unsubstituted 5 or 6 membered ring comprises a substituted or unsubstituted C₅₋₂₀ aryl or C₅₋₁₀ heteroaryl.

4. A compound as claimed in any one of the preceding claims, wherein the substituted or unsubstituted 5 or 6 membered ring is selected from pyrrolidine, pyrrole, pyridine, furan, thiophene, oxazole, isoxazole, isoxazine, oxadiazole (e.g. 1-oxa-2,3-diazolyl, 1-oxa-2,4-diazolyl, 1-oxa-2,5-diazolyl, 1-oxa-3,4-diazolyl), oxatriazole, thiazole, isothiazole, imidazole, pyrazole, pyridazine, pyrimidine, pyrazine, triazole (e.g. 1,2,4-triazole), triazine (e.g. 1,2,4-triazine), tetrazole, azaindole (e.g. 5-azaindole or 7-azaindole), azaindazole (e.g. 7-azaindolazole), azabenzimidazole (e.g. 5-azabenzimidazole), benzofuran, isobenzofuran, indole, quinoline, quinazoline, isoindole, indolizine, isoindoline, benzothiofuran, benzoxazole, benzisoxazole, benzothiazole, benzimidazole, indazole, benzodioxole, benzofurazan, benzothiadiazole, benzotriazole, purine (e.g., adenine, guanine), pyrrolo[1,2-a]pyrazine, pyrazolo[1,5-a]pyridine, 1H-pyrazolo[3,4-d]pyrimidine, pyrazolo[1,5-b]pyridazine, and pteridine.

5. A compound as claimed in any one of the preceding claims, wherein the compound is of formula (III): 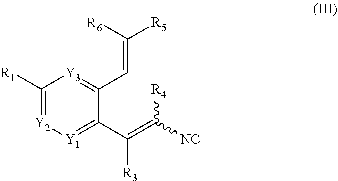
wherein R₁ is selected from H, C₁ to C₆ alkyl, OH, OR, NHCOR, NHSO₂R, CONHR, CONHSO₂R or substituted or unsubstituted aryl or substituted or unsubstituted heteroaryl;
Y₁, Y₂ and Y₃ are independently selected from C—R₁ or N; and
each R is independently selected from H, or C₁ to C₆ alkyl.

6. A compound as claimed in any one of the preceding claims, wherein the compound is of formula (IV): 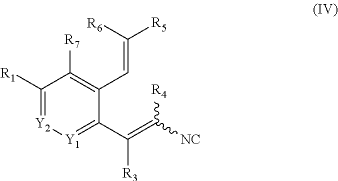
wherein R₆ and R₇ are independently selected from H, C₁ to C₆ alkyl, OH, or OR; or R₆ and R₇ together with the atoms to which they are attached form a substituted or unsubstituted 6 membered ring.

7. A compound as claimed in claim 6, wherein the compound is of formula (V): 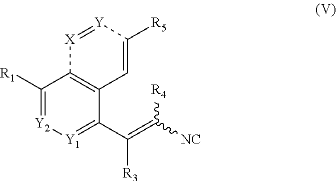
wherein the dotted lines to X and Y each independently indicate the optional presence of a bond; and X and Y are each independently selected from C(R)ₙ or N(R)ₘ; wherein n is 1 or 2 and m is 0 or 1 depending on the optional presence of a bond; with the proviso that at least one of the dotted lines to X and Y indicate the presence of a bond.

8. A compound as claimed in any one of claim 1 to 6, wherein the compound is of formula (V):

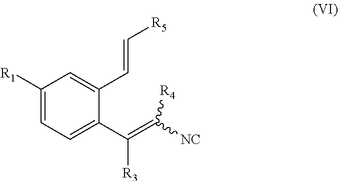

9. A compound as claimed in any one of the preceding claims, wherein R₅ is substituted or unsubstituted aryl, pyridyl, pyrazyl, pyridazyl or pyrimidyl.

10. A compound as claimed in any one of the preceding claims, wherein R₃ and R₄ are each H.

11. A compound as claimed in any one of the preceding claims, wherein each of Y₁, Y₂ and Y₃ are each C—R₁.

12. A compound as claimed in any one of the preceding claims, wherein at least one R₁ is selected from OH, OR, NHCOR, NHSO₂R, CONHR, CONHSO₂R, wherein each R is independently selected from H, or C₁ to C₆ alkyl.

13. A compound as claimed in any one of the preceding claims, wherein the compound is selected from compounds of formulae:

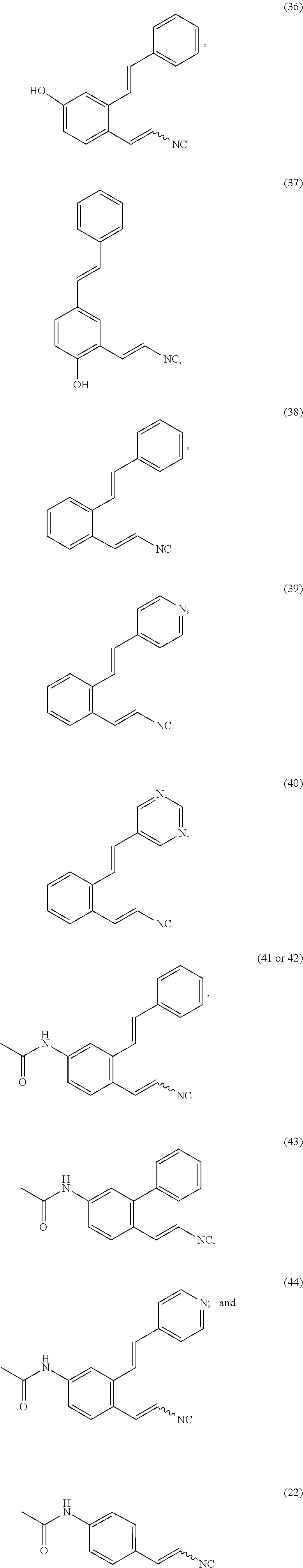

14. A method of producing a vinyl isocyanide compound of claim 1, the method comprising:
a) providing a phosphonate of formula (X): 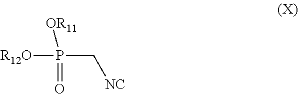
wherein R₁₁ and R₁₂ are independently selected from C₃ to C₅ alkyl, optionally independently selected from isopropyl, isobutyl, and t-butyl.
b) reacting the phosphonate with a carbonyl compound in the presence of base.

15. A method as claimed in claim 14, wherein the carbonyl compound is a compound of formula (XI) or (XII): 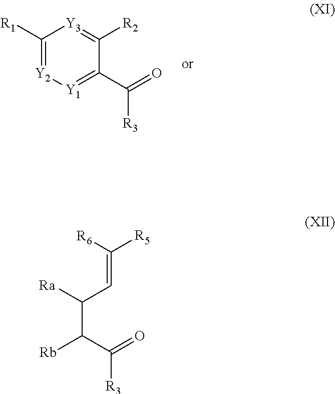
wherein, Y₁, Y₂ and Y₃ are independently selected from C—R₁ or N;
each R₁ is independently selected from H, C₁ to C₆ alkyl, OH, OR, NHCOR, NHSO₂R, CONHR, CONHSO₂R, substituted or unsubstituted aryl, substituted or unsubstituted heteroaryl or R₇; and each R is independently selected from H, or C₁ to C₆ alkyl;
R₂ is selected from substituted or unsubstituted aryl, substituted or unsubstituted heteroaryl, or R₇;
R₃ is selected from H, or C₁ to C₆ alkyl;
R₇ is a group of formula: 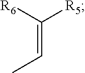
Rₐ and R_{b} are independently selected from H, or C₁ to C₆ alkyl; or Rₐ and R_{b} together with the atoms to which they are attached, form a substituted or unsubstituted 5 or 6 membered ring;
R₅ is selected from substituted or unsubstituted aryl or heteroaryl; and
R₆ is selected from H, or C₁ to C₆ alkyl.

16. A method as claimed in either claim 14 or claim 15, wherein the base comprises a non-nucleophilic base, optionally a Li base, optionally a base selected from lithium bis(trimethylsilyl)amide (LHMDS), lithium tetramethylpiperidide (LiTMP), and lithium diisopropylamide (LDA).

17. A method as claimed in any one of claims 14 to 16 wherein R₃ is H.

18. A method as claimed in any one of claims 14 to 17, wherein the phosphonate is reacted with a carbonyl in the presence of base and THE as solvent.

19. A reagent for use in the method of claim 14, the reagent comprising a compound of formula (X): 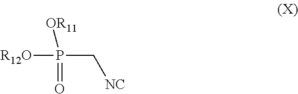
wherein R₁₁ and R₁₂ are independently selected from C₃ to C₅ alkyl, optionally independently selected from isopropyl, isobutyl, and t-butyl.

20. A compound of formula (I) or (II) and salts and solvates thereof according to any one of claims 1 to 13 for use as a medicament.

21. A compound of formula (I) or (II) and salts and solvates thereof according to any one of claims 1 to 13 for use in the treatment of an infectious disease.

22. A compound of formula (I) or (II) and salts and solvates thereof according to claim 21 for use in the treatment of a bacterial, fungal or protozoal disease.

23. A compound of formula (I) or (II) and salts and solvates thereof according to claim 22, wherein the bacterial disease is a disease caused by gram negative bacteria, or gram positive bacteria.

24. A pharmaceutical composition comprising a compound of formula (I) or (II) and salts and solvates thereof of any one of claims 1 to 13 and a pharmaceutically acceptable excipient, carrier or diluent.

25. The use of a compound of formula (I) or (II) and salts and solvates thereof according to any one of claims 1 to 13 in the manufacture of a medicament, optionally for treating an infectious disease.

26. A method of treatment of a subject, comprising administering to said subject a therapeutically effective amount of a compound of any one of claims 1 to 13 or a pharmaceutical composition of claim 17, optionally wherein the subject is suffering from an infectious disease.
